(19) United States
(12) Patent Application Publication
(10) Pub. No.: US 2024/0079144 A1
(43) Pub. Date: Mar. 7, 2024

(54) SYSTEMS AND METHODS OF PROMOTING EUBIOSIS IN PREGNANT OR BREASTFEEDING WOMEN

(71) Applicant: Brooklyn InnoSeq Inc., New York, NY (US)

(72) Inventor: Nini FAN, New York, NY (US)

(21) Appl. No.: 18/218,239

(22) Filed: Jul. 5, 2023

Related U.S. Application Data
(63) Continuation of application No. 17/984,602, filed on Nov. 10, 2022, now abandoned.
Application No. 17/984,602 is a continuation of application No. PCT/US21/31839, filed on May 11, 2021.
(60) Provisional application No. 63/022,887, filed on May 11, 2020.

(51) Int. Cl. G16H 50/30; C12Q 1/6869; G16H 20/60

(52) U.S. Cl. CPC G16H 50/30; C12Q 1/6869; G16H 20/60

(57) ABSTRACT

Described herein are methods and systems of promoting eubiosis or treating dysbiosis. In some embodiments, the subjects of the technology can include pregnant or breastfeeding women. In some embodiments, the subjects of the technology can include infants. Also described herein are sampling kits and methods of providing said kits to subjects of interest.

### CROSS-REFERENCE

This application is a continuation of U.S. application Ser. No. 17/984,602, filed Nov. 10, 2022, which is a continuation of International Application No. PCT/US21/31839, filed May 11, 2021, which claims the benefit of U.S. Provisional Application No. 63/022,887, filed May 11, 2020, each of which is incorporated by reference herein in its entirety.

### BACKGROUND

Responses to the same foods can differ significantly among individuals. For example, postprandial glycemic response (PPGR) of an individual can be driven by the glycemic index of a food, as well as the individual's phenotypic and molecular characteristics, including the gut microbiome. Nucleic acid sequencing can identify certain traits of an individual, including gut activity.

### SUMMARY

In some embodiments, the invention provides a method comprising analyzing a result of a gut microbiome sequencing assay of a biological sample of a subject to provide an outcome, and determining a health status of the subject based on the outcome, wherein the subject is a female within about a year of giving birth.

In some embodiments, the invention provides a method comprising analyzing a result of a microbiome sequencing assay of a biological sample of a subject to provide an outcome, and determining a recommended dietary regimen for the subject based on the outcome, wherein the subject is a female within about a year of giving birth.

In some embodiments, the invention provides a computer program product encoded on a non-transitory computer-readable medium comprising code that instructs performance of a method, the method comprising: a) providing a computational nutrition system, the computational nutrition system comprising: 1) a data receiving module; 2) a data processing module; 3) a health status module; and 4) a reporting module; b) receiving by the data receiving module data obtained from a gut microbiome assay of a subject, wherein the subject is a female subject who is within about a year of giving birth; c)
processing by the data processing module the data obtained from the gut microbiome assay of the subject to provide an assay result; d) analyzing by the health status module the assay result to provide an analysis of a health status of the subject, wherein the health status characterizes an effect of gut microbiome activity on reproductive processes within about a year of giving birth; and e) reporting to the subject by the reporting module the analysis of the health status of the subject.

In some embodiments, the invention provides a computer program product encoded on a non-transitory computer-readable medium comprising code that instructs performance of a method, the method comprising: a) providing a computational nutrition system, the computational nutrition system comprising: 1) a data receiving module; 2) a data processing module; 3) a nutrition recommendation module; and 4) a nutrition reporting module; b) receiving by the data receiving module data obtained from a microbiome assay of a subject, wherein the subject is a female subject who is within about a year of giving birth; c) processing by the data processing module the data obtained from the microbiome assay of the subject to provide an assay result; d) determining by the nutrition recommendation module a recommended dietary regimen for the subject based on the assay result; and e) reporting to the subject by the nutrition reporting module the recommended dietary regimen for the subject.

In some embodiments, the invention provides a kit comprising: a) a sheet of material configured to be applied to a toilet seat such that when a user sits on the toilet seat, the sheet of material forms a receptacle underneath an anus of the user; b) disposable gloves; c) a sample vial with a cap that forms a seal to the sample vial, wherein the cap comprises an elongated utensil component, wherein when the cap is sealed to the sample vial, the elongated utensil component extends into the sample vial; d) a specimen pouch suitable to contain the sample vial; and e) a shipping box suitable to contain the specimen pouch.

In some embodiments, the invention provides a method comprising: a) sending to a subject a kit, wherein the kit comprises: 1) a sheet of material configured to be applied to a toilet seat such that when a user sits on the toilet seat, the sheet of material forms a receptacle underneath an anus of the user; 2) disposable gloves; 3) a sample vial with a cap that forms a seal to the sample vial, wherein the cap comprises an elongated utensil component, wherein when the cap is sealed to the sample vial, the elongated utensil component extends into the sample vial; 4) a specimen pouch suitable to contain the sample vial; and 5) a shipping box suitable to contain the specimen pouch; and b) receiving from the subject the shipping box with the sample vial packed within the specimen pouch, with the specimen pouch packed within the shipping box, wherein the sample vial contains a biological sample of the subject when packed within the shipping box, wherein the subject is a female within about a year of giving birth.

In some embodiments, the invention provides a method comprising: a) sending by a subject a biological sample of the subject to a health service provider; and b) receiving by the subject from the health service provider a recommended dietary regimen for the subject based on an assay of the biological sample of the subject, wherein the subject is a female within about a year of giving birth.

In some embodiments, the invention provides a system comprising: a) a computer hardware that: 1) receives a result of a microbiome assay of a subject; and 2) determines based on the result of the microbiome assay a recommended dietary regimen for improving a health status of the subject, wherein the determining is based in part on the subject being a female within about a year of giving birth; and b) a nutrition distribution component that sends to the subject a supply of nutrition that corresponds to the recommended dietary regimen.

In some embodiments, the invention provides a computer program product encoded on a non-transitory computer-readable medium comprising code that instructs performance of a method, the method comprising: a) providing a computational nutrition system, the computational nutrition system comprising: 1) a data receiving module; 2) a data processing module; and 3) a nutrition recommendation module; b) receiving by the data receiving module data obtained from a microbiome assay of a subject, wherein the subject is a female within about a year of giving birth; c) processing by the data processing module the data obtained from the microbiome assay to provide an assay result; and d) determining by the nutrition recommendation module a recommended dietary regimen for the subject based on the assay result, wherein the determining is based in part on the subject being a female within about a year of giving birth.

In some embodiments, the invention provides a method comprising: a) receiving from a subject a report of nutritional intake of the subject; and b) providing to the subject a revised dietary regimen based on the report, wherein the subject is a female within about a year of giving birth who is undergoing a dietary regimen based on an analysis of a microbiome assay of the subject.

In some embodiments, the invention provides a method comprising: a) providing by a subject a report of nutritional intake of the subject to a health service provider; and b) receiving by the subject a revised dietary regimen from the health service provider based on the report of nutritional intake, wherein the subject is a female within about a year of giving birth who is undergoing a dietary regimen based on an analysis of a microbiome assay of the subject.

In some embodiments, the invention provides a computer program product encoded on a non-transitory computer-readable medium comprising code that instructs performance of a method, the method comprising: a) providing a computational nutrition system, the computational nutrition system comprising: 1) an update receipt module; 2) a revision module; and 3) a revision reporting module; b) receiving by the update receipt module an update from a subject, wherein the update describes nutritional intake by the subject; c) revising by the revision module the recommended dietary regimen for the subject based on the update from the subject to provide a revised dietary regimen, wherein the revised dietary regimen accounts for an effect of microbiome activity on a health status of the subject, wherein the health status characterizes an effect of gut microbiome activity on reproductive processes within about a year of giving birth; and d) reporting to the subject by the revision reporting module the revised dietary regimen.

In some embodiments, the invention provides a method for identifying a likelihood of a pregnancy-related condition in a subject, the method comprising: a) assaying microbial nucleic acids from a biological sample of the subject, thereby generating a microbiome profile of the subject; and b) identifying the likelihood of the pregnancy-related condition in the subject based on the microbiome profile of the subject.

In some embodiments, the invention provides a method for identifying a likelihood of a pregnancy-related condition in a subject, the method comprising: a) assaying microbial nucleic acids from a biological sample of the subject to detect a set of biomarkers; and b) computer processing the set of biomarkers with a trained algorithm to identify the likelihood of the pregnancy-related condition in the subject.

In some embodiments, the invention provides a system for detecting a pregnancy-related condition in a subject, the system comprising: a) a computer-readable medium comprising a machine learning model classifier operable to classify a likelihood of the pregnancy-related condition in the subject based on a microbiome profile of the subject; and b) a processor for executing instructions stored on the computer-readable medium.

In some embodiments, the invention provides a method of treating a dysbiosis in a subject in need thereof, the method comprising: a) obtaining a microbiome composition of the subject; b) determining a personalized nutritional therapy to the subject based on the microbiome composition of the subject; and c) supplying to the subject a supply of nutrition based on the personalized nutritional therapy, wherein the personalized nutritional therapy treats the dysbiosis in the subject.

In some embodiments, the invention provides a method of treating or reducing a likelihood of a pregnancy-related condition in a subject in need thereof, the method comprising: a) receiving by a subject from a telecommunications device a personalized nutritional therapy, wherein the personalized nutritional therapy is based on a microbiome profile of the subject; and b) adhering by the subject to the personalized nutritional therapy over a time period, wherein the pregnancy-related condition is improved after the subject adheres to the personalized nutritional therapy over the time period, wherein the time period is at least a week.

In some embodiments, the invention provides methods and systems of promoting eubiosis or treating dysbiosis in a subject in need thereof. In some embodiments, the subject is a pregnant or breastfeeding woman. In some embodiments, the subject is an infant. Also described herein are sampling kits and methods of providing said kits to subjects of interest.

In some embodiments, the invention provides a method for diagnosing and treating a dysbiosis in a pregnant or breastfeeding female subject, the method comprising: (a) receiving microbial sequence data comprising a microbiome feature dataset from an aggregate set of samples from a population of pregnant or breastfeeding female individuals; (b) receiving a sample from the subject; (c) determining microorganism nucleic acid sequences for the subject sample, comprising: (i) fragmenting nucleic acid material of the sample, and (ii) amplifying the fragmented nucleic acid material with a set of primers; (d) determining an alignment of the microorganism nucleic acid sequences to reference nucleic acid sequences in a database; (e) generating a microbiome feature dataset for the subject based upon the alignments; (f) generating a characterization of the subject's dysbiosis based upon: (i) a supplementary dataset informative of at least one characteristic associated with the population of individuals and the subject, and (ii) the microbiome feature datasets of the population of individuals and the subject; (g) based upon the characterization, generating a dietary regimen for correcting the dysbiosis; and (h) at an output device associated with the subject, providing the dietary regimen to the subject with the dysbiosis based upon the characterization.

In some embodiments, the invention provides a system for promoting maternal and/or fetal wellbeing, comprising: (a) a sampling kit including a sample container having a pre-process reagent component and configured to receive a sample from a collection site of a subject; (b) a sample processing system comprising a next generation sequencing platform configured to receive the sample container, associate the sample container with the subject, and generate a microbiome feature dataset based upon sequencing the nucleic acid content of a microorganism portion of the sample; and (c) a computing device, which during operation of the system: (i) communicates with the next generation sequencing platform of the sample processing system and identifies a set of microorganisms represented in the microorganism portion based upon performance of a mapping operation on portions of the microbiome sequence dataset; (ii) receives microbial sequence data comprising a microbiome feature dataset from an aggregate set of samples from a population of pregnant or breastfeeding female individuals; (iii) receives a supplementary dataset informative of at least one characteristic associated with the population of individuals and the subject; (iv) inputs the received datasets into a machine learning model to generate a dietary regimen that promotes eubiosis and thereby promotes maternal and/or fetal wellbeing; and (v) transmits the dietary regimen to the subject.

In some embodiments, the aggregate set of samples comprises fecal, oral, salivary, buccal, skin, and/or vaginal microbial samples.

In some embodiments, the subject is a human subject.

In some embodiments, the aggregate set of samples comprises samples taken over a range of gestational statuses or times post-partum.

In some embodiments, the set of primers comprises primers that amplify 16S sequences or specific genes.

In some embodiments, the microbiome feature dataset comprises microbial taxa and the relative abundance of microbial taxa.

In some embodiments, the at least one characteristic associated with the population of individuals and the subject is selected from the group consisting of: gestational status, different dietary habits (e.g., omnivorous, vegetarian, vegan, sugar consumption, acid consumption, fiber consumption, protein consumption, carbohydrate consumption, fat consumption, etc.), demographics (e.g., ages, marital statuses, ethnicities, nationalities, socioeconomic statuses, sexual orientations, etc.), different health conditions (e.g., health and disease states), different living situations (e.g., living alone, living with pets, living with a significant other, living with children, etc.), different behavioral tendencies (e.g., levels of physical activity, drug use, alcohol use, etc.), different levels of mobility (e.g., related to distance traveled within a given time period), and any other suitable trait that has an effect on microbiome composition.

In some embodiments, generating a dietary regimen comprises inputting the supplementary dataset and the microbiome feature dataset of the subject into a machine learning model trained on the supplementary dataset and the microbiome feature dataset of the population of individuals.

In some embodiments, the machine learning model outputs a dietary regimen based on the characterization of the subject's dysbiosis comprising varieties and quantities of foods that alter the dysbiosis towards a state of eubiosis.

In some embodiments, the dietary regimen comprises recommended intake of carbohydrates, fat, protein, and fiber; recommended meals; or discouraged meals.

In some embodiments, the output device is further configured to provide an output reporting whether a scanned QR-coded food product adheres to the dietary regimen.

In some embodiments, the machine learning model comprises a multilayer perceptron, a linear regression, K-Nearest Neighbor, or a K-NN classifier.

In some embodiments, the population of individuals or the subject provides a plurality of samples at different times during pregnancy or breastfeeding.

In some embodiments, performance of the mapping operation on portions of the microbiome sequence dataset comprises performing quality filtering of reads of the microbiome sequence dataset, and identifying and removing human genome-derived sequences from the microbiome sequence dataset.

In some embodiments, during operation, the computing device renders a graphic derived from the analysis at a display of an electronic device associated with the subject, upon accessing of a user account by the subject at the electronic device, wherein the graphic depicts a distribution of taxonomic groups of microorganisms present in the sample with comparisons to distributions from other groups of individuals.

### INCORPORATION BY REFERENCE

Each patent, publication, and non-patent literature cited in the application is hereby incorporated by reference in its entirety as if each was incorporated by reference individually.

### BRIEF DESCRIPTION OF THE DRAWINGS

FIG. 1 is a schematic showing an example system for implementing the methods described herein.

FIG. 2 is a diagram illustrating an example timeline for screening for Gestational Diabetes Mellitus (GDM) using microbiome screening kits of the disclosure.

FIG. 3 is an example instruction card for stool sample collection that can be provided to patients.

FIG. 4 is a user interface flowchart for participant submission of dietary data during a clinical trial.

FIG. 5 is a schematic showing an example workflow for processing microbiome sequence data and generation of a microbiome profile.

FIG. 6 is an example smartphone interface for viewing microbiome data and dietary recommendations generated by the methods disclosed herein.

FIG. 7 is a non-limiting example of microbiome data generated by the methods disclosed herein.

FIG. 8 is a non-limiting example of microbiome data generated by the methods disclosed herein.

FIG. 9 is a non-limiting example of a microbiome heatmap based on data generated by the methods disclosed herein.

FIG. 10 is a non-limiting example of a microbiome heatmap based on data generated by the methods disclosed herein.

FIG. 11 is an example workflow for characterizing the microbiome.

FIG. 12 is an example workflow for characterizing the microbiome.

FIG. 13 is an example computational workflow for characterizing the microbiome.

FIG. 14 is an example taxonomy classification and phylogeny analysis.

FIG. 15 is an example computer system described herein.

### DETAILED DESCRIPTION

Described herein are methods and systems of promoting eubiosis or treating dysbiosis in a subject in need thereof. In some embodiments, the subject is a female within about a year of giving birth. In some embodiments, the subject is a pregnant or breastfeeding woman. In some embodiments, the subject is an infant. Also described herein are sampling kits and methods of providing said kits to subjects of interest.

### Gut Microbiome and Metagenomic Sequencing

The gut microbiome comprises the collection of all living microbes, including eukaryotes, bacteria, archaea, viruses, and fungi, e.g., inside the gastrointestinal tract. The human microbiome is the collection of all microbes that live in and on human individuals.

The microbiome is a component of human health. These microbes are generally not harmful to humans, and can be essential for maintaining health. For example, the microbiome produces some vitamins that the human body does not have the genes to produce. The microbiome can also break down the food to extract nutrients. A healthy gut microbiota is involved in energy extraction from dietary components, regulation of components of the immune system, vitamin synthesis, energy metabolism, regulation of the insulin response, and colonization resistance, i.e., protection against colonization by gastrointestinal pathogens. A number of major gastrointestinal bacteria groups exist, including the genera Bacteroides, Bifidobacterium, and Bacillus and proteolytic cocci.

Regulation of a healthy microbiome, e.g., through the diet, becomes especially important during pregnancy. A mother's gut microbiome can change dramatically throughout pregnancy. However, current sources of nutritional advice for pregnant women are generic and outdated. Therefore, personalized nutrition recommendations for pregnant women that take into account microbiome composition and other characteristics are needed.

The microbiome can be an important contributor to human health and disease. Various methods can be used to characterize the microbiome of a subject to provide a microbiome profile or composition specific to the subject. Non-limiting examples of such methods include genomic sequencing, microarrays, RNA sequencing (e.g., RNA-Seq), matrix-assisted laser desorption/ionization time of flight (MALDI-TOF) mass spectrometry, fluorescence in situ hybridization-flow cytometry (FISH-FCM), and quantitative polymerase chain reaction (qPCR).

High-throughput sequencing can be used to elucidate the gut microbial population. Based on sequencing data, relationships between the microbiome to health and disease can be elucidated. Next-generation sequencing (NGS), also called high-throughput sequencing, is a massively parallel sequencing method that allows researchers to inexpensively determine entire or partial genomic sequences in a short period of time. NGS can exhibit significantly improved scalability compared to other sequencing methods. NGS requires that each individual sequence be read multiple times to have 99% coverage aligned to reference bases. The sequencing coverage level can determine whether variant discovery can be made with a certain degree of confidence at particular base positions. At higher levels of coverage, each base is covered by a greater number of aligned sequence reads, to give a confidence level for each nucleotide. Each nucleotide is recommended to be reported within an assembled sequence an average of 30× times (i.e., 30 reads). As the read depth level increases, the sequencing information has a higher confidence level. High-throughput sequencing technologies have become essential in genomics and metagenomics. By overcoming limitations in sequencing depth, NGS has led to a dramatic increase in the analytical complexity of samples that can be analyzed. Applying modern high-throughput sequencing, the entire microbial community can be profiled to reveal an extensive diversity of genes and organisms.

In one example, NGS is based upon the idea of sequencing by synthesis. Each time a base is incorporated, the ION TORRENT Personal Genome Machine® (PGM) measures a change in pH. Such NGS methods have advanced understanding of the complexity and diversity of the gut microbial communities within and between individuals. Sequencing the gut microbiome of humans can contribute to the development of a model system to study and detect the effects of drugs or natural products on the microbial flora.

The link between sequence, microorganisms, and the human host can be essential to understanding the molecular mechanisms of evolution and for the design of nucleic acids, proteins, and genes with specific properties. However, the relationship between sequence and microbiome or organismal phenotypes can be difficult to determine due to the complex relationship between sequence, nucleic acids and microbiome activity, and organismal physiology.

In one aspect, described herein is a method for diagnosing and treating a dysbiosis in a pregnant or breastfeeding female subject, the method comprising: (a) receiving microbial sequence data comprising a microbiome feature dataset from an aggregate set of samples from a population of pregnant or breastfeeding female individuals; (b) receiving a sample from the subject; (c) determining microorganism nucleic acid sequences for the subject sample, comprising: (i) fragmenting nucleic acid material of the sample, and (ii) amplifying the fragmented nucleic acid material with a set of primers; (d) determining an alignment of the microorganism nucleic acid sequences to reference nucleic acid sequences in a database; (e) generating a microbiome feature dataset for the subject based upon the alignments; (f) generating a characterization of the subject's dysbiosis based upon: (i) a supplementary dataset informative of at least one characteristic associated with the population of individuals and the subject, and (ii) the microbiome feature datasets of the population of individuals and the subject; (g) based upon the characterization, generating a dietary regimen for correcting the dysbiosis; and (h) at an output device associated with the subject, providing the dietary regimen to the subject with the dysbiosis based upon the characterization.

In some embodiments, the method comprises receiving microbial sequence data comprising a microbiome feature dataset from an aggregate set of samples from a population of pregnant or breastfeeding female individuals. In some embodiments, the method comprises receiving microbial sequence data comprising a microbiome feature dataset from an aggregate set of samples from a population of infant individuals. In some embodiments, the population of individuals comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20, at least 25, at least 30, at least 35, at least 40, at least 45, at least 50, at least 55, at least 60, at least 65, at least 70, at least 75, at least 80, at least 85, at least 90, at least 95, at least 100, at least 150, or at least 200 individuals.

In some embodiments, the method comprises receiving a sample from a subject. The sample can be taken or isolated from a biological organism, e.g., a blood or plasma sample from a subject. In some embodiments, the present invention encompasses several examples of a biological sample. In some embodiments, the biological sample is cells, or tissue, or peripheral blood, or bodily fluid. In some embodiments, the aggregate set of samples comprises fecal, oral, salivary, buccal, skin, and/or vaginal microbial samples. Non-limiting examples of biological samples include a biopsy, biofluid sample; human milk; blood; serum; plasma; urine; semen; mucus; tissue biopsy; organ biopsy; placenta; synovial fluid; bile fluid; cerebrospinal fluid; mucosal secretion; effusion; sweat; saliva; tissue sample; and combinations thereof.

In some embodiments, the aggregate set of samples comprises samples taken over a range of gestational statuses or times post-partum. In some embodiments, the aggregate set of samples comprises samples taken over a range of infant ages. In some embodiments, the plurality of samples is provided during at the first trimester, second trimester, or third trimester. In some embodiments, the samples are provided on bimonthly, monthly, twice monthly, weekly, daily, twice daily, three times daily or four or more times daily over a period of 1 week, 2 weeks, 3 weeks, 4 weeks, 1 month, 2 months, 3 months, 4 months, 5 months, or 6 months, or more.

The sample can be obtained by removing or extracting a sample from a subject, but can also be accomplished by using a previously isolated sample (e.g., isolated at a prior time point by the same or another person). In some embodiments, the methods and systems described herein can further comprise a step of obtaining or having obtained a sample from a subject.

In some embodiments, the present disclosure provides a method for identifying a presence or susceptibility of a pregnancy-related condition in a subject, the method comprising a) assaying microbial nucleic acids from a biological sample of the subject, thereby generating a microbiome profile of the subject; and b) identifying the presence or susceptibility of the pregnancy-related condition in the subject based on the microbiome profile of the subject.

In some embodiments, the pregnancy-related condition is a dysbiotic state, gestational diabetes, glucose intolerance, preeclampsia, eclampsia, hyperemesis gravidarum, anemia, morning sickness, constipation, acid reflux, bacterial vaginosis, thrush, metabolic syndrome, kidney infection, neonatal infection, mastitis, preterm birth, ectopic pregnancy, spontaneous abortion, stillbirth, post-partum depression, hemorrhage or excessive bleeding during delivery, premature rupture of membrane, placenta previa, intrauterine/fetal growth restriction, macrosomia, or HELLP syndrome.

In some embodiments, the pregnancy-related condition is gestational diabetes. In some embodiments, the gestational diabetes is type A1 gestational diabetes or type A2 gestational diabetes.

In some embodiments, the pregnancy-related condition comprises a fasting blood glucose level of at least about 90 mg/dL, at least about 95 mg/dL, at least about 100 mg/dL, or at least about 105 mg/dL. In some embodiments, the pregnancy-related condition comprises a blood glucose level of at least about 130 mg/dL, at least about 135 mg/dL, at least about 140 mg/dL, or at least about 145 mg/dL, wherein the blood glucose level is measured about 1 hour after consumption by the subject of a solution comprising about 50 g glucose, wherein the solution is consumed by the subject after fasting for at least about 8 hours. In some embodiments, the pregnancy-related condition comprises a blood glucose level of at least about 170 mg/dL, at least about 175 mg/dL, at least about 180 mg/dL, or at least about 185 mg/dL, wherein the blood glucose level is measured about 1 hour after consumption by the subject of a solution comprising about 75 g glucose, wherein the solution is consumed by the subject after fasting for at least about 8 hours. In some embodiments, the pregnancy-related condition comprises a blood glucose level of at least about 145 mg/dL, at least about 150 mg/dL, at least about 155 mg/dL, or at least about 160 mg/dL, wherein the blood glucose level is measured about 2 hours after consumption by the subject of a solution comprising about 75 g glucose, wherein the solution is consumed by the subject after fasting for at least about 8 hours. In some embodiments, the subject is from 24 to 28 weeks pregnant. In some embodiments, the subject has not been previously diagnosed with diabetes.

In some embodiments, the pregnancy-related condition comprises:
(a) a first blood glucose level of at least about 125 mg/dL, at least about 130 mg/dL, at least about 135 mg/dL, at least about 140 mg/dL, or at least about 145 mg/dL, wherein the first blood glucose level is measured about 1 hour after consumption by the subject of a first solution comprising about 50 g glucose, wherein the first solution is consumed by the subject upon fasting for at least about 8 hours; and
(b) at least two of:
   (i) a second blood glucose level of at least about 90 mg/dL, at least about 95 mg/dL, or at least about 100 mg/dL, wherein the second blood glucose level is measured upon the subject fasting for at least about 8 hours;
   (ii) a third blood glucose level of at least about 175 mg/dL, at least about 180 mg/dL, at least about 185 mg/dL, or at least about 190 mg/dL, wherein the third blood glucose level is measured about 1 hour after consumption by the subject of a second solution comprising about 100 g glucose, wherein the second solution is consumed by the subject upon fasting for at least about 8 hours;
   (iii) a fourth blood glucose level of at least about 150 mg/dL, at least about 155 mg/dL, at least about 160 mg/dL, or at least about 165 mg/dL, wherein the fourth blood glucose level is measured about 2 hours after consumption by the subject of the second solution; and
   (iv) a fifth blood glucose level of at least about 135 mg/dL, at least about 140 mg/dL, at least about 145 mg/dL, or at least about 155 mg/dL, wherein the fifth blood glucose level is measured about 3 hours after consumption of the second solution by the subject.

A subject can be a human subject. In some embodiments, the subject is a female subject. In some embodiments, the subject is a female within about a year of giving birth. In some embodiments, the subject is of childbearing age. In some embodiments, the subject is a pubescent or post-pubescent female subject. As a non-limiting example, the subject is at least 15 years old, at least 16 years old, at least 17 years old, at least 18 years old, at least 19 years old, at least 20 years old, at least 21 years old, at least 22 years old, at least 23 years old, at least 24 years old, at least 25 years old, at least 26 years old, at least 17 years old, at least 28 years old, at least 29 years old, at least 30 years old, at least 31 years old, at least 32 years old, at least 33 years old, at least 34 years old, at least 35 years old, at least 36 years old, at least 37 years old, at least 38 years old, at least 39 years old, at least 40 years old, at least 41 years old, at least 42 years old, at least 43 years old, at least 44 years old, at least 45 years old, at least 46 years old, at least 47 years old, at least 48 years old, at least 49 years old, or at least 50 years old.

In some embodiments, the subject is a pregnant female subject. The subject can be from any stage of pregnancy. As a non-limiting example, the subject can be 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, or more weeks pregnant. In some embodiments, the subject is in the first trimester, second trimester, or third trimester of pregnancy. In some embodiments, the subject is pregnant with 1, 2, 3, 4, 5, or more embryos or fetuses. In some embodiments, the subject is pregnant with at least one female embryo or fetus. In some embodiments, the subject is pregnant with at least one male embryo or fetus.

In some embodiments, the subject is a breastfeeding, nursing, or lactating female subject. In some embodiments, a subject that has lactated or breastfed an infant at least once. In some embodiments, the subject is a post-partum female subject. As a non-limiting example, the subject can be 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or more weeks post-partum. Any such information about the subject or pregnancy, breastfeeding status, or post-partum status can be included as a queried characteristic.

In some embodiments, the subject is not pregnant. In some embodiments, the subject is preconception or trying to conceive, i.e., the subject is trying to become pregnant. Methods described herein can increase a likelihood for a subject to become pregnant.

In some embodiments, the subject is an infant. The infant subject can be a female or male infant. In some embodiments, the infant subject is 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks old. In some embodiments, the infant subject is 13 months, 14 months, 15 months, 16 months, 17 months, 18 months, 19 months, 20 months, 21 months, 22 months, 23 months, or at most 24 months old. In some embodiments, the infant subject is breastfed. In some embodiments, the infant subject is formula-fed. In some embodiments, the infant was born vaginally. In some embodiments, the infant was born via Caesarean section. Any such information about the infant subject can be included as a queried characteristic.

In some embodiments, the population of individuals or the subject provides a plurality of samples at different times during pregnancy or breastfeeding. As a non-limiting example, the population of individuals or the subject provides at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 samples at different times during pregnancy or breastfeeding. As a non-limiting example, the population of infant individuals or the infant subject provides at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 samples at different times during infancy.

In some embodiments, the subject is an individual associated with a pregnant subject, a breastfeeding subject, a post-partum subject, or an infant subject. As a non-limiting example, the subject can be a caretaker, a spouse, a significant other, or another family member.

A subject can be one who has been previously diagnosed with or identified as suffering from or having a condition in need of treatment (e.g., dysbiosis) or one or more complications related to such a condition. In some cases, such a subject has already undergone treatment for dysbiosis, or the one or more complications related to dysbiosis. Alternatively, a subject can also be one who has not been previously diagnosed as having dysbiosis or one or more complications related to dysbiosis. For example, a subject can be one who exhibits one or more risk factors for dysbiosis, or one or more complications related to dysbiosis or a subject who does not exhibit risk factors. A subject can be a subject having a condition, diagnosed as having a condition, or at risk of developing a condition.

Nucleic acids can be extracted from a biological sample for microbiome profiling. In some embodiments, a sample of a subject is a biological sample. Non-limiting examples of a biological sample is stool, feces, oral, saliva, buccal, vaginal tissue, vaginal fluid, blood, placental tissue, amniotic fluid, or skin. A biological sample can include one or more of stool, feces, oral, saliva, buccal, vaginal tissue, vaginal fluid, blood, placental tissue, amniotic fluid, or skin.

A dysbiosis can be an imbalance in types of microbiota that impact human health, to cause or make worse certain disease states and to upset the natural workings of the body. In some embodiments, a dysbiosis can occur in the microbiota of the gastrointestinal tract, skin, mouth, vagina, etc. The microbiota can modulate natural workings of the body, including but not limited to modulation of the immune system, intestinal maturation, production of short chain fatty acids, mucosal physiology, and the production of vitamins, including Vitamin K and biotin. When a dysbiosis exists, the dysbiosis can cause or worsen certain diseases. Non-limiting examples of dysbiosis-associated conditions include inflammation, bowel disease, chronic fatigue syndrome, obesity, diabetes, bacterial vaginitis related cancer, antibiotic resistance development, nonalcoholic steatohepatitis (NASH), sleep disorder, gastro esophageal reflux disease (GERD), atopic dermatitis, hypertension associated with obstructive sleep apnea, cardiovascular inflammation associated with obstructive sleep apnea, enteritis-related arthritis, and celiac disease. Dysbiosis-associated conditions also include irritable bowel syndrome, colitis, and colorectal cancer. In some embodiments, a condition in the subject is based on an enrichment or a deficiency of a bacterial species in the biological sample of the subject.

In some embodiments, the dysbiosis can be the relative or absolute abundance of specific species of microorganisms in the gastrointestinal tract of a non-healthy subject. In some embodiments, the non-healthy subject is a non-healthy pregnant, breastfeeding, post-partum, or infant subject. In some embodiments, the non-healthy subject is not pregnant. A non-healthy subject can be a subject having a dysbiosis-associated condition as described herein, diagnosed as having a dysbiosis-associated condition, or at risk of developing a dysbiosis-associated condition.

A eubiosis can be an optimal or otherwise healthy balance of microflora, e.g., in the gastrointestinal tract. In some embodiments, eubiosis can be the relative or absolute abundance of specific species of microorganisms (e.g., in the gastrointestinal tract) of a healthy subject. In some embodiments, the healthy subject is a healthy pregnant, breastfeeding, post-partum, or infant subject. In some embodiments, the healthy subject is not pregnant. A healthy can be a subject not having a dysbiosis-associated condition as described herein, not diagnosed as having a dysbiosis-associated condition, or not at risk of developing a dysbiosis-associated condition.

In some embodiments, the dysbiosis or eubiosis is specific to a pregnant subject. In some embodiments, the dysbiosis or eubiosis is specific to a breastfeeding or post-partum subject. In some embodiments of any of the aspects, the dysbiosis or eubiosis is specific to an infant subject.

During the course of pregnancy, changes occur in the microbiota composition at a variety of body sites including the gut, vagina, oral cavity, and placenta. Accordingly, described herein are methods and systems that can account for the normal changes to the maternal and/or fetal microbiome that occur at varying stages of gestation and/or lactation.

During gestation, the female body undergoes hormonal, immunological, and metabolic changes to support fetal growth and development. During this period, levels of secreted hormones (especially progesterone and estrogens) can rise dramatically, and major alterations in the immune response can occur. Changes to the immune response, or immune modulation, can be complex. On the one hand, some degree of immune suppression is needed to accept the growing fetus that bears a developing immune system. On the other hand, strict immunity must remain to protect the mother and fetus from infections. Pregnancy can be considered as a multi-stage process that includes inflammatory stages at implantation and parturition, and anti-inflammatory stages in mid-pregnancy when the fetus grows rapidly.

In many ways, the metabolic changes associated with pregnancy are similar to those that occur in the metabolic syndrome, including weight gain, elevated fasting blood-glucose levels, insulin resistance, glucose intolerance, low-grade inflammation, and changes in metabolic hormone levels.

In addition to the endocrine, metabolic, and immune alterations discussed above, noticeable changes occur in the microbiota at different body sites during pregnancy. Healthy pregnancy can be characterized by an increase in the bacterial load and profound alterations in the composition of gut microbiotas. In the first trimester of pregnancy, the gut microbial composition can be similar to that of healthy, non-pregnant women. However, from the first to the third trimester, the gut microbiota composition can change dramatically. These changes can be characterized by increased abundance of members of certain bacterial species and decreased abundance of members of other bacterial species. For example, an increased abundance of members of the Actinobacteria and Proteobacteria phyla can occur, as can a reduction in individual richness, e.g., alpha diversity. Levels of Faecalibacterium, a butyrate-producing bacterium with anti-inflammatory activities, which are depleted in metabolic syndrome patients, can be significantly decreased in the third trimester of pregnancy. Between-subject diversity (e.g., beta diversity) can be increased in the third trimester, and can coupled with weight gain, insulin insensitivity, and elevated levels of fecal cytokines that can be indicative of inflammation.

Microbial components can actively contribute to changes in host immunology and metabolism, which is characterized by changes seen in the metabolic syndrome. However, unlike metabolic syndrome, the bacterial composition and physical parameters from the third trimester of pregnancy can have positive and essential effects in the context of pregnancy. These parameters can contribute to a healthy pregnancy and appropriate fetal development. During pregnancy, weight gain, insulin insensitivity, and a shift to an inflammatory state can be necessary to support the growing fetus. Some mechanisms by which the gut microbiota plays a role in host weight gain during pregnancy can include enhanced absorption of glucose and fatty acids, increased fasting-induced adipocyte factor secretion, induction of catabolic pathways, and stimulation of the immune system.

The gut microbiota during pregnancy can be influenced not only by internal cues, but also by environmental factors, such as diet. The maternal diet prior to and during pregnancy can have an effect on gut microbiota. Female mice fed a high-fat diet in the periconceptional period and during gestation can demonstrate changes in the gut microbiota later in pregnancy, in contrast to those fed on a normal chow diet. An obese state can also be correlated to microbial composition during human gestation. Bacteroides and Staphylococcus can be significantly higher in the feces of overweight pregnant women, compared to those of normal weight. Additionally, in overweight and obese pregnant women, specific metabolic hormones including insulin, gastric inhibitory polypeptide (GIP), and adipokines can be correlated with alterations in bacterial abundance. This phenomenon reinforces a connection between the microbiota and metabolic hormones in pregnancy. Pre-pregnancy maternal body mass index (BMI) can be correlated to neonatal gut microbiota composition in vaginally delivered offspring, but not Cesarean section (C-section) delivered offspring. Overall, significant alterations in gut microbiota during pregnancy, can be correlated with initial weight and diet, weight gain, inflammation, and metabolic parameters.

The gut microbiome of a pregnant, breastfeeding, post-partum, or infant subject can be shaped by diet and genetics. The typical Western diet includes excessively processed foods, dietary fats, and sugars. Such a diet can promote excess weight gain and a dysbiotic gut, and can be associated with adverse maternal and child health outcomes. Conversely, certain dietary nutrients, for example, low-fat protein (e.g., from beans, skinless chicken, or lean beef), organic proteins and produce (which reduce exposure to dietary antibiotics and pesticides), unsaturated fatty acids (e.g., in canola oil, olive oil, flaxseeds, and salmon), whole grains, and certain strains of probiotics can promote a healthy gut microbiome, enhance intestinal integrity, and reduce excessive systemic inflammation. Short chain fatty acids (SCFA), such as acetate, propionate, and butyrate, are byproducts of fiber fermentation called prebiotics, and can serve as an energy source to intestinal epithelial cells and enhance the connections between these cells. SCFA production by the gut microbiota can be negatively correlated with body mass index. Increased maternal serum SCFA levels can positively influence metabolic changes seen in pregnancy, e.g., maternal weight gain, glucose metabolism, and levels of various metabolic hormones. In overweight and obese pregnant women, an abundance of butyrate-producing bacteria can be negatively associated with systolic and diastolic blood pressure and plasminogen activator inhibitor-1 (PAI-1) levels. Thus, increasing butyrate-producing bacteria can lower blood pressure in overweight and obese pregnant women. Omega-3 long-chain polyunsaturated fatty acids (PUFAs) can also provide protection of the intestinal wall through strengthening of cellular connections. Omega-3 supplementation in overweight and obese pregnant women can cause a significant reduction in the plasma inflammatory marker, CRP. Such a supplementation can be an effective strategy to reduce the low-grade inflammation and obstetric risks suffered by overweight and obese women or women with gestational diabetes.

Maternal diet during pregnancy can also be related to the infant stool microbiome in a delivery mode-dependent manner. As a non-limiting example, an increased abundance of Bifidobacterium, Streptococcus, Clostridium, and Bacteroides can occur in vaginally delivered infants. Maternal fruit intake can also be associated with infant gut microbial community structure. Increased fruit intake can be associated with increased odds of a vaginally born infant having high Streptococcus and/or Clostridium levels. Infants delivered by C-section can be characterized by a high abundance of Bifidobacterium, high abundance of Clostridium, low abundance of Streptococcus, low abundance of Ruminococcus genera, and high abundance of the family Enterobacteriaceae. Maternal dairy intake can be also associated with increased odds of infants having high Clostridium in infants born by C-section.

Antibiotics administered during pregnancy can have significant effects on the microbiome. For example, use of category B antibiotics (e.g., azithromycin, amoxicillin, and cefaclor) during pregnancy can increase relative abundance of Proteobacteria and Enterobacter, while reducing the relative abundance of Firmicutes and Lactobacillus in fecal matter. Moreover, antibiotics during pregnancy can significantly reduce bacterial diversity and promote weight gain. Maternal antibiotic treatment during pregnancy and can also reduce adaptive antiviral immune responses in the infant. This observation suggests a broad immune effect on the offspring. Further, non-absorbable antibiotics given to a pregnant subject can alter offspring behavior in a manner by which the offspring exhibits low locomotor activity at 4 weeks of age, and less exploratory behavior in central regions at both 4 and 8 weeks of age.

Maternal microbiome composition during pregnancy can impact the offspring in terms of weight gain, immunity, and infant health. Oligofructose prebiotic treatment during pregnancy and lactation can alter the offspring's cecal microbiome and prevent increased adiposity in both the mother and the offspring. Administering probiotics to a pregnant women 14 days before C-section can modulate the infant microbiota. This finding suggests that maternal effect on the offspring's microbiota can occur early. A correlation can exist between maternal probiotics and changes in expression of Toll-like receptor genes in the placenta and infant meconium. Moreover, microbial exposure during pregnancy can be important for preventing allergic disease in the offspring. The mechanisms include immune-regulated epigenetic imprinting and bacterial translocation during pregnancy from the mother to the offspring. This mechanism trains the offspring's immune system to respond appropriately to pathogens and commensals after birth. Accordingly, mothers with allergic disease and atopic sensitization that received probiotics during the last 2 months of pregnancy and the first 2 months of lactation can have a reduced risk for eczema in the offspring. Maternal microbiota can shape the offspring's immune system. Mouse pups of colonized GF mothers can show increased numbers of intestinal group 3 innate lymphoid cells, F4/80⁺CD11c⁺ mononuclear cells, and increased epithelial antibacterial peptide gene expression.

During pregnancy, inflammatory states can vary from higher inflammation at implantation and in labor to lower levels of inflammation in mid-pregnancy. For example, the placental bed, which protects the fetus from rejection early in pregnancy, can exhibit anti-inflammatory properties. As the pregnancy advances, the mucosal surfaces of the gut and other tissues can experience a low-grade inflammation with rising levels of pro-inflammatory cytokines and white cells. When conditions such as maternal obesity, gestational diabetes, or a leaky gut shift the maternal inflammatory state from a physiologic level to an excessive level, vascular dysfunction of the placental tissue can develop. Vascular dysfunction can lead to deleterious effects such as fetal growth restriction and preeclampsia. Gut microbes found in amniotic fluid of women who experienced preterm premature rupture of membranes suggest that the gut microbiome plays a role in intrauterine infections. Mechanisms of microbial translocation can be induced by lipopolysaccharides (LPS) from gram negative bacteria that increase inflammatory mediators and prostaglandin production, via ascending inoculation through the vagina, or by hematogenous spread of microbes from a leaky gut to the placenta or uterus.

The postpartum period can also be characterized by significant changes in the microbiota. In some cases, the mothers' microbiotas do not return to baseline for at least a month after birth. The duration of the postpartum transition period is unclear and a complete return to baseline microbial populations may not occur. Since the postpartum period is also associated with dramatic hormonal changes, including a significant decrease in progesterone and estrogen levels, the hormonal changes can have direct effects on the microbiome.

In some embodiments, the methods or systems described herein comprise analyzing the vaginal, oral, and/or placental microbiomes of the pregnant, breastfeeding, and/or infant subject. In some embodiments, the methods or systems described herein comprise analyzing the vaginal, oral, and/or placental microbiomes of a non-pregnant subject. The human vaginal microbiota is a key component in the defense system against microbial and viral infections and can confer protection against disease. The vaginal microbiome is dominated by many species including Lactobacillus and members of the Clostridiales, Bacteriodales, and Actinomycetales. Within the Lactobacillus genus, the most frequently isolated species are: Lactobacillus gasseri, Lactobacillus crispatus, Lactobacillus jensenii, and Lactobacillus iners, each of which can promote various aspects of vaginal health. Specifically, these lactic acid producing bacteria can create a barrier against pathogen invasion by maintaining a low pH (<4.5) and by secreting metabolites that play an important role in inhibition of bacterial and viral infection in the urogenital tract. Higher pH values, e.g., around 5.0, can be correlated with vaginosis.

As in the gut, the vaginal microbiome can undergo significant changes during pregnancy, including a significant decrease in overall diversity, increased stability (i.e., the community composition changes over time), and enrichment with Lactobacillus species. These changes can correlate with a decrease in the vaginal pH and an increase in vaginal secretions. Vaginal microbial compositions can differ according to gestational age, while the communities at the later stages of pregnancy can resemble those of the non-pregnant state. The dominant Lactobacillus species in pregnancy can vary according to ethnic group. For example, L. jensenii is predominantly observed in women of Asian and Caucasian ethnicity, whereas L. gasseri is absent in samples from Black women.

In characterizing the vaginal microbiota of pregnant and non-pregnant African-Americans, one of the differences is dominance of a single Lactobacillus species over others. This finding can be due to the fact that some Lactobacillus species have bactericidal activities against other species, ensuring their predominance and low variability, which may help in protection against infections during pregnancy.

Distinct microbiome differences can exist in the post-partum period in which vaginal communities are more similar to the gut microbiota communities, with Lactobacillus being replaced by various anaerobic bacteria, including Peptoniphilus, Prevotella, and Anaerococcus. These various alterations can persist for as long as a year after delivery. The postpartum vaginal microbiome can also be characterized by gradual depletion of Lactobacillus species, increased alpha-diversity, and enrichment of bacteria associated with vaginosis, such as Actinobacteria, in 40% of the subjects at 6 weeks after birth (as opposed to only 2% of the subjects during pregnancy).

The oral microbiome includes up to 600 diverse species including Streptococci, Lactobacilli, Staphylococci, Corynebacteria, etc. that reside in different microenvironments within the oral cavity (e.g., teeth, tongue, palates, etc.). When comparing the abundance of seven common bacterial species in the oral cavity of non-pregnant women, early pregnancy, mid-pregnancy, and late pregnancy, the total viable microbial counts in all stages of pregnancy can be higher than those of the non-pregnant women, especially in early pregnancy, and levels of the pathogenic bacteria Porphyromonas gingivalis and Aggregatibacter actinomycetemcomitans in the subgingival plaque, can be significantly higher during the early and middle stages of pregnancy, compared to the non-pregnant group. These results can be further reinforced in an additional study that shows higher levels of A. actinomycetemcomitans in the second and third trimesters of pregnancy compared to non-pregnant women. Additionally, Candida levels can be significantly higher during middle and late pregnancy compared to non-pregnant women, further demonstrating a higher prevalence of periodontal pathogens in pregnancy.

Progesterone and estrogen affect the microbiota during pregnancy. For example, estrogens enhance Candida infections. The overall immune state during pregnancy can lead to increased oral microbial load. Numerous correlations between oral disease and pregnancy complications and outcomes can be identified.

The placenta is one of the most poorly understood human organs, particularly with regard to the presence of microbes. Cases of placental contamination can be caused by an infection originating from the lower genital tract. However, the normal placenta can also be a source of bacteria.

Aerobic bacteria can be detected in the placenta without any histological evidence of chorioamnionitis. In the placental microbial community, a major phylum is Proteobacteria. Compared to that of all other organs, the composition of the placental microbiota is most similar to the oral microbiota, which includes species such as Prevotella tannerae and Neisseria. The similarity between the oral and placental microbiota suggests that bacteria pass from the oral cavity to the placenta. This passage can explain the many observations of women with periodontal disease that have an increased risk of pregnancy complications. Additionally, several studies using cultivation and PCR techniques identified microbes in amniotic fluid and umbilical cord blood in healthy asymptomatic women, and in those with pregnancy complications. The microbial density present in the placenta can be very low and can require precise verification in all cases that research results are not due to contamination.

In some embodiments, nucleic acid is isolated from the sample. Nucleic acid, e.g., deoxyribonucleic acid (DNA) and ribonucleic acid (RNA), molecules can be isolated from a particular biological sample using any of a number of procedures. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction can be used to obtain nucleic acid from blood.

In some embodiments, the method comprises determining microorganism nucleic acid sequences for the subject sample. In some embodiments, determining microorganism nucleic acid sequences for the subject sample comprises: (i) fragmenting nucleic acid material of the sample, and (ii) amplifying the fragmented nucleic acid material with a set of primers.

In some embodiments, the level of or identity of a nucleic acid can be determined by a quantitative sequencing technology, e.g., a quantitative next-generation sequencing technology. Briefly, a sample obtained from a subject can be contacted with one or more primers which specifically hybridize to a single-strand nucleic acid sequence flanking the target gene sequence and a complementary strand is synthesized. In some next-generation technologies, an adaptor (double or single-stranded) is ligated to nucleic acid molecules in the sample and synthesis proceeds from the adaptor or adaptor compatible primers. In some third-generation technologies, the sequence can be determined, e.g., by determining the location and pattern of the hybridization of probes, or measuring one or more characteristics of a single molecule while passing through a sensor (e.g., the modulation of an electrical field as a nucleic acid molecule passes through a nanopore). Non-limiting examples of methods of sequencing include Sanger sequencing (i.e., dideoxy chain termination), Roche® 454 sequencing, sequencing by oligonucleotide ligation and detection (SOLiD) sequencing, polony sequencing, Illumina® sequencing, Ion Torrent sequencing, sequencing by hybridization, nanopore sequencing, HelioScope sequencing, single molecule real time sequencing, and RNAP sequencing.

In some embodiments, the set of primers comprises primers that amplify variable regions of 16S rRNA sequences, e.g., the 16S-IST-23S region. In some embodiments, the set of primers comprises primers that amplify variable regions of 23S rRNA sequences. In some embodiments, the set of primers comprises primers that amplify specific genes. In some embodiments, the sequence platform performs metagenomic sequencing, e.g., of DNA samples. Metagenomics is the study of the functional genomes of microbial communities while 16S sequencing offers a phylogenetic survey on the diversity of a single ribosomal gene, 16S rRNA product. In some embodiments, the sequencing platform performs whole genome sequencing, e.g., of DNA samples. In some embodiments, the method comprises sequencing an internal transcribed spacer between 16S and 23S rRNA genes.

In some embodiments, the sequencing platform performs metatranscriptomic sequencing, e.g., of RNA samples, to profile the gene expression of complex microbial communities. Microbiome profiles can inform differential expression or levels of bacterial strains. For example, microbiome profiles can indicate enriched strains for a given pregnancy or non-pregnancy state. While metagenomics focuses on studying the genomic content and identifying which microbes are present within a community, metatranscriptomics can also be used to study the diversity of the active genes within such community, to quantify expression levels and to monitor how these levels change in different conditions (e.g., physiological vs. pathological conditions in an organism). Metatranscriptomics can provide information about differences in the active functions of microbial communities, which appear to be the same in terms of microbe composition.

In some embodiments, the method comprises determining an alignment of the microorganism nucleic acid sequences to reference nucleic acid sequences in a database. Performing the sequencing analysis operation with identification of microbiome-derived sequences can include mapping of sequence data from sample processing to a subject reference genome (e.g., provided by the Genome Reference Consortium) to remove subject genome-derived sequences. Unidentified sequences remaining after mapping of sequence data to the subject reference genome can then be further clustered into operational taxonomic units (OTUs) based upon sequence similarity and/or reference-based approaches (e.g., using VAMPS, using MG-RAST, using QIIME databases), aligned (e.g., using a genome hashing approach, using a Needleman-Wunsch algorithm, using a Smith-Waterman algorithm), and mapped to reference bacterial genomes (e.g., provided by the National Center for Biotechnology Information), using an alignment algorithm (e.g., Basic Local Alignment Search Tool, FPGA accelerated alignment tool, BWT-indexing with BWA, BWT-indexing with SOAP, BWT-indexing with Bowtie, etc.). Mapping of unidentified sequences can additionally or alternatively include mapping to reference archaeal genomes, viral genomes and/or eukaryotic genomes. Furthermore, mapping of taxa can be performed in relation to existing databases, and/or in relation to custom-generated databases. In some embodiments, the performance of the mapping operation on portions of the microbiome sequence dataset comprises performing quality filtering of reads of the microbiome sequence dataset, and identifying and removing human genome-derived sequences from the microbiome sequence dataset.

In some embodiments, the method comprises generating a microbiome feature dataset for the subject based upon the alignments. In some embodiments, the microbiome feature dataset comprises microbial taxa and the relative abundance of microbial taxa. Furthermore, the microbiome feature dataset can comprise additional functional features associated with one or more of: prokaryotic clusters of orthologous groups of proteins (COGs); eukaryotic clusters of orthologous groups of proteins (KOGs); any other suitable type of gene product; an RNA processing and modification functional classification; a chromatin structure and dynamics functional classification; an energy production and conversion functional classification; a cell cycle control and mitosis functional classification; an amino acid metabolism and transport functional classification; a nucleotide metabolism and transport functional classification; a carbohydrate metabolism and transport functional classification; a coenzyme metabolism functional classification; a lipid metabolism functional classification; a translation functional classification; a transcription functional classification; a replication and repair functional classification; a cell wall/membrane/envelop biogenesis functional classification; a cell motility functional classification; a post-translational modification, protein turnover, and chaperone functions functional classification; an inorganic ion transport and metabolism functional classification; a secondary metabolites biosynthesis, transport and catabolism functional classification; a signal transduction functional classification; an intracellular trafficking and secretion functional classification; a nuclear structure functional classification; a cytoskeleton functional classification; a general functional prediction only functional classification; and a function unknown functional classification; and any other suitable functional classification.

Additionally or alternatively, the microbiome feature dataset can comprise additional functional features associated with one or more of: systems information (e.g., pathway maps for cellular and organismal functions, modules or functional units of genes, hierarchical classifications of biological entities); genomic information (e.g., complete genomes, genes and proteins in the complete genomes, orthologue groups of genes in the complete genomes); chemical information (e.g., chemical compounds and glycans, chemical reactions, enzyme nomenclature); health information (e.g., human diseases, approved drugs, crude drugs and health-related substances); metabolism pathway maps; genetic information processing (e.g., transcription, translation, replication and repair, etc.) pathway maps; environmental information processing (e.g., membrane transport, signal transduction, etc.) pathway maps; cellular processes (e.g., cell growth, cell death, cell membrane functions, etc.) pathway maps; organismal systems (e.g., immune system, endocrine system, nervous system, etc.) pathway maps; human disease pathway maps; drug development pathway maps; and any other suitable pathway map.

In some embodiments, generating the microbiome feature dataset can comprise performing a search of one or more databases, such as the Kyoto Encyclopedia of Genes and Genomes (KEGG) and/or the Clusters of Orthologous Groups (COGs) database managed by the National Center for Biotechnology Information (NCBI). Searching can be performed based upon results of generation of the microbiome feature dataset from one or more sets of aggregate biological samples. Searching can additionally or alternatively be performed according to any other suitable filters. In specific examples, generating the microbiome feature dataset can include extracting functional features, based on the microbiome feature dataset, from a KEGG database resource and a COG database resource; however, generating the microbiome feature dataset can comprise extracting functional features in any other suitable manner.

Upon identification of represented groups of microorganisms of the microbiome associated with a biological sample and/or identification of functional aspects (e.g., functions associated with the microbiome components of the biological samples), generating features derived from compositional and/or functional aspects of the microbiome associated with the aggregate set of biological samples can be performed.

In some embodiments, the method comprises generating a characterization of the subject's dysbiosis. In some embodiments, the characterization is based upon: (i) a supplementary dataset informative of at least one characteristic associated with the population of individuals and the subject, and/or (ii) the microbiome feature datasets of the population of individuals and the subject.

In some embodiments, the at least one characteristic associated with the population of individuals and the subject is selected from the group consisting of: gestational status, different dietary habits (e.g., omnivorous, vegetarian, vegan, sugar consumption, acid consumption, fiber consumption, protein consumption, carbohydrate consumption, fat consumption, etc.), demographics (e.g., ages, marital statuses, ethnicities, nationalities, socioeconomic statuses, sexual orientations, etc.), different health conditions (e.g., health and disease states), different living situations (e.g., living alone, living with pets, living with a significant other, living with children, etc.), different behavioral tendencies (e.g., levels of physical activity, drug use, alcohol use, tobacco use, etc.), different levels of mobility (e.g., related to distance traveled within a given time period), and any other suitable trait that has an effect on microbiome composition.

Additional non-limiting examples of characteristics include characteristics associated with pregnancy (e.g., number of previous pregnancies or live births; singleton vs. multiple pregnancy; number of previous C-sections or vaginal births; gestational diabetes status, preeclampsia status, Rh status; birth plans, etc.), breastfeeding (e.g., length of time breastfeeding, amount of milk produced per session, etc.), or infancy (e.g., breastfeeding vs. solid food status, developmental milestones, etc.).

In some embodiments, the supplementary dataset and/or microbiome feature dataset further comprises data derived from analysis of human milk samples. In some embodiments, the supplementary dataset comprises data such as the levels and types of human milk oligosaccharides (HMOs) as measured in a breast milk sample from a lactating subject. In some embodiments, the microbiome feature dataset comprises microbiome data from human milk samples. Human milk represents a continuous supply of probiotics (e.g., HMOs) and beneficial bacteria to the infant gut. The probiotics and bacteria contribute to the maturation of the digestive and immune functions in the developing infant. Diet can alter the oligosaccharide and microbiome composition of human milk.

In some embodiments, the supplementary dataset comprises data comprising caloric, carbohydrate, fat, protein, and fiber contents of foods. In some embodiments, such nutritional content data is acquired through applicable testing. In some embodiments, nutritional content data is acquired from a database or other publication. Non-limiting examples of sources of nutritional content data include the U.S. Department of Agriculture food database; the World Health Organization pregnancy nutrition database; American Dietetic Association recommendations for nutrition and lifestyle for a healthy pregnancy outcome; and the European Society for Clinical Nutrition and Metabolism dietary fat intake recommendations for pregnant and lactating women.

### Machine Learning and Artificial Intelligence

Machine Learning is a subset of the broader Artificial Intelligence field. Machine learning involves making computers able to perform specific tasks without being programmed explicitly. Specifically, machine learning can be divided into three main categories depending on how the computer learns: supervised, unsupervised, and semi-supervised learning. Supervised learning needs a training set of correctly labeled data, which is used by the algorithm to learn, penalizing every prediction that is different from the expected one. Unsupervised learning works with data that is not classified and can be used to find similarities and to group data. Semi-supervised learning takes a middle ground and uses a small amount of labeled data bolstering a larger set of unlabeled data. Machine learning can also comprise reinforcement learning, which needs no input training data and instead reacts with an environment through an agent, which evaluates the cost of every possible action. Among the many machine learning algorithms, K-Nearest Neighbor determines the result depending on the k nearest points to the one given as input.

In some embodiments, the method comprises based upon the characterization, generating a dietary regimen for correcting the dysbiosis. In some embodiments, generating a dietary regimen comprises inputting the supplementary dataset and the microbiome feature dataset of the subject into a machine learning model. In some embodiments, the machine learning model is trained on the supplementary dataset and/or the microbiome feature dataset of the population of individuals.

In some embodiments, the machine learning model is trained on microbiome sequencing data from subjects having the pregnancy-related condition and subjects not having the pregnancy-related condition, microbiome sequencing data from pregnant subjects having the pregnancy-related condition and pregnant subjects not having the pregnancy-related condition, nutritional habit data from subjects having the pregnancy-related condition and subjects not having the pregnancy-related condition, nutritional habit data from pregnant subjects having the pregnancy-related condition and pregnant subjects not having the pregnancy-related condition, blood glucose level data from subjects having the pregnancy-related condition and subjects not having the pregnancy-related condition, and/or blood glucose level data from pregnant subjects having the pregnancy-related condition and pregnant subjects not having the pregnancy-related condition.

In some embodiments, the machine learning model comprises multilevel mixed-effects regression and/or gradient boosting machine. As a non-limiting example, the machine learning model comprises a multilevel mixed-effects regression model where fixed effects represent measured properties and random effects account for further variation. The inclusion of random effects captures individual variation due to unobserved factors (unknown properties of the individual, meal, or measurement devices) that can affect the outcome. In some embodiments, the machine learning model comprises a gradient boosting machine, e.g., for maximizing predictive accuracy. In some embodiments, the machine learning model describes or predicts the relationships between nutrients, anthropometrics, and microbiome content.

In some embodiments, the machine learning model outputs a microbiome score for each subject. In some embodiments, the microbiome score comprises an aggregate assessment of overall ratios of active beneficial and harmful microbes. In some embodiments, the microbiome score comprises diversity metrics (e.g., measures of microbiome diversity). In some embodiments, the microbiome score is binary. In some embodiments, the microbiome score is numerical, e.g., ranging from 0 to 10. As a non-limiting example, the microbiome score can be determined by taking microbiome expression data (e.g., metatranscriptomics) as input, and applying a predetermined scoring algorithm. In some embodiments, metabolic and signaling pathway activities are scored using expression levels of genes encoding specific protein functions (e.g., KEGG mappings are used primarily), e.g., compared with a reference cohort of samples. In some embodiments, microbiome scores measure the quantity and expression levels of specific KEGG gene orthologs (KOs) selected due to their specific directional enzymatic roles, pathway topology, or significance, e.g., in the functional literature. In some embodiments, the more key genes expressed and the higher their expression levels, the higher the resulting score.

In some embodiments, the machine learning model comprises a multilayer perceptron, a linear regression, K-Nearest Neighbor, and/or a K-NN classifier. Variations of the method can additionally or alternatively utilize any other suitable algorithms in performing the characterization process. In variations, the algorithm(s) can be characterized by a learning style including any one or more of: supervised learning (e.g., using logistic regression, using back propagation neural networks), unsupervised learning (e.g., using an Apriori algorithm, using K-means clustering), semi-supervised learning, reinforcement learning (e.g., using a Q-learning algorithm, using temporal difference learning), and any other suitable learning style. Furthermore, the algorithm(s) can implement any one or more of: a regression algorithm (e.g., ordinary least squares, logistic regression, stepwise regression, multivariate adaptive regression splines, locally estimated scatterplot smoothing, etc.), an instance-based method (e.g., k-nearest neighbor, learning vector quantization, self-organizing map, etc.), a regularization method (e.g., ridge regression, least absolute shrinkage and selection operator, elastic net, etc.), a decision tree learning method (e.g., classification and regression tree, iterative dichotomiser 3, C4.5, chi-squared automatic interaction detection, decision stump, random forest, multivariate adaptive regression splines, gradient boosting machines, etc.), a Bayesian method (e.g., naïve Bayes, averaged one-dependence estimators, Bayesian belief network, etc.), a kernel method (e.g., a support vector machine, a radial basis function, a linear discriminate analysis, etc.), a clustering method (e.g., k-means clustering, expectation maximization, etc.), an associated rule learning algorithm (e.g., an Apriori algorithm, an Eclat algorithm, etc.), an artificial neural network model (e.g., a Perceptron method, a back-propagation method, a Hopfield network method, a self-organizing map method, a learning vector quantization method, etc.), a deep learning algorithm (e.g., a restricted Boltzmann machine, a deep belief network method, a convolution network method, a stacked auto-encoder method, etc.), a dimensionality reduction method (e.g., principal component analysis, partial lest squares regression, Sammon mapping, multidimensional scaling, projection pursuit, etc.), an ensemble method (e.g., boosting, bootstrapped aggregation, AdaBoost, stacked generalization, gradient boosting machine method, random forest method, etc.), and any suitable form of algorithm.

Additionally or alternatively, the dietary regimen can be derived in relation to identification of a normal or baseline microbiome composition and/or functional features, as assessed from a population of individuals (including, for example, a population of pregnant individuals, non-pregnant individuals, breastfeeding individuals, or infants) who are identified to be in good health. Upon identification of a subset of subjects of the population of individuals who are characterized to be in good health (e.g., using features of the characterization process), dietary regimens that modulate microbiome compositions and/or functional features toward those of subjects in good health can be generated. The methods as described herein can thus include identification of one or more baseline microbiome compositions and/or functional features (e.g., one baseline microbiome for each of a set of demographics), and potential dietary regimens that can shift microbiomes of subjects who are in a state of dysbiosis toward one of the identified baseline microbiome compositions and/or functional features. The dietary regimen can be generated and/or refined in any other suitable manner.

In some embodiments, the machine learning model outputs a dietary regimen based on the characterization of the subject's dysbiosis comprising varieties and quantities of foods that alter the dysbiosis towards a state of eubiosis (e.g., increase the microbiome score). In some embodiments, the dietary regimen comprises recommended intake of carbohydrates, fat, protein, and fiber; caloric restrictions; recommended meals; or discouraged meals. As a non-limiting example, the dietary regimen can comprise foods with at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 100% of the recommended daily intake values for carbohydrates, fat, protein, and/or fiber. In some embodiments, the recommended daily intake values for carbohydrates, fat, protein, and/or fiber can be specific to pregnant subjects, breastfeeding subjects, or infant subjects.

In some embodiments, the dietary regimen aids in digestive support. In some embodiments, the dietary regimen comprises foods rich in polyphenols, for example, micronutrients from certain plant-based foods that have high antioxidant levels. Non-limiting examples of foods rich in polyphenols include: cloves, peppermint (e.g., dried), star anise, cocoa powder, dark chocolate, berries, black chokeberry, highbush berries, blackberries, strawberries, red raspberries, black currants, plums, sweet cherries, apples, apple juice, pomegranate juice, beans, black beans, white beans, nuts, hazelnuts, walnuts, almonds, pecans, artichokes, chicory, red onions, spinach, soy, soy tempeh, soy flour, tofu, soy yogurt, soybean sprouts, black tea, and green tea.

In some embodiments, the dietary regimen comprises food comprising at least 15 mg, at least 20 mg, at least 25 mg, at least 30 mg, at least 35 mg, at least 40 mg, at least 45 mg, at least 50 mg, at least 100 mg, at least 200 mg, at least 300 mg, at least 400 mg, at least 500 mg, at least 600 mg, at least 700 mg, at least 800 mg, at least 900 mg, at least 1000 mg, at least 1100 mg, at least 1200 mg, at least 1300 mg, at least 1400 mg, at least 1500 mg, at least 1600 mg, at least 1700 mg, at least 1800 mg, at least 1900 mg, at least 2000 mg, at least 3000 mg, at least 4000 mg, at least 5000 mg, at least 6000 mg, at least 7000 mg, at least 8000 mg, at least 9000 mg, at least 10,000 mg, at least 11,000 mg, at least 12,000 mg, at least 13,000 mg, at least 14,000 mg, at least 15,000, or at least 15,500 mg polyphenols per 100 g of food (or per 100 mL of drink). In some embodiments, the dietary regimen comprises polyphenol supplements, e.g., as a powder or capsule.

In some embodiments, the dietary regimen comprises suggestions of ingredients that the subject should eat, which include, but are not limited to, onion, chives, leeks, rice, noodles, cranberry, basil, watermelon, garlic, or chicory root. In some embodiments, the dietary regimen comprises suggestions of ingredients that the subject should avoid, which include, but are not limited to, cardoon (e.g., globe artichoke), gourds, pumpkins, cucumbers, squash, luffa, melons, or rice noodle. In some embodiments, the dietary regimen comprises suggestions of ingredients that the subject should minimize or reduce, which include, but are not limited to, honey, hickory nuts, lams (e.g., Harungana madagascariensis Lam. ex Poir), oats, pomegranate, or sour cherries.

In some embodiments, the recommended dietary regimen is specific for pregnant subjects, lactating subjects, non-pregnant subjects, or infant subjects. As a non-limiting example, when determining the dietary regimen, the machine learning model takes into account the nutritional recommendations for pregnant subjects, lactating subjects, non-pregnant subjects, or infant subjects.

In some embodiments, the dietary regimen comprises recommended intake of probiotic bacteria (e.g., to increase the microbiome score). Non-limiting examples of probiotic bacteria include Lactobacillus species, Bifidobacterium species, Saccharomyces species, Lactobacillus rhamnosus, Bifidobacterium lactis, Lactobacillus plantarum, Lactobacillus acidophilus, Lactobacillus paracasei, Leuconostoc mesenteroides, Lactobacillus bulgaricus, Lactobacillus sasei, Lactobacillus salivarius, Pediococcus pentosaceus, Streptococcus thermophiles, Bacillus subtilis, Bacillus coagulans, and Enterococcus faecium. In some embodiments of any of the aspects, the probiotic bacteria are non-pathogenic.

In some embodiments, the dietary regimen comprises recommended intake of prebiotics. A prebiotic can be, for example, any substance or combination of substances that can be utilized as a nutrient by a microorganism, can induce the growth and/or activity of a microorganism, can induce the replication of a microorganism, can be utilized as an energy source by the microorganism, and/or can be utilized by the microorganism for the production of biomolecules (i.e., RNA, DNA, and/or proteins). Non-limiting examples of prebiotics include mucopolysaccharides, oligosaccharides, polysaccharides, amino acids, vitamins, nutrient precursors, harvested metabolic products of biological organisms, microbial lysates, lipids, and proteins.

In some embodiments, the method comprises providing the dietary regimen to the subject with the dysbiosis based upon the characterization. In some embodiments, the dietary regimen is provided at an output device associated with the subject. In some embodiments, the output device is a computing device or a personal electronic device, including but not limited to a smart phone, tablet, smart watch, or other smart device. In some embodiments, the output device is further configured to provide an output reporting whether a scanned (e.g., QR-coded) food product adheres to the dietary regimen.

### Systems

In some embodiments, described herein is a system for promoting maternal and/or fetal wellbeing, comprising: (a) a sampling kit including a sample container having a pre-process reagent component and configured to receive a sample from a collection site of a subject; (b) a sample processing system comprising a NGS platform configured to receive the sample container, associate the sample container with the subject, and generate a microbiome feature dataset based upon sequencing the nucleic acid content of a microorganism portion of the sample; and (c) a computing device, which during operation of the system: (i) communicates with the NGS platform of the sample processing system and identifies a set of microorganisms represented in the microorganism portion based upon performance of a mapping operation on portions of the microbiome sequence dataset; (ii) receives microbial sequence data comprising a microbiome feature dataset from an aggregate set of samples from a population of pregnant or breastfeeding female individuals; (iii) receives a supplementary dataset informative of at least one characteristic associated with the population of individuals and the subject; (iv) inputs the received datasets into a machine learning model to generate a dietary regimen that promotes eubiosis and thereby promotes maternal and/or fetal wellbeing; and (v) transmits the dietary regimen to the subject.

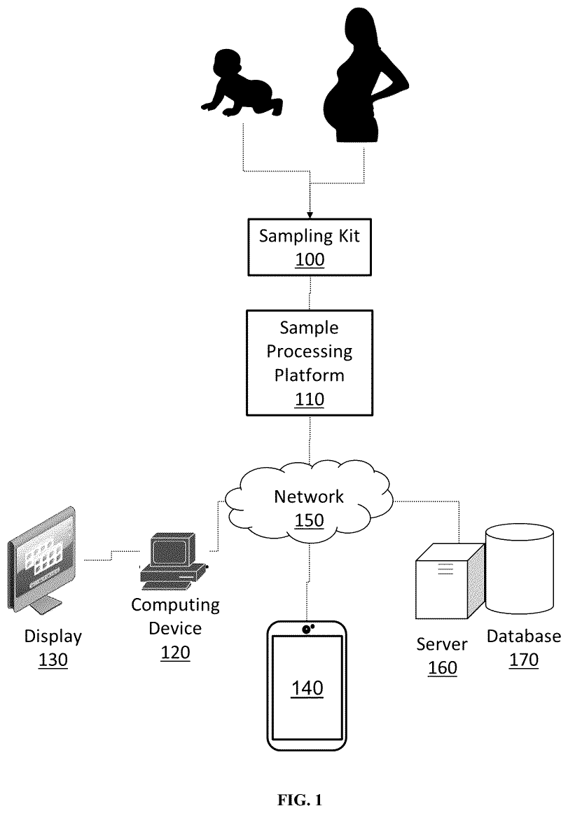

FIG. 1 illustrates an example overview of a system for implementing the methods described herein. In some embodiments, the system comprises a sampling kit 100. In some embodiments, the sampling kit 100 includes a sample container having a pre-process reagent component and configured to receive a sample from a collection site of a subject. In some embodiments, the subject is a pregnant woman, a breastfeeding woman, a non-pregnant woman, or an infant.

In the above variations and examples, samples can be taken from the bodies of subjects without facilitation by another entity (e.g., a caretaker associated with an individual, a health care professional, an automated or semi-automated sample collection apparatus, etc.), or can alternatively be taken from bodies of individuals with the assistance of another entity. In one example, wherein samples are taken from the bodies of subjects without facilitation by another entity in the sample extraction process, a sample-provision kit can be provided to a subject. In the example, the kit can include one or more swabs for sample acquisition, one or more containers configured to receive the swab(s) for storage, instructions for sample provision and setup of a user account, elements configured to associate the sample(s) with the subject (e.g., barcode identifiers, tags, etc.), and a receptacle that allows the sample(s) from the individual to be delivered to a sample processing operation (e.g., by a mail delivery system). In another example, wherein samples are extracted from the user with the help of another entity, one or more samples can be collected in a clinical or research setting from a subject (e.g., during a clinical appointment).

In some embodiments, the compositions in the kit can be provided in a watertight or gas tight container which in some embodiments is substantially free of other components of the kit. For example, the kit components can be supplied in more than one container, e.g., the kit can be supplied in a container having sufficient reagent for a predetermined number of samples, e.g., 1, 2, 3, or greater. One or more components as described herein can be provided in any form, e.g., liquid, dried or lyophilized form. In some embodiments, the components described herein are substantially pure and/or sterile. When the components described herein are provided in a liquid solution, the liquid solution preferably is an aqueous solution, with a sterile aqueous solution being preferred.

The informational material can be descriptive, instructional, marketing or other material that relates to the methods described herein. The informational material of the kits is not limited in form. In some embodiments, the informational material can include information about production of the reagents, concentration, date of expiration, batch or production site information. In some embodiments, the informational material relates to methods for using or administering the components of the kit.

The kit can be provided with various elements included in one package, e.g., a fiber-based, e.g., a cardboard, or polymeric, e.g., a Styrofoam box. The enclosure can be configured to maintain a temperature differential between the interior and the exterior, e.g., the enclosure can provide insulating properties to keep the reagents at a preselected temperature for a preselected time.

In some embodiments, a kit includes a sheet of material configured to be applied to a toilet seat such that when a user sits on the toilet seat, the sheet of material forms a receptacle underneath an anus of the user; disposable gloves; a sample vial with a cap that forms a seal to the sample vial, wherein the cap comprises an elongated utensil component, wherein when the cap is sealed to the sample vial, the elongated utensil component extends into the sample vial; a specimen pouch suitable to contain the sample vial; and a shipping box suitable to contain the specimen pouch. The sheet of material can be made of any suitable material, e.g., paper, plastic, fabric, or textile. In some embodiments, the sheet of material is biodegradable. The kit can include written instructions on use of the kit and components thereof. The kit can include written instructions for performance of a microbiome assay on the biological sample. In some embodiments, a kit can include an identification label and/or a shipping label.

In some embodiments, the system comprises a sample processing platform 110. In some embodiments the sample processing system 110 comprise a next generation sequencing (NGS) platform. Non-limiting examples of NGS platforms include 454 sequencing, SOLiD sequencing, polony sequencing, Illumina® sequencing, Ion Torrent sequencing, sequencing by hybridization, nanopore sequencing, HelioScope sequencing, single molecule real time sequencing, and RNAP sequencing.

In some embodiments, the sample processing platform 110 is configured to receive the sample container of the sampling kit 100. In some embodiments, the sample processing platform is configured to associate the sample container with the subject, for example, through the use of barcodes and a barcode reader. In some embodiments, the sample processing platform is configured to generate a microbiome feature dataset based upon sequencing the nucleic acid content of a microorganism portion of the sample.

In some embodiments, the system comprises at least one computing device 120. As a non-limiting example, the system can comprise 1, 2, 3, 4, 5, or more computing devices. In some embodiments, a computing device 120 is connected to a display 130. Computing device 120 is any suitable computing device, including a desktop computer, server (including remote servers), mobile device, or other suitable computing device. In some embodiments, the system further comprises a personal electronic device 140, also referred to herein as an output device, including but not limited to a smart phone, tablet, smart watch, or other smart device.

In some embodiments, the computing device 120 communicates with the NGS platform of the sample processing platform 110 and identifies a set of microorganisms represented in the microorganism portion based upon performance of a mapping operation on portions of the microbiome sequence dataset. In some embodiments, the computing device 120 receives microbial sequence data comprising a microbiome feature dataset from an aggregate set of samples from a population of female individuals within about a year of giving birth. In some embodiments, the computing device 120 receives a supplementary dataset informative of at least one characteristic associated with the population of individuals and the subject. In some embodiments, the computing device 120 inputs the received datasets into a machine learning model to generate a dietary regimen that promotes eubiosis and thereby promotes maternal and/or fetal wellbeing. In some embodiments, the computing device 120 transmits the dietary regimen to the subject.

In some embodiments, the system further comprises a network 150 that connects the sample processing platform 110 to the computing device 120. The network 150 can be an internal or external network, such as the world wide web. The network 150 can be connected to personal electronic device 140, various other devices, servers, or network equipment for implementing the technology described herein.

In some embodiments, the system further comprises a server 160 and database 170. The server 160 can function to connect the database with other applications of the system. The computing device 120 and server 160 can be connected by a network 150. The database 170 can function to store data output from the sample processing platform 110. The database 170 can also store data obtained from subject questionnaires, reports, or other input using the computing device 120. In some embodiments, algorithms or machine learning models as described herein and other software are stored in database 170 and run on server 160. Additionally, data processed or produced by said algorithms or programs can be stored in database 170.

In some embodiments, the system executes an application or web interface on the computing device 120 or personal electronic device 140. The application or web interface can be stored in database 170 and executed using the server 160.

In another example, an application executing at a personal electronic device (e.g., smart phone, smart watch, head-mounted smart device) can be configured to provide notifications (e.g., at a display, haptically, in an auditory manner, etc.) regarding therapeutic suggestions generated by the therapy model.

Notifications can additionally or alternatively be provided directly through an entity associated with a subject (e.g., a caretaker, a spouse, a significant other, a healthcare professional, a nutrition supplier, etc.). In some further variations, notifications can additionally or alternatively be provided to an entity (e.g., healthcare professional or nutrition supplier) associated with the subject, wherein the entity can administer the therapeutic measure (e.g., by way of prescription, by way of conducting a therapeutic session, by way of food or meal delivery, etc.). Notifications can, however, be provided for therapy administration to the subject in any other suitable manner.

The technology as described herein can be implemented, for example, with any type of hardware and/or software, and can be a pre-programmed general purpose computing device. For example, the system can be implemented using a server, a personal computer, a portable computer, a thin client, or any suitable device or devices. The technology as described herein and/or components thereof can be a single device at a single location, or multiple devices at a single, or multiple, locations that are connected together using any appropriate communication protocols over any communication medium such as electric cable, fiber optic cable, or in a wireless manner. The computing system can include clients and servers. A client and server are generally remote from each other and typically interact through a communication network. The relationship of client and server arises by virtue of computer programs running on the respective computers and having a client-server relationship to each other. In some embodiments, a server transmits data (e.g., an HTML, page) to a client device (e.g., for purposes of displaying data to and receiving user input from a user interacting with the client device). Data generated at the client device (e.g., a result of the user interaction) can be received from the client device at the server.

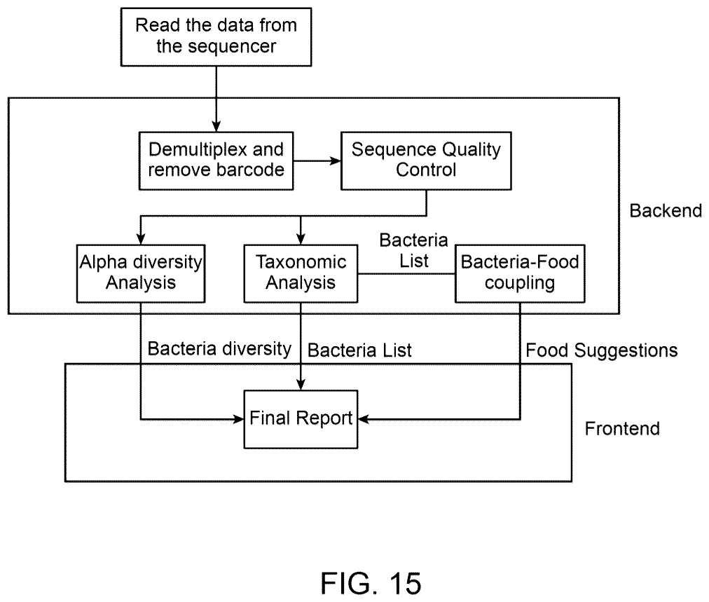

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer to-peer networks). FIG. 15 is an example computer system described herein. The back end component includes the demultiplexing and removal of barcodes, sequence quality control, diversity analysis, taxonomic analysis, bacteria-food coupling. The corresponding front end component includes a final report of bacteria diversity, bacteria list, and food suggestions.

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., CDs, disks, or other storage devices).

The operations described in this specification can be implemented as operations performed by a data processing apparatus on data stored on one or more computer-readable storage devices or received from other sources.

A data processing apparatus can be any kind of apparatus, device, and machine for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of these. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages. The computer program can be deployed in any form, including as a standalone program or as a module, component, subroutine, object, or other unit suitable for use in a computing environment. A computer program can, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA or an ASIC as noted above.

Processors suitable for the execution of a computer program include, for example, both general and special purpose microprocessors, and any one or more processors of any kind of digital computer. Generally, a processor can receive instructions and data from a read only memory or a random access memory or both. The essential elements of a computer are a processor for performing actions in accordance with instructions and one or more memory devices for storing instructions and data. Generally, a computer can also include, or be operatively coupled to receive data from, or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of nonvolatile or non-transitory memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

In some embodiments of any of the aspects, the reference can be a level of the measured parameter in a population of subjects who do not have or are not diagnosed as having, and/or do not exhibit signs or symptoms of dysbiosis. In some embodiments of any of the aspects, the reference can also be a level of, e.g., one or more target microbes in a control sample, a pooled sample from control individuals, or a numeric value or range of values based on the same. In some embodiments, the reference can be the level of one or more target microbes in a sample obtained from the same subject at an earlier point in time, e.g., the methods described herein can be used to determine whether a subject's sensitivity or response to a given therapy or dietary regimen is changing over time.

In some embodiments, the reference level can be the level in a sample of similar sample type, sample processing, and/or obtained from a subject of similar age, sex, and other demographic parameters as the sample/subject for which the microbiome feature dataset is to be determined. In some embodiments, the test sample and control reference sample are of the same type, that is, obtained from the same biological source, and comprising the same composition, e.g., the same number and type of cells.

Treatment can, for example, reverse, alleviate, ameliorate, inhibit, slow down, or stop the progression or severity of a condition associated with a disease or disorder, e.g., a dysbiosis. Treatment can, for example, reduce or alleviate at least one adverse effect or symptom of a condition, disease, or disorder associated with a dysbiosis. Treatment can, for example, reduce or halt a condition. Treatment can, for example, slow progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration, or palliation of the disease state, remission (whether partial or total), and/or decreased mortality, whether detectable or undetectable. Treatment can, for example, relieve a symptom or side-effect of the disease (including palliative treatment).

Further described herein is a system for promoting maternal and/or fetal wellbeing by providing services, nutritional advice, and meal plans to pregnant women based on microbiome. A subject's microbiome profile can be determined by assaying a biological sample of the subject to provide a microbiome profile of the subject. The microbiome profile can be analyzed by a machine learning and/or artificial intelligence (AI) system described herein to determine recommended nutritional regimen or personalized nutritional therapy to improve the health status of the subject by modulating the microbiome of the subject. Nutritional therapies can include subscription-based meal plans or an ingredient list that corresponds to a recommended nutritional regimen.

The subject can adhere to the personalized nutritional therapy for a time period to improve the subject's health status. In some embodiments, a time period is at least a week, at least two weeks, at least three weeks, at least 25 days, at least 30 days, at least a month, at least 2 months, at least 3 months, at least 4 months, at least 5 months, at least 6 months, at least 7 months, at least 8 months, at least 9 months, at least a year, or more.

In some embodiments, a subject is provided or administered a dietary regimen. Providing the dietary regimen can include, for example, communicating the dietary regimen to the subject being treated and/or a medical profession or other individual associated with the subject. To administer a dietary regimen can involve, for example, supplying the subject with foods, probiotics, prebiotics, etc. In some embodiments, administration comprises physical human activity, e.g., an injection or an act of ingestion. Such activity can be performed, e.g., by a medical professional and/or the subject being treated. In some embodiments, administering or providing a dietary regimen is not therapeutic, but rather promotes wellbeing. Alternatively, such administration or provision of advice or a dietary regimen can promote eubiosis, promote a healthy microbiome, promote gut barrier function or integrity, and/or promote healthy gut-related immune function.

### EXAMPLES

### Example 1. Services for Pregnant Women's Nutritional Advice and Meal Plans

Described herein are systems, methods, and kits that provide services, nutritional advice, and meal plans to pregnant women by analyzing their gut bacteria. Raw feature microbiome data derived from pregnant women and/or their infant's biological samples and non-pregnant women can be used to produce models that can be used to improve pregnant women's and/or infants' health.

### Methods

Methods described herein focus on limiting or treating pregnant women's inflammation and discomfort, and reducing the risk of developing illness due to a weakened immune system, all of which can impact the infant's health. Up to 80% of babies born with immune system deficits are related to poor maternal nutrition during pregnancy. The complex composition of the gut microbiome can have a significant impact on the health of the individual. Each person has a different gut microbiome composition, which in turn means each person has different nutritional requirements. A mother's gut microbiome can change drastically throughout pregnancy; therefore, personalized nutrition throughout pregnancy is a benefit.

Metagenomic sequencing technology can be used to impact the health of pregnant women and babies. The technology described herein can provide nutrition advice and meal plans by using NGS technology and machine learning based on the gut microbiome composition to optimize an expecting mother's health and the infant's health.

Genomic sequencing technology can be used to characterize the gut microbiome composition. This information can be processed via algorithms developed by applying machine learning to reference and test data sets to create customized meal plans for expecting mothers to optimize self and baby's health. Subject gut microbiome can be characterized and a customized meal plan can be generated by: (1) providing a test kit to a subject; (2) providing a sample and returning the test kit (e.g., via the post); (3) analyzing the sample on an NGS sequencer; and (4) providing nutritional recommendations and meal plans (e.g., by posting to the mother's personal device via an application).

The technology described herein can exhibit at least the following advantages. Pregnant mothers are empowered by the nutritional recommendation information. The nutritional regimens determined by the methods described herein can help women reduce inflammation. The nutritional regimens determined by the methods described herein can also help reduce the onset of asthma in children and the likelihood of the children developing allergies. The nutritional regimens can also help balance the microbiome of C-section babies and improve overall health.

Ultimately, the complex composition of the gut microbiome can have a large impact on the health of the individual. Each person has different gut microbiome compositions, which in turn means each person has have different nutritional requirements.

Systems and methods described herein can help pregnant and breastfeeding women by providing highly personalized nutritional advice and meal plans. Genomic sequencing technology can be used to characterize the gut microbiome composition. This information can then be used to create customized meal plans for expecting mothers to optimize self and baby's health.

In this example, tailored nutritional advice is generated by: (1) collecting biological samples, such as fecal, saliva, skin, or vaginal microbial samples; (2) extracting DNA, and optionally RNA and/or protein, from the human biological samples; (3) running metagenomics and metatranscriptomics sequencing, such as by using the ILLUMINA Platform or the THERMO FISHER LIFE SCIENCE Platform; and (4) analyzing the next-generation sequencing output by the sequencing platform, such as by using GENEIOUS Software.

### Systems

Provided herein are systems that use a backend server and a database, which can be used to run a MVP (Minimum Viable Product) app, a web console, and/or a website.

The backend server can act as a bridge between the web console and the database. The backend server can be developed on LOOPBACK and an IBM open-source framework. The code can be stored on GITHUB account, in a private repository.

The database can contain data used in the MVP app and web console. Data can be stored on FIRESTORE in a project in SPARK accounts.

The application can connect directly to the backend server and downloads data from the FIREBASE database (SPARK account). In some embodiments, the application can be developed using ANDROID STUDIO and JAVA. In some embodiments, the application can be developed using GLIDEAPP PROTOTYPE and FIGMA.

Images and all static assets used in the MVP app and web console can be stored in Cloud Storage as a project in SPARK accounts.

The web console can be an admin panel that can be used to monitor shipment status, test kit arrival, and manage the data yield from the microbiome sequencing process. The web console can be developed on REACT and REACT-ADMIN. The code can be stored on GITHUB account, in a private repository.

The website can be available on the world wide web at mamamia-eia2019.web.app. The website can be built on ANGULAR and is hosted on FIREBASE HOSTING in a project on SPARK accounts.

The systems described herein can be used as: (1) an analysis tool of raw data from the NGS sequencer; (2) a recipe suggestion application; and/or (3) an in-house algorithm for both pregnant mothers and their infants.

### Example 2. A System and a Method for Pregnant Women's Nutritional Advice and Meal Plans by Analyzing the Microbiome Composition

Nutritional advice and meal plans for pregnant women can be generated through analysis of a subject's microbiome. A kit can be provided to a subject that contains: (a) a sample container containing lysis buffer component; (b) a sample preservation component; and (c) informational material relating to sample site collection for the individual. A prepaid label can also be attached to the shipping box for the individual to ship the kit back to the laboratory. In the laboratory, a metagenomic and metatranscriptomic sequence dataset can be generated based upon the microbiome sample that the individual provided.

Raw feature microbiome data derived from pregnant women and/or infants' biological samples are provided to produce nutritional recommendation models. Machine learning models are trained on individuals' microbiomes at a genus and/or species level, or trained on specific nucleic acids, to predict the originating species, the microbiome community resulting in optimal health for the individual, or other quantitative phenotypes, such as those based on an anthropometric dataset. Simultaneously, an analysis is generated based upon a set of features related to the individual such as those provided by a comprehensive questionnaire describing the individual's lifestyle, anthropometrics, preferences, and health history. The analytical information derived from the analysis can be correlated to the microbiome. Customized nutritional recommendations and meal plans can then be generated and transmitted to a subject's personal device.

### Methods

The machine learning model described herein backends and processes large amounts of data, particularly metagenomics and metatranscriptomics. The machine learning model functions on an omics level across pregnant women's own host data to run analysis simultaneously and provide a recommendation for the nutritional meal plan.

The systems and methods described herein empower pregnant mothers by: (1) reducing inflammation; (2) reducing the onset of asthma in children; (3) reducing the chance of children developing allergies; and (4) balancing out the effect of a healthy microbiome for Caesarean section babies.

Methods described herein can be used to provide personalized nutritional advice and meal plans to pregnant or breastfeeding women to improve health and promote eubiosis. High throughput genomic sequencing technology can be used to characterize the gut microbiome composition, and then this information can be used to create customized meal plans for expecting mothers to optimize self and baby's health.

Described herein are systems and methods for individuals, particularly for pregnant women and corresponding infants, to provide nutritional advice and meal plans by analyzing microbiome composition. In a first model, a sample kit was provided to the individuals in a location remote from the laboratory, the kit including a sample container containing lysis buffer component and a sample preservation component and informational material about sample site collection for the individual. Biological samples such as fecal, saliva, skin, and/or vaginal samples from pregnant women and corresponding infants and/or other individuals were collected and shipped back to the laboratory. In the laboratory, the cells were lysed with magnetic beads. Nucleic acids such as DNA and RNA were extracted from the collection samples. DNA and RNA were both processed in different ways.

For DNA samples, the processing method included: (1) end repair and purify pooled amplicons; (2) ligate and nick-repair; and (3) purify the adapter-ligated and nick-repaired DNA. For RNA samples, the processing method included: (1) digestion (e.g., with a DNase); (2) RT-PCR; (3) cDNA synthesis; (4) ligate and nick-repair; and (5) purify the adapter-ligated and nick-repaired cDNA. After the nucleic acid was purified, amplicons were prepared, and adapters were ligated to the nucleotides. Nucleic acids were then associated with the microbiome using nucleic acid barcodes. Libraries were produced by adding to the DNA adapter molecules, each comprising a universal priming sequence, binding site, and barcode sequences. The DNA libraries were made and prepared to run with a high throughput sequencer (e.g., ILLUMINA, LIFE SCIENCE, or NANOPORE TECHNOLOGY platforms).

A process for collecting and sequencing genomic data includes: (a) collecting biological samples such as fecal, saliva, skin, and/or vaginal samples from pregnant women and their infants and/or other individuals; (b) adding solutions to preserve the nucleotides; (c) lysing the cells with beads; (d) extracting nucleic acids such as DNA and RNA from collection samples, comprising (i) for DNA: (1) performing end repair and purifying pooled amplicons; (2) ligating and performing nick-repair; and (3) purifying the adapter-ligated and nick-repaired DNA; or (ii) for RNA: (1) performing a digestion; (2) performing RT-PCR; (3) performing cDNA synthesis; (4) ligating and performing nick-repair; and (5) purifying the adapter-ligated and nick-repaired DNA; (e) preparing amplicons; (f) purifying the amplification products (e.g., in a PCR plate or tubes); (g) ligating adaptors; (h) barcoding the nucleic acids; (i) calculating DNA input for library preparation; (j) running the library on a high throughput sequencer (e.g., Illumina, Life Science or Nanopore technology platforms); and (k) analyzing the output data from the sequencer using the machine learning model described herein.

Herein, machine learning models were trained on individual microbiomes at a genus and/or species level, or trained using other groups of nucleic acids to predict the originating species, optimal growth temperatures, or other quantitative phenotypes such as those based on an anthropometric dataset. The in-house algorithm was trained to clean the data. As a non-limiting example, bacterial diversity data (e.g., Alpha-Beta diversity Cluster) was input into scripts in PYTHON and BASH to design nutrition recommendations or meal plans. The framework QUIIME2 was used to perform tasks such as distributing macronutrients across all the meals. The provided meals were designed using each of the four macronutrients: carbohydrates, fiber, protein, and fat. The thresholds were based on the proportion of each macronutrient and daily recommended allowance. Trained multilayer perceptrons and linear regressions were used to design the recipe and meal plan. As a non-limiting example, meals were selected from recipes using methods such as PYTHON, Scikit-Learn Library, and Deep Learning Library. To manage the calories of meals, the following methods were used: PYTHON, Machine Learning Library, Deep Learning Library, and Reinforcement Learning Library. The ingredients clustering and recipe suggestions were written in PYTHON, using a K-NN classifier from a Machine Learning Library. In some embodiments, an analysis was generated based upon a set of features related to the individual such as those provided by a comprehensive questionnaire describing the individual's lifestyle, anthropometrics, preferences, and health history. The analytical information derived from the analysis was translated to the microbiome, and then customized nutritional recommendations and meal plans were output to the mother's personal device via an application.

The libraries were analyzed using methods including but not limited to: Machine Learning; Deep Learning; Reinforcement Learning; Bacterial Diversity. In some embodiments, nucleic acids were prepared for analysis, e.g., using polymerase chain reaction (PCR). In some embodiments, modifications to the library included incorporating an adaptor.

### Systems

The promotion of eubiosis in a subject is undertaken with a system that includes a backend server and a database, which can be used to run an MVP app, a web console, and/or a website.

The backend server acts as a bridge between the web console and the database. The backend server was developed on LOOPBACK and an IBM open-source framework. The code is stored on GITHUB account, in a private repository.

The database contains data used in the MVP app and web console. Data is stored on FIRESTORE in a project in SPARK accounts.

The application connects directly to the backend server and downloads data from the FIREBASE database (SPARK account). In some embodiments, the application can be developed using ANDROID STUDIO and JAVA. In some embodiments, the MVP application can be developed using GLIDEAPP PROTOTYPE and FIGMA.

Images and all static assets used in the MVP app and web console are stored in Cloud Storage as a project in SPARK accounts.

The web console is an admin panel that can used to check on shipment status, test kit arrival, and manage the data yield from the microbiome sequencing process. The web console was developed on REACT and REACT-ADMIN. The code is stored on GITHUB account, in a private repository.

The website is built on ANGULAR and is hosted on FIREBASE HOSTING in a project on SPARK accounts.

The systems described herein can be used as: (1) an in-house algorithm to provide individuals with a personalized nutrition book; (2) a device (e.g., chip) to monitor the response after a meal for individuals who take the meal plans; and (3) a tool to support pharmaceuticals for personalized microbiome probiotic recommendations.

### Example 3. Clinical Trial for Analyzing Microbiome Composition from Pregnant Women

A clinical trial can be performed to analyze microbiome samples from pregnant women or expectant mothers collected at various stages of the pregnancy. The study can include 60 healthy expectant mothers divided into two groups as summarized in TABLE 1. Meals consumed by the expectant mothers in the study can be recorded for the first 14-30 days. Using microbiome sequencing techniques and other methods described herein, the microbial composition of the samples can be characterized to associate microbial composition with nutrient requirements to optimize the microbiome eubiosis. The nutrient requirements can be based on the nutrient intake of the expectant mothers as well as the microbial composition of the samples.

This protocol can be extended to samples collected from pregnant women having gestational diabetes, preeclampsia, asthma, allergies, or another condition. These samples are sequenced, and meals are recorded for the first 14-30 days. Microbial compositions of the healthy pregnant women can be compared with the microbial compositions of the pregnant women having gestational diabetes, preeclampsia, asthma, allergies, or other condition.

TABLE 1

| | |
|---|---|
| Group 1 | Collect 10 gut microbiome samples in the first trimester |
| | (e.g., once notified of pregnancy status) |
| | Collect 10 gut microbiome samples in the second trimester |
| | Collect 10 gut microbiome samples in the third trimester |
| Group 2 | Collect 10 gut microbiome samples in the first trimester |
| | (e.g., once notified of pregnancy status) |
| | Collect 10 gut microbiome samples in the second trimester |
| | Collect 10 gut microbiome samples in the third trimester |

Patients can provide microbiome samples to a clinic, e.g., an OB-GYN clinic. For example, a physician can recommend an expectant mother to have a gut microbiome analysis. The expectant mother can provide consent and a stool sample to the clinic during an office visit.

Alternatively, study participants can ship samples directly to a study facility. For example, an expectant mother can provide a stool sample using an at-home gut microbiome kit. The kit can include materials for preserving the stool sample to ensure viability during transport and processing. For instance, the microbiome kit can include a test tube containing a DNA shield buffer, which maintains viability of the stool sample for up to 30 days at room temperature. Example test tubes include DNA/RNA shield fecal collection tubes, which can be designed for the collection and preservation of nucleic acids from stool specimens. These stool collection tubes can preserve the microbial composition of a sample, while inactivating contaminating viruses. Samples stored in these collection tubes can be stable at ambient temperature, and can be frozen for longer-term storage. A spoonful of the fecal specimen can be placed into stool collection tubes that are optionally pre-filled with a preservative. After sample collection, the collection tube can be shaken vigorously to ensure proper mixing and stabilization. The kit can include a box or package to contain the test tube for convenient transport. The kit can also include a shipping label to expedite the shipping process. In some cases, the kit can be picked up by a representative of the study facility, for example, twice weekly.

After samples are processed and analyzed, patients can access a virtual portal via a personal device to input daily meal and nutrients intake, thereby providing a report of nutrition intake. Patients can also view personalized dietary recommendations determined by the systems described herein. In some cases, patients can view revised dietary recommendations in response to the report of nutritional intake.

### Example 4: Clinical Trial for Analysis of Dietary Influence on Microbiome During Pregnancy and Gestational Diabetes Risk

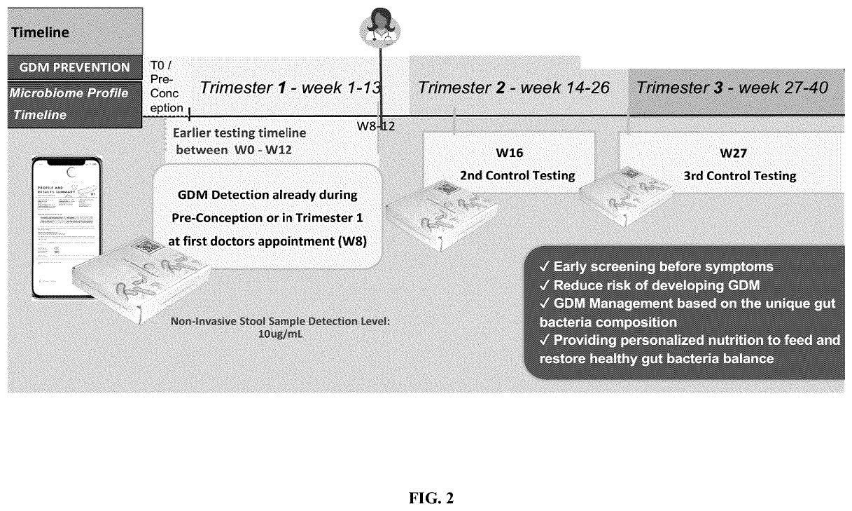

FIG. 2 illustrates a timeline for Gestational Diabetes Mellitus (GDM) screening using microbiome screening kits of the disclosure. GDM screening using the systems and methods described herein can be performed as early as 0-12 weeks of gestation, or even during pre-conception. The screening test is non-invasive stool sample detection test. In contrast, current GDM screenings are typically performed after 24-28 weeks of gestation. These GDM screenings are performed by glucose tolerance testing, which requires fasting, ingestion of a glucose solution, and blood draw.

A clinical trial can be performed to analyze stool, vaginal, and oral samples of pregnant women diagnosed with gestational diabetes (GDM) and healthy women to identify changes in the microbiome following a 28-day dietary modification that was created using an AI-based platform. A first set of stool, vaginal, and oral samples taken before the start of the 28-day diet can be used establish a baseline of a participant's microbiome, and a second sample can be taken following the conclusion of the diet to identify any changes.

Sample site justification: The samples collected from participants can be oral, fecal, and vaginal. The oral and vaginal can be collected via swabs at the OB/GYN office. The fecal can be collected via a take-home collection kit by the participant. Two fecal samples can be collected, one before starting the 28-day trial, and one after the completion of the trial. The oral and fecal samples can be used to monitor and analyze the gut microbiome. Although both sample sites are part of the digestive system, the oral microbiome can constitute a very different composition of taxa than the intestinal microbiome. Oral and fecal samples can indicate gut microbiome state with respect to pregnancy and pregnancy complications. The vaginal microbiome can also be important to the assessment. While the vaginal microbiome may not be directly associated with diet and food consumption, changes in the vaginal microbiome can indicate absorption of nutrients and the blood glucose levels. The vaginal microbiome is also closely linked to the growing fetus and changes to the reproductive system during pregnancy.

Personnel and roles in the study:
1. Clinic personnel for recruitment—Blinded to study and results
   a. Identify participants, provide information on the study, and sign consent forms
   b. Transmit the forms, personal information (name, address, medical records) to study coordinators and other personnel
2. Study coordinators—Not blinded
   a. Receive forms from the clinic, assign IDs, remove participant identifiers, save information on database, create participant profiles for diet/exercise reporting application, confirm grocery orders to patients
   b. Transmit all necessary de-identified information to the study personnel
   c. Supervise data transfer between personnel, ensure quality control, and coordinate tasks
   d. Answer participant questions on mobile instant messaging portal
   e. Communicate with the participants for follow-ups
3. Principal investigators—Blinded
   a. Supervise and conduct the study protocols
   b. Collect the data and results
   c. Analyze the results
4. Data entry interns—Blinded
   a. Transfer data from meal input to database
   b. Transfer medical record data to database

Participants can be divided equally into a control group and an intervention group. The control group can be formed of healthy pregnant women with no diagnosed complications, while the intervention group can be formed of women diagnosed with GDM. Both groups can be further divided equally into random diet groups and recommended diet groups as summarized in TABLE 2.

Recruitment and group assignment: Since the study includes more than 1 group, a system of randomization can be implemented to eliminate any bias. The participants do not choose a group. The categorization procedure can be as follows:
1. The non-pregnant group that includes women who agree to participate. A physician confirms the women are not pregnant and healthy (i.e., no pregnancy-related condition detected).
2. The pregnant group that includes women who agree to participate. A physician confirms the women are pregnant.
3. Categorization of a participant into healthy or unhealthy groups through the physicians' assessment of the health of the participant.
4. Categorization of a participant into a randomized diet or recommended diet through blind drawing of numbers from a set.

Participants of the random diet group would be instructed to follow a own regular diet with no meal restrictions or recommended food items or meal plans. These participants would provide a detailed record of foods consumed as the randomized (control) diet. On the other hand, the recommended diet group would be instructed to follow a provided meal plan.

The recommended diet group can be provided with the same food ingredients to cook in whichever manner each participant chooses. The target population can be chosen based on the following criteria: (a) Pregnant women between the ages of 18-45; (b) The GDM groups are diagnosed during their current pregnancy; (c) The recommended diet groups do not include women with food allergies; and (d) The recommended diet GDM group do not include women prescribed with insulin.

TABLE 2

| | Healthy | GDM | |
|---|---|---|---|
| | Participants | Participants | Total |
| Random Diet | 4-5 | 4-5 | 8-10 |
| Recommended Diet | 4-5 | 4-5 | 8-10 |
| Total | 8-10 | 8-10 | 16-20 |

### Step 1.

To extract necessary information regarding previous or current illness and/or symptoms that can help improve the accuracy of the study, a complete medical history can be obtained for each participant. Conditions such as allergies, gastrointestinal diseases, chronic illnesses, miscarriages, obstetric and gynecological conditions and procedures can affect the integrity and composition of the gut microbiome. Any medication or procedures prescribed, and conditions or complications of pregnancy that develop can also be used to correlate with the nutritional data and microbiome composition changes. These medical records are not shared with third parties, and if used in publications do not include any participant identification information.

Disease development and blood glucose levels can be monitored during the course of pregnancy. Random 1-hour and 2-hour postprandial, HbA1c, and fasting glucose levels can be obtained. To establish a baseline of each participant's microbiome, the participants can provide stool, oral, and vaginal samples, which can be collected within 3 days of starting the protocol. Sample mislabeling, sample contamination, failure to provide medical history, or failure to provide the sample within 3 days of protocol initiation can result in disqualification of the participant.

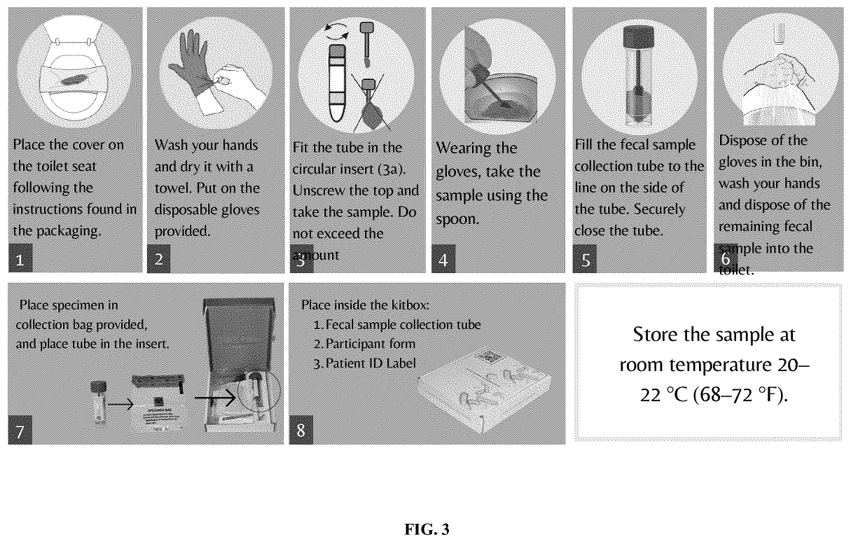

Testing kits for sample collection can be equipped to stabilize samples at point of collection, facilitate simultaneous detection of nucleotides from bacteria and viruses from a single sample, and extend storage complexity to 24 weeks of ambient temperature stability for nucleotide transportation, storage costs, and complexity. The kits can allow identification of living or metabolically active bacteria at the time of sample collection through RNA expression profiling and possess format compatibility with high-throughput processing increases efficiency and minimizes sample handling errors. Patients can be provided with instructions for sample collection, including the example instruction card depicted in FIG. 3.

### Step 2.

Each participant can commit to a 28-day regimen of diet. Both random diet groups can be asked to follow their health care providers' recommendations for nutrition and medication and log their diets on a daily basis to an online portal. A detailed food preference list can be filled out before the diets are provided to ensure that the women do not receive any disliked foods, condiments, or spices. The recommended diet groups can be provided with ingredients for breakfast, lunch, and two snacks, and a freedom of choice for dinner daily. Four total deliveries can be made over the 28-day duration of the study. Participants can receive the items planned for consumption weekly in each delivery. Certain items can be delivered either once or twice over the duration of the study. Food items delivered to the recommended diet group and the duration of consumption can be as follows: (i) chia seeds, delivered only once in the first week delivery, meant to last for the full 28 days; (ii) microwave popcorn, delivered twice, once in the first and once in the third deliveries, meant to last 2 weeks each; and (iii) oatmeal, delivered twice, once in the first and once in the third deliveries, meant to last 2 weeks each.

Each participant can be required to log meals daily and upload pictures of the items consumed and not consumed from the meals. Using these pictures, and through regular communication from a study coordinator, study administrations can identify what was consumed and calculate nutritional values for each meal.

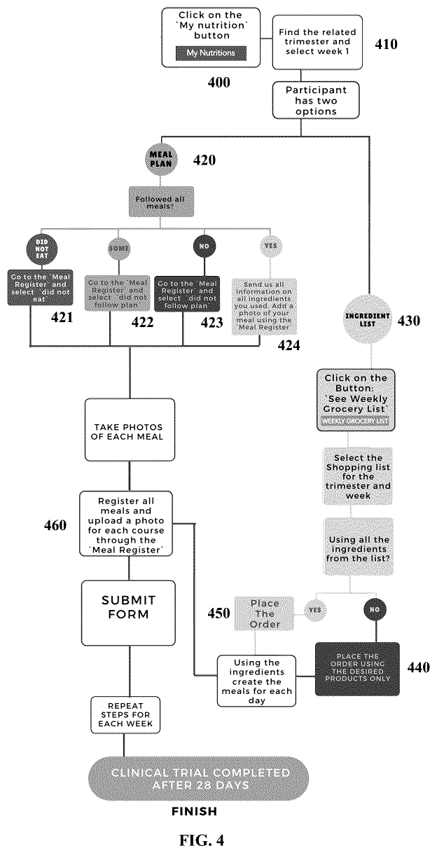

A flow diagram for an example user-facing web interface for submission of participant meal data and ordering of meals is provided in FIG. 4. Through this interface, participants can report compliance with a meal plan and indicate whether food items consumed or not consumed by the participant. A participant can access the portal of the application by selecting the “My Nutritions” button 400. The participant can then select her related trimester and the study week 410. After specifying the trimester and study week, the participant can select the diet option: a meal plan 420 or an ingredient list 430.

Through the meal plan option 420, the participant can report on whether the meal plan was followed. In the reporting interface 420, the participant can specify consumption of an unspecified meal 421, report partial compliance to a meal plan 422, report noncompliance with a meal plan 423, or report complete compliance 424. Example responses include: did not eat (DID NOT EAT; 421), ate some (SOME; 422), did not follow plan (NO; 423), and followed plan (YES; 424). Response can be entered into the “Meal Register” portal. In each case, the participant can then proceed to upload images of meals before and after consumption by the participant 460. These steps can be repeated weekly by the participant for a total of 4 weeks (28 days).

Alternatively, a participant can select the ingredient list option 430 to view a weekly grocery list (“See Weekly Grocery List”). Based on the grocery list, the participant can select ingredients of interest (NO) or all ingredients from the grocery list (YES). After selection of ingredients, the participant can be directed to an online shopping portal. Through this portal, the participant can directly place an order for the selected ingredients of interest 440 or place an order for all the ingredients 450. Once received, the participant can then use the ingredients to prepare daily meals. As with the meal plan option, the participant can report all meals and upload photos through on the Meal Register portal 460. These steps can be repeated weekly by the participant for a total of 4 weeks (28 days).

All participants can be required to provide a second set of stool, oral, and vaginal samples upon conclusion of the 28-day period. Failure to log meals or upload pictures for 3 successive days, or a total of 6 or more days during the 28-day period results in disqualification of the participant. Failure to provide the second set of samples within 3 days of the trial conclusion results in disqualification of the participant from only this step.

Participants can be monitored for glycemic excursions, e.g., via phone follow-up by study investigators with discussion of blood glucose monitoring and careful documentation of progress through pregnancy. Immediate referral to a practitioner for treatment occurs if 20% of glucose values by four times a day self-glucose monitoring exceeds the target thresholds (FBG>95 mg/dL; 1 hr>140 mg/dL or 2 hr>120 mg/dL). After 1 week of therapy, if women in the intervention group demonstrate a 20% increase in glucose levels, medical management is instituted and the subject is withdrawn from the study.

### Step 3.

Genomic sequencing of approximately 2,500 base pair amplicons that span the 16S and 23S rRNA genes (StrainID™) and mapping to a 16S-ITS-23S long read database (SBanalyzer™ and Athena™) can be used to identify organisms at the strain level in each sample, infer Amplicon Sequence Variants using DADA2 (ASVs), and identify established correlations between certain species and specific nutrients and diseases. High resolution tracking of bacteria in the mother's microbiome community can provide a fingerprint distinct to the mother's microbiome at stages of pregnancy relative to overall health. A fastq file at the strain level can be obtained for each sample. Failure to acquire the file due to contamination or failure to generate any sequence at all can disqualify the results of the sample.

### Step 4.

Statistical analysis can be used to calculate correlation between GDM and the microorganisms in all participants and record the effect of any intervention on the composition of all applicable organisms. Blood glucose levels can also be correlated to the same 28-day period of diet. The analysis can establish a correlation between GDM and at least one microorganism, as well as the diet regiment in participants.

Raw sequencing data from Step 3 can be processed and mapped to bacterial genomes to obtain strain-level information, as well as to characterize bacterial pathways present in each sample. Species-level identification can be performed using a modified Ensemble-based software. Strain-level identification can be also performed using our machine learning algorithm. Bacterial gene and pathway information can be annotated using Humann2. PICRUSt can be used to predict bacterial functions based on the operational taxonomic units (OTU) table generated from the rRNA gene sequencing data. The accuracy of PICRUSt predicted data can be estimated by comparing output against the metagenomic-sequence data on the subset of samples.

The resulting metagenomic data can be further analyzed using the HUMAnN (HMP unified metabolic analysis network) pipeline to allow for annotation and testing of metabolic pathway abundance as an alternative to isolated bacterial genes. Enriched or depleted microbial strains associated with GDM and dietary intervention groups can be identified using Fisher's exact test. All of the samples from participants can be processed by using a metagenomic method to yield raw microbiome data features including a maternal microbiome database and microbial taxonomy. The maternal microbiome database can be represented by mathematical functions in terms of food, prebiotic, probiotics, supplements, and medical/disease conditions.

Groups of raw microbiome data features can then be aggregated into custom microbiome labels to capture the healthy and unhealthy characteristics. Samples from participants can be processed by using a metagenomic method to yield raw microbiome data features including a maternal microbiome database and microbial taxonomy. Microbiota pattern and abundance can be characterized at a genus/species/strain level OTUs. Statistical presence, absence, and abundance can be calculated by unweighted UniFrac distances between the groups, as determined by 16S-ITS-23S rRNA gene sequences.

Computational methods can be performed by a QIIME2 pipeline that operates primarily through the use of QIIME Artifacts (.qza files), which serve as “filing cabinets” for microbiome data and metadata (descriptive data of the core data including type, format, and how the data were generated). Alignment of the trimmed, merged and length filtered read set prepared in the previous step (length 150-260 base pairs nucleotides) can ensure each contig comprises only closely related sequences. A curated database can be generated that is specific to Consensus Sequences list containing the unused reads, representing the non-clustered unique sequences. The pipeline can be built with the UNIX command-line environment, bash commands, data formatting using regular expressions, and basic scripting in the Unix shell with a series of examples and exercises via a remote server. The pipeline can function as bioinformatics software for analysis of microbial population data, downstream population genome assembly, alignment/mapping, SNP genotyping, PCoA, population structure analysis, and outlier tests.

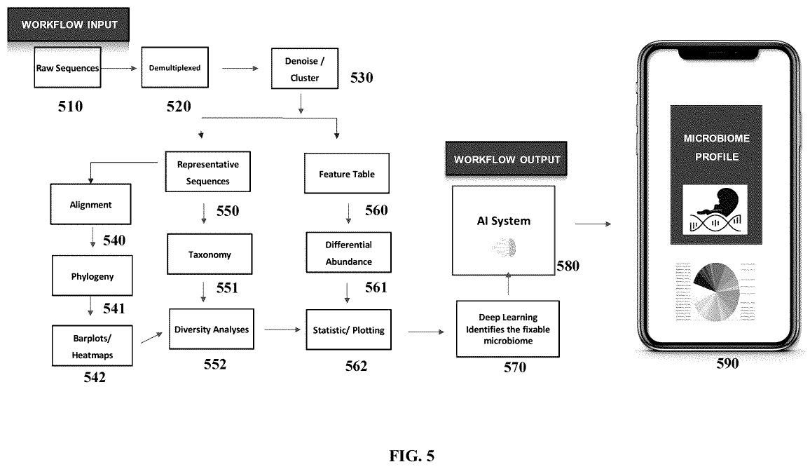

An example workflow for processing genomic data is provided in FIG. 5. In this workflow, raw sequences 510 can be demultiplexed 520. Demultiplexed sequences can then be denoised and clustered 530 to generate representative sequences 550 and a feature table 560. The representative sequences 550 can then undergo sequence alignment 540 prior to phylogenic analysis 541. Phylogenic analysis 541 can be used to generated barplots and/or heatmaps 542 that characterize the microbiome composition based on the sequencing data. The representative sequences 550 can also be used to assign taxonomy 551. The barplots and/or heatmaps 542 and taxonomy 551 can each undergo diversity analyses 552. The feature table 560 generated from denoising/clustering 530 can be applied to determine relative or differential abundance 561. Diversity analyses 552 and differential abundance analysis 561 can then be statistically analyzed and plotted 562. A deep learning algorithm 570 can be used to process the statistical data to identify a fixable microbiome composition. The microbiome data can then be processed by the AI system 580. The microbiome data can also be transmitted to a personal device 590, such as a personal device of a subject.

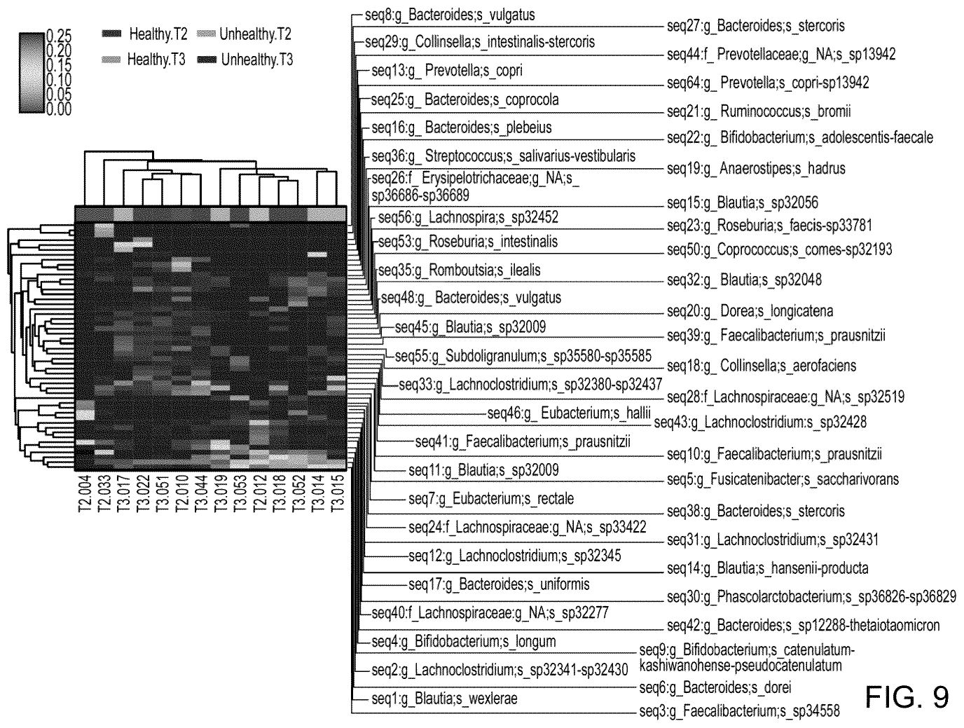

FIG. 9 is an example microbiome heatmap based on data generated by the methods disclosed herein. The heatmap was generated using unique amplicon sequences inferred from raw reads using the Dada2 pipeline sequences. Taxonomy assignment was performed using Uclust from Qiime package. Taxonomy was assigned with a 16S database that is internally designed and curated using methods disclosed herein.

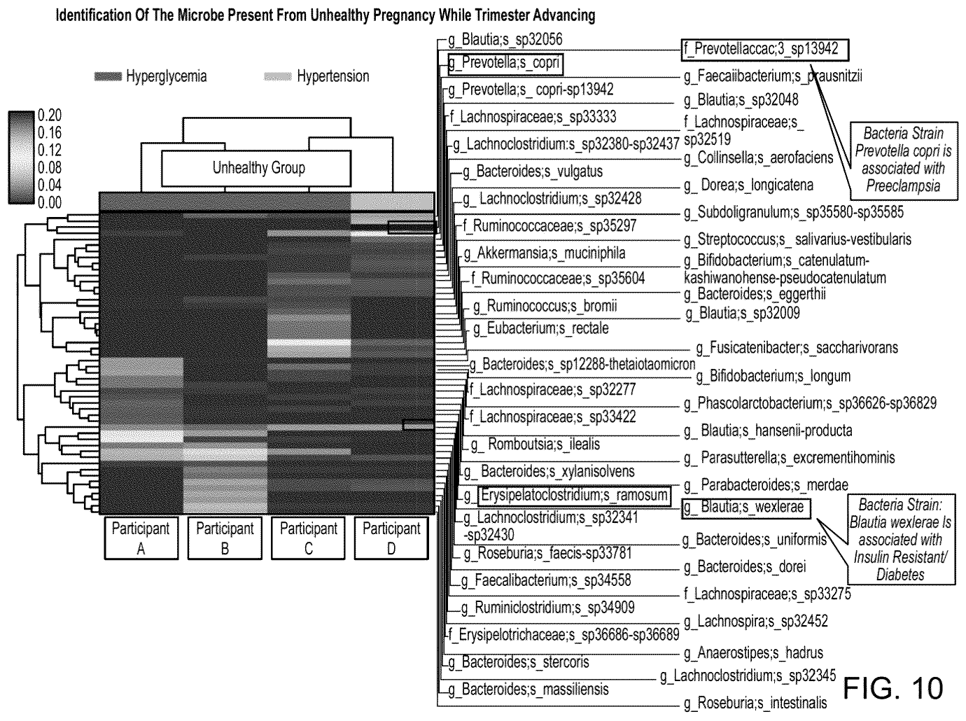

FIG. 10 is another example microbiome heatmap based on data generated by the methods disclosed herein. Both heatmaps were assembled using the same bioinformatics analysis method. FIG. 9 is based on raw sequencing data from healthy and unhealthy participants within Trimester 2 and Trimester 3. FIG. 10 is based on raw sequencing data from unhealthy participants within the cohort, specifically focusing on hypertension and hyperglycemia.

Healthy pregnant women: Healthy can be determined by the OB/GYN and can be defined as participants who are considered primarily free of the unhealthy conditions described herein. Women with any known food allergies can be excluded from this study. These pre-existing conditions can be routinely determined by the OB/GYN for any patients under his/her care and would be disclosed by the patients to the OB/GYN. The healthy group can include pregnant women between the ages of 18-45, no known food allergies, not diagnosed with any condition that deems participation impossible by an OB/GYN.

Unhealthy pregnant women (disease target groups of GDM): Eligible participants for the study are women aged 18 years to 35 years, pregnant at 20 weeks of gestation with a history of GDM and/or a prepregnancy BMI of ≥30 kg/m 2. Women who satisfy the dietary criteria for GDM according to international guidelines (fasting plasma glucose≥92 mg/dL and/or 1 h post-test glycemia≥180 mg/dL and/or 2 h post-test glycemia≥153 mg/dL) can be enrolled.

Exclusion criteria were type 1 or type 2 diabetes, or GDM diagnosed before 20 weeks of gestation; use of medication that influences glucose metabolism, such as continuous therapy with oral corticosteroids or metformin; multiple pregnancy; physical disability; current substance abuse; severe psychiatric disorder; and significant difficulty in cooperating
Trimester 0: Preconception
Trimester I: the first trimester is from week 1 to the end of week 12.
Trimester II: the second trimester is from week 13 to the end of week 26.
Trimester III: The third trimester is from week 27 to the end of the pregnancy.
Trimester IV: After birth; Postnatal/Breastfeeding

Women with any pathological conditions before or during pregnancy known diabetes mellitus, hypertension, cardiovascular, pulmonary, autoimmune, joint, liver or kidney diseases, thyroid dysfunction, cancer, any other disease/condition), no compliance to the study protocol, on prebiotics/probiotics, antibiotics or any drug during pregnancy can be excluded.

Unhealthy can be determined by the collaborating OB/GYN and can be defined as participants who have been determined to have one or more of the following pre-existing conditions: hypertension, diabetes (type I or type 2). gestational diabetes, a history of smoking, severe psychiatric disorders, substance abuse Unhealthy group is determined to be pregnant women diagnosed with GDM during their current pregnancy, no other diseases or food allergies can be in the inclusion criteria. Women prescribed with insulin can be excluded.

No healthy vs unhealthy groups are defined. However, participants discuss collecting data on food consumed over the continuous 28-day period, and data on medical history and any conditions that develop during participation including gastrointestinal diseases, preeclampsia, gestational diabetes, allergies and associated health profiles and risks from each participant

Patients with GDM can show a predominance of Firmicutes (increasing during the gestational period) and Bacteroidetes (whose relative abundance decreased), while the infant gut microbiota can be characterized by an increased relative abundance of Actinobacteria and Proteobacteria. Hierarchical clustering analysis at genus level can show a separation between the infant microbiota and the mother microbiota. Infants can display a higher abundance of Bifidobacterium, Streptococcus, Escherichia, Staphylococcus, and Enterococcaceae, while mothers can have a more complex microbiota composition.

### Example 5: Application of Artificial Intelligence for Analysis of Microbiome and Nutritional Influence on High-Risk Pregnancies

Two sets of gut, vaginal, and oral metagenomic samples can be collected from a total of 200 healthy and high-risk pregnant participants on day 1 and day 28 of a 28-day period. TABLE 3 shows timeframe, samples, and information collected from each participant. Data pertaining to food consumed over the continuous 28-day period can also be collected, and data on medical history and any conditions that develop during participation including gastrointestinal diseases, preeclampsia, gestational diabetes, and allergies. Each set can count as an entry in the database. Samples deemed to be compromised due to packaging damage, container damage, mixed labels, or missing labels can be discarded.

TABLE 3

| Entry | | | | |
|---|---|---|---|---|
| No. | Time | Sample Set | Nutrition | Conditions |
| 1 | Day 1 of | Gut, vaginal, | Diet before | Medical history |
| | participation | stool samples | 28-day | |
| | | collected | period | |
| 2 | Day 28 of | Gut, vaginal, | Diet during | Any medical |
| | participation | stool samples | 28-day | conditions during |
| | | collected | period | 28-day period |

A StrainID® Kit from Shoreline Biome can be used to amplify the long read NGS amplicon (2,500 bases spanning the bacterial 16S-ITS-23S rRNA genes), and map sequences to long read Athena database using DADA2 to infer amplicon sequence variants (ASVs). Based on the unique combination of ASVs, closely related enteric bacteria in the human population can be identified and differentiated. A PacBio Sequel II® system can be used for long read high-throughput sequencing. Alternatively, an Oxford Nanopore Technologies (ONT) MiniIon® long read sequencer in combination with a Shoreline Biome NanoID® reagent kit and companion SBanalyzer software can be employed.

### Bioinformatics Pre-Processing

Fastq raw sequence files can undergo a pre-processing phase of demultiplexing to determine where each sample read originates. To reduce sequence errors and dereplication of sequences, the demultiplexed reads can undergo denoising to generate features in the form of amplicon sequence variants (ASVs) or operational taxonomic units (OTUs). The resulting features and representative sequences can be used for downstream analysis, such as taxonomic classification to identify bacteria species in the different samples. Resulting taxonomic data are combined with a database of food, prebiotic, probiotics, supplements, and medical/disease conditions to create an integrated database. The features and representative sequences in the database can then be aggregated into custom microbiomes that are labeled to capture healthy and unhealthy characteristics. The healthy microbiome, or balanced microbiome profile, can be an aggregated assessment of overall diversity of the OTU and ratios of active beneficial and harmful microbes. Statistical presence, absence, and abundance can be calculated by unweighted UniFrac distances between the groups as determined by 16S-ITS-23S rRNA gene sequences.

### Database Enrichment

Natural Language Processing can be used to process and extract relevant nutrient information from published and peer-reviewed articles where different microbial species in present in the taxonomic data have been implicated as associated with disease states and the maternal gut microbiome. By using different text preprocessing pathways, different pre-trained BERT Extractive Summarization models, and different summary length specifications, a set of 6 different summaries that capture different elements of each microbiome can be produced. This plan can result in a dashboard that displays key terms which are disease related. Food/diet and maternal health outcomes can also be presented in the summary for quick review. Raw text extracted from the building system can also be reviewed from the same interface.

### Identification of Bacterial Strain Biomarkers

Species-level identification of bacteria can be performed using an ensemble of methods for maternal bacterial species identification. Strain-level identification can be performed using machine learning algorithms. Finally, bacterial gene and pathway information can be annotated using Humann2. PICRUSt can be used to predict bacterial functions based on the operational taxonomic units (OTU) table generated from the 16S-ITS-partial 23S rRNA gene sequencing data. Accuracy of PICRUSt predicted data can be estimated by comparing the data against the metagenomic-sequence data on the subset of samples. The resulting metagenomic data can be further analyzed using the HUMAnN (HMP unified metabolic analysis network) pipeline to allow for annotation and testing of metabolic pathway abundance as an alternative to isolated bacterial genes.

Enriched or depleted microbial strains associated with maternal GDM and dietary intervention groups can be identified using Fisher's exact test. For mother-baby pairs, at 37 weeks of pregnancy and 90 days, respectively, bacterial strains and/or related metabolites transmitted from the healthy pregnant women to vulnerable pregnant women can be identified and used as direct targets for intervention.

### Recommendation Systems and Artificial Intelligence

A recommendation system (RS) can utilize information from a personalized healthy target microbiome to suggest the path towards achieving the target in the host and ensuring the health benefits. For example, a knowledge-based RS, where recommendations are made using a limited number of approved dietary prescriptions, can be used. Microbiome-aware diet recommendations can also be generated from content-based or collaborative filtering.

### Online Portal

An online portal is provided that serves as a gateway by which users and medical professionals can access results. In the portal, patients can review results in concert with corresponding recommendations for the diet. Medical professionals can also review the results of patients, the recommendations to treatment plans, order further samples, or contact the patients directly.

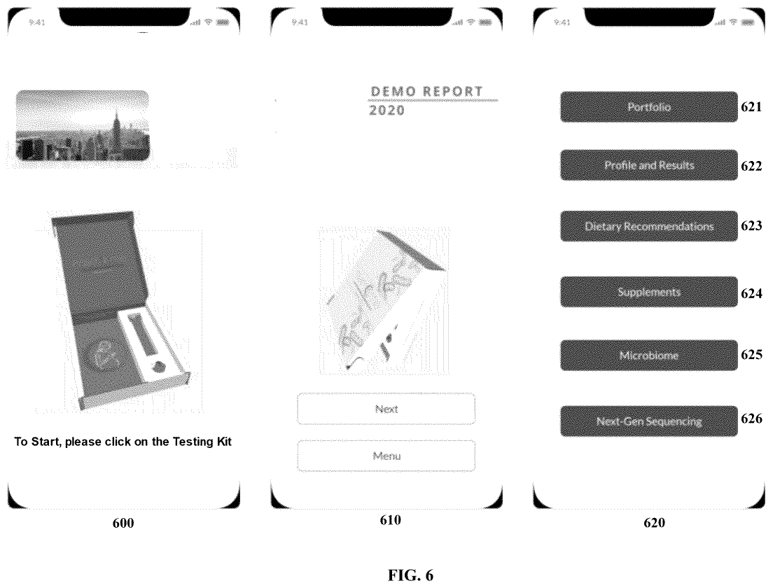

An example smartphone interface for accessing the portal is provided in FIG. 6. In the smartphone interface, after tapping on the landing page 600, a user can view a report through report interface 610 or access a menu interface 620. In the menu interface, the user can view the user's portfolio 621, which can include background health and demographic information. The user can also view the user's profile and test results 622, suggested dietary recommendations 623, suggested supplement intake 624, microbiome profile 625, and Next-Gen sequencing data 626.

### Data

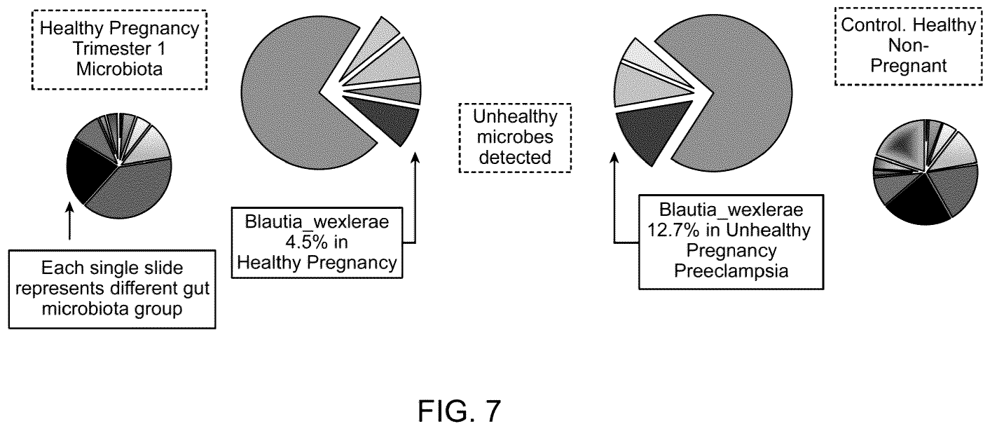

An example microbiome report is provided in FIG. 7. In the microbiome report, participants and practitioners can review microbiome data in the form of the relative proportion of microbial taxa identified in participant samples. Participant data can also be compared to healthy control subjects. Throughout pregnancy, the microbiome composition can change.

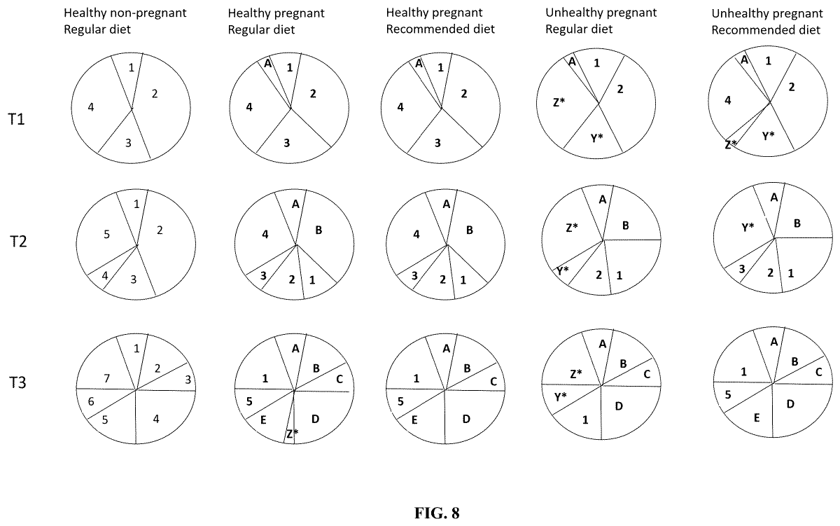

FIG. 8 is a chart illustrating example data that is obtained from participants in the first trimester (T1), second trimester (T2), and third trimester (T3) of pregnancy. The leftmost column depicts baseline proportions of gut microbial taxa (1-7) in healthy non-pregnant women following a regular diet. As time progresses and seasons change throughout T1, T2, T3, bacterial taxa may also change.

The second column depicts proportions of microbial taxa in pregnant individuals following a regular diet. Gut microbial taxa and proportions can change in healthy pregnant women on a regular diet as time (i.e., trimester) progresses. Pregnancy-associated bacteria, pregbiota, appear (A, B, C, D, E), while some baseline bacteria disappear. In this example, taxa Z* is a bacterial taxon found in unhealthy pregnant women.

The third column depicts proportions of microbial taxa in healthy pregnant individuals following a recommended diet generated by the methods disclosed herein. Healthy pregnant women receiving a recommended diet exhibit gut microbial taxa and proportions that are similar to healthy pregnant women on a regular diet. The healthy pregbiota do not always substantially change with the recommended diet. Unhealthy pregbiota such as Z* can be eliminated.

The fourth column depicts proportions of microbial taxa in unhealthy pregnant individuals following a normal diet. Unhealthy pregnant women following a regular diet show different bacterial taxa and compositions vs. healthy pregnant women. In this example, unhealthy pregbiota (Z*, Y*) are present.

The fifth column depicts proportions of microbial taxa in unhealthy pregnant individuals following a recommended diet generated by the methods disclosed herein. Unhealthy pregnant women receiving a recommended diet slowly show changes in microbial taxa and composition reflecting healthy pregbiota present in healthy pregnant women on the recommended diet. In this example, unhealthy pregbiota X* and Y* are eventually eliminated through the recommended diet.

### Example 6: An Example GDM Diet Plan Generated by Systems Described Herein

Gestational diabetes mellitus (GDM) is a growing public health concern. Maternal obesity and poor dietary intake could be implicated as causes of GDM. Dietary polyphenols and fiber can mitigate the risk of diabetes and its complications, but little is known about the efficacy of these foods in preventing GDM.

Diet plans established using systems and methods described herein can be based on maternal nutrition intake and microbiome composition. Early pregnancy intervention can be critical for favorable modification of maternal weight gain and glycemia and can promote sustained compliance. Diets established using systems and methods described herein can be tailored to the nutritional needs of a pregnant woman according to the trimester. The meals can be categorized into a variety of the meal kits including twice of the home grocery delivery services. The GDM meal plans can be prescribed to mothers who are at the risk of developing with GDM during pregnancy. In diagnosed patients, GDM meal plans along with insulin therapy can be prescribed. The mother can be also tasked with regular self-testing of blood glucose levels several times a day to avoid hypoglycemia or hyperglycemia. The approach to GDM diet plans is to limit carbohydrate levels in a similar way to Type II diabetes patients, while increasing proteins and fatty acids to allow the growing fetus to receive all the necessary requirements to continue developing until birth. In this trial, two groups receive provided GDM plans according to their trimester. One group can be healthy pregnant women as controls, and the other group as be pregnant women diagnosed with GDM. The meal plan as be designed, revised, and approved by expert nutritionists and physicians.

Effects of low-carbohydrates, whole blueberry, grape and soluble fiber on primary outcomes of maternal microbiome profiles in women at high-risk of developing GDM can be examined.

### Key Point 1: Carbohydrates

Increased consumption of carbohydrates that resist digestion by the host, such as dietary fiber, has been associated with a reduced risk of obesity, type 2 diabetes, certain gastrointestinal disorders, and coronary heart disease. More recently, the ability of indigestible carbohydrates to influence the gut microbiome has become of considerable interest for human health. Some important functions of carbohydrates include guiding the gut microbiome, and through this mechanism, protecting the host from pathogens, educating the immune system, regulating metabolism, and fueling nerve growth and repair. Understanding the interactions between structures of food carbohydrates, intestinal functions and health can be critical to establishing an optimal diet plan to reverse GDM.

### Key Point 2: Fiber

Fiber, and a wide array of dietary bioactive compounds including polyphenols, from two different sources of food can have completely different monosaccharide compositions, bond linkages, degree of polymerization (number of single monosaccharides linked to one another), and in turn, biological functions. Polyphenolic flavonoids that are largely present in functional foods, such as dark-colored berries, grapes, tea, olives, and whole grains, have been associated with decreased risks of diabetes in observational studies.

### Key Point 3: Polyphenol-Rich Blueberries and Grapes

METHODS: Intervention (280 g whole blueberries and 12 g soluble fiber per day) and standard prenatal care (control). Both groups can receive nutrition education and maintain 24-h food recalls throughout the study.

An AI-based maternal health management platform is used to provide a grocery, meal plan, and wellness/lifestyle program for pregnancy and breastfeeding to treat or reduce the likelihood of GDM.

Substitution—Asian Diet

Deliveries—Twice a week service, upon on request service, or a grocery subscription plan (e.g., a 9-month subscription plan)

Medical Background—The medical history, genetic predispositions, and demographic information of participants can be considered in the analysis of the microbiome and nutritional intake.

This GDM diet plan is established based on the relationship between the microbiome and nutrition intake. The GDM meal plans can be prescribed to mothers who are at the risk of developing with GDM during their pregnancy. The GDM meal plans can also be prescribed to mothers already diagnosed with GDM along with insulin therapy. The prescribed GDM diet can be designed to limit carbohydrate levels, while increasing proteins and fatty acid similar to diets recommended for Type II diabetes patients. Such a diet can allow a growing fetus to receive necessary nutrients to continue developing until birth. This microbiome-based diet can reduce the likelihood of maternal weight gain, hyperglycemia, and postpartum diabetes, while promoting sustained compliance. The meals can be categorized into a variety of the meal kits, including, for example, twice weekly home grocery delivery services. The meal plan can be designed, revised, and approved by expert nutritionists and physicians. Example diet plans are provided in TABLE 4 and TABLE 5. TABLE 6 shows an example grocery list.

TABLE 4

| | breakfast | am snack | lunch | pm snack |
|---|---|---|---|---|
| Sun. | Banana Pancakes | 1 cup Sliced | Blackened Salmon | Cherry Chocolate |
| | topped with 1 tbsp | Cucumbers + 1.5 oz | Tacos | Chia Pudding |
| | maple syrup + ½ | Low-Fat Cheese | (3 oz blackened | (Make ahead of |
| | cup blueberries | | salmon, 2 corn | time- Mix all |
| | (Mash 2 ripe | | tortillas or 1 whole | ingredients together |
| | bananas, mix with | | wheat tortilla, top | and place in |
| | 2 eggs, ½ cup | | with pineapple slaw) | refrigerator 1 hour |
| | whole wheat flour, | | Pineapple Slaw (1 | before consuming. |
| | and 2 tsp baking | | cup shredded | Mix again before |
| | powder. Add a | | cabbage, ½ cup | eating. 2 tbsp chia |
| | drizzle of oil to the | | pineapple diced, ¼ | seeds, 1 tsp maple |
| | skillet and cook on | | cup red bell pepper | syrup, ½ cup low-fat |
| | medium heat until | | finely chopped, ¼ | milk, 1 cup frozen |
| | cooked through. | | cup red onion finely | cherries, 1 oz |
| | Makes 4 servings. | | chopped, and juice of | chopped or chips of |
| | Save leftovers for | | one lime) | dark chocolate) |
| | Tuesday and | | | |
| | Thursday, and | | | |
| | freeze one serving) | | | |
| Mon. | Southwest | 1 cup Sliced Peppers + | Pina Colada Greek | 1 Banana + 1 tbsp |
| | Scramble Tacos | ¼ cup Guacamole | Yogurt Parfait | Peanut Butter for |
| | (Scramble 2 eggs | | (1 cup Greek | dipping |
| | with ¼ cup salsa | | yogurt- choose low | |
| | and 1 cup kale. | | sugar coconut if you | |
| | Place scramble | | prefer; 1 cup diced | |
| | into 2 corn tortillas | | pineapple; 2 tbsp | |
| | or 1 whole wheat | | shredded | |
| | tortilla) | | unsweetened | |
| | | | coconut) | |
| Tues. | Banana Pancakes | 1 cup Sliced | Fresh Mediterranean- | Cherry Chocolate |
| | topped with 1 tbsp | Cucumbers + ¼ cup | style Pasta Salad | Chia Pudding |
| | maple syrup + ½ | Hummus | (1 cup cooked whole | (Make ahead of |
| | cup blueberries | | wheat pasta, 3 oz | time- Mix all |
| | | | chicken, 1 cup baby | ingredients together |
| | | | kale ½ cup diced | and place in |
| | | | cucumbers, ½ cup | refrigerator 1 hour |
| | | | diced grape tomato, | before consuming. |
| | | | 1.5 oz diced | Mix again before |
| | | | mozzarella cheese, ¼ | eating. 2 tbsp chia, 1 |
| | | | cup diced canned or | tsp maple syrup, ½ |
| | | | fresh artichokes, add | cup LF milk, 1 cup |
| | | | homemade | frozen cherries, 1 oz |
| | | | vinaigrette) | chopped or chips of |
| | | | | dark chocolate) |
| Wed. | Avocado + Egg | 1 Banana + 1 tbsp | Pina Colada Greek | 1 cup Sliced |
| | Toast + ½ cup | Peanut Butter for | Yogurt Parfait | Cucumbers or Bell |
| | Raspberries | dipping | (1 cup Greek | Peppers + ¼ cup |
| | (Toast one slice of | | yogurt- choose low- | Hummus |
| | whole wheat | | sugar coconut if you | |
| | bread, mash ¼ | | prefer; 1 cup diced | |
| | avocado and top | | pineapple; 2 tbsp | |
| | on toast, cook one | | shredded | |
| | egg to your | | unsweetened | |
| | preference and top | | coconut) | |
| | on avocado toast) | | | |
| Thurs. | Banana Pancakes | 1 cup Sliced Peppers + | Grilled veggie and | 1 Grapefruit + 1 oz |
| | topped with 1 tbsp | ¼ cup Guacamole | hummus wrap | Walnuts |
| | maple syrup + ½ | | (Top 1 whole wheat | |
| | cup blueberries | | tortilla with ¼ cup | |
| | | | hummus, and grilled | |
| | | | veggies- zucchini, | |
| | | | carrots, peppers, red | |
| | | | onion) | |
| Fri. | Southwest | Cherry Chocolate | Fresh Mediterranean- | 1 cup Sliced Peppers + |
| | Scramble Tacos | Chia Pudding | style Pasta Salad | ¼ cup Guacamole |
| | (Scramble 2 eggs | (Make ahead of | (1 cup cooked whole | |
| | with ¼ cup salsa | time- Mix all | wheat pasta, 3 oz | |
| | and 1 cup kale. | ingredients together | chicken, 1 cup baby | |
| | Place scramble | and place in | kale ½ cup diced | |
| | into 2 corn tortillas | refrigerator 1 hour | cucumbers, ½ cup | |
| | or 1 whole wheat | before consuming. | diced grape tomato, | |
| | tortilla) | Mix again before | 1.5 oz diced | |
| | | eating. 2 tbsp chia, 1 | mozzarella cheese, ¼ | |
| | | tsp maple syrup, ½ | cup diced canned or | |
| | | cup LF milk, 1 cup | fresh artichokes, add | |
| | | frozen cherries, 1 oz | homemade | |
| | | chopped or chips of | vinaigrette) | |
| | | dark chocolate) | | |
| Sat. | Avocado + Egg | 1 Grapefruit + 1 oz | Grilled Veggie and | 1 cup Sliced |
| | Toast + ½ cup | Walnuts | Hummus Wrap | Cucumbers + 1.5 oz |
| | Raspberries | | (Top 1 whole wheat | Low-Fat Cheese |
| | (Toast one slice of | | tortilla with ¼ cup | |
| | whole wheat | | hummus, and grilled | |
| | bread, mash ¼ | | veggies: zucchini, | |
| | avocado and top | | carrots, peppers, red | |
| | on toast, cook one | | onion) | |
| | egg to your | | | |
| | preference and top | | | |
| | on avocado toast) | | | |

TABLE 5

| Weekday | Breakfast | Snack 1 | Lunch | Snack 2 | Dinner | Snack 3 |
|---|---|---|---|---|---|---|
| Saturday | Whole | 4 T | Tuna Salad | ½ C Plain | Tofu & | 1 oz Dark |
| | Wheat Pita, | Hummus, | | Greek | Veggies w/ | Chocolate |
| | ½ | ½ C | | Yogurt, | Black Bean | |
| | Avocado, 1 | Carrots, | | ½ C | Sauce | |
| | egg, 2 oz | ½ C | | Blueberries, | | |
| | Cheddar | Cucumber | | 1 T Chia | | |
| | Cheese | | | Seeds | | |
| Sunday | Omelet - 1 | Almond | Tofu & | 1 small | Spicy Kung | ½ C |
| | Egg, 1 C | Flour Bar | Veggies w/ | apple, 2 T | Pao | Blueberries |
| | Veggies, 2 T | Spiced | Black Bean | unsalted | Chicken | |
| | Cheddar | Carrot Cake | Sauce | peanut | | |
| | Cheese, ½ | | | butter | | |
| | Avocado | | | | | |
| Monday | Blueberry | Whole | Spicy Kung | 4 T | Beef & | Almond |
| | Almond | Wheat Pita, | Pao | Hummus, | Broccoli | Flour Bar |
| | Chia | 2 oz | Chicken | ½ C | | Spiced |
| | Pudding | Cheddar | | Carrots, | | Carrot Cake |
| | | Cheese | | ½ C | | |
| | | | | Cucumber | | |
| Tuesday | Whole | 4 T | Beef & | ½ C Plain | Thai Style | 1 oz Dark |
| | Wheat Pita, | Hummus, | Broccoli | Greek | Noodles | Chocolate |
| | ½ | ½ C | | Yogurt, | | |
| | Avocado, 1 | Carrots, | | ½ C | | |
| | egg, 2 oz | ½ C | | Blueberries, | | |
| | Cheddar | Cucumber | | 1 T Chia | | |
| | Cheese | | | Seeds | | |
| Wednesday | Omelet - 1 | Almond | Thai Style | 1 small | Cashew | ½ C |
| | Egg, 1 C | Flour Bar | Noodles | apple, 2 T | Chicken | Blueberries |
| | Veggies, 2 T | Spiced | | unsalted | | |
| | Cheddar | Carrot Cake | | peanut | | |
| | Cheese, ½ | | | butter | | |
| | Avocado | | | | | |
| Thursday | Blueberry | Whole | Cashew | 4 T | Chicken | Almond |
| | Almond | Wheat Pita, | Chicken | Hummus, | Teriyaki | Flour Bar |
| | Chia | 2 oz | | ½ C | | Spiced |
| | Pudding | Cheddar | | Carrots, | | Carrot Cake |
| | | Cheese | | ½ C | | |
| | | | | Cucumber | | |
| Friday | Pork & | 4 T | Chicken | ½ C Plain | Beef & | ½ C |
| | Chive | Hummus, | Teriyaki | Greek | Veggies w/ | Blueberries |
| | Dumplings | ½ C | | Yogurt, | Black Bean | |
| | | Carrots, | | ½ C | Sauce | |
| | | ½ C | | Blueberries, | | |
| | | Cucumber | | 1 T Chia | | |
| | | | | Seeds | | |

TABLE 6

Produce:
4 bananas
1.5 cups blueberries
1 cup raspberries
2.5 cups diced pineapple
3 cups cherries (can be frozen)
1 cup shredded cabbage
2 cucumbers
3 bell peppers
1 avocado
2 grapefruits
Grape tomatoes
Baby spinach
Baby kale
Carrots
Zucchini
Red onion
Guacamole (pre-made or make your own)
Meat/Protein/Dairy:
8 eggs
2 cup Greek yogurt (can choose coconut flavored if you'd like)
6 ounces of chicken
3 ounces of salmon
Hummus
Low-fat cheese (e.g., mozzarella cheese)
Peanut butter
Walnuts
Shredded unsweetened coconut flakes
Other:
Chia seeds
Maple syrup
Dark chocolate/dark chocolate chips
Salsa
Canned artichokes
Grains:
Whole wheat flour
Whole wheat bread
8 corn tortillas OR 4 whole wheat tortillas

### Example 7: An Example Computational Microbiome Analysis Pipeline

The computational microbiome analysis methods described herein utilizes bioinformatics and predictive modeling as tools for clinical diagnostics, early screening, disease prevention, and personalized medicine. These methods can be used to extract a large amount of information about the genome, transcriptome, proteome, metabolome, and phenome of patients. The information available from these omics data is rich enough to allow screening and early detection of multiple diseases as well as the detection of therapeutic targets in drug discovery. The methods can employ sequencing technologies such as NGS, microarrays, RNA-Seq, and/or MALDI-TOF to characterize the microbiome of a patient. Such methods can be used for both clinical and at-home testing.

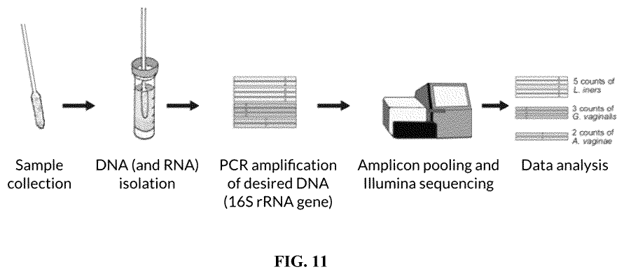

The human microbiome is the collection of approximately 100 trillion microorganisms living in and on the human body. These microorganisms live in and on many sites in our bodies, including the skin, lungs, gastrointestinal tract, mouth, and others. These microorganisms interact with our human cells, playing crucial roles in various cellular and bodily processes. The microbiome can play a role in the development of disease, and microbiome-targeting methods can be promising therapeutic approaches to diseases such as Inflammatory Bowel Disease, Irritable Bowel Syndrome, and bacterial infections. FIG. 11 is an example workflow for characterizing the microbiome, which are described in detailed below.

### Step 1: Sample Collection

Microbiome samples (for example, saliva, cheek/tongue swab, skin, and/or fecal samples) are first collected from patients according to the region of interest of the study, and then stored using protocols to avoid contamination and maintain temperature stability to prevent the DNA and RNA from degrading.

### Step 2: DNA (and RNA) Isolation

Chemical DNA and RNA extraction methods are then used to extract the DNA and RNA of interest to the study, the analysis of which can yield a profile of the bacterial composition of the microbiome.

### Step 3: PCR Amplification of Desired DNA and Sequencing

The Polymerase Chain Reaction DNA amplification method, in which primers and DNA polymerase make many copies of DNA strands through a thermocycling process, is used to amplify the target DNA. Depending on the study, the desired DNA can either be the whole genome, in which case a shotgun sequencing method can be used (random fragments are sequenced and then assembled into complete sequences by computer), or the desired DNA can be a particular region of interest. For example, an example region of interest can be the 16S rRNA gene, which encodes for a critical ribosomal protein. The 16S rRNA gene is highly evolutionarily conserved and is present in all bacteria. The 16S rRNA gene allows for species identification based on sequence differences. In this case, only the region of interest can be PCR-amplified and sequenced, e.g., by amplicon pooling and sequencing using Illumina® platforms.

### Step 4: Data Analysis

Computational methods are used to process the raw sequencing data and identify bacterial species present in the sample and the relative abundance thereof. Computational processing can be performed using QIIME2.

The QIIME2 python package is a microbiome analysis tool for performing a complete microbiome analysis from initial raw sequencing data through the end result of analysis and visualization. A QIIME2 pipeline operates primarily through the use of QIIME Artifacts (.qza files), which are essentially filing cabinets for microbiome data and metadata (descriptive data of the core data including type, format, and how the data were generated). These QIIME artifacts can be used to store and pass data between steps of a QIIME2 pipeline.

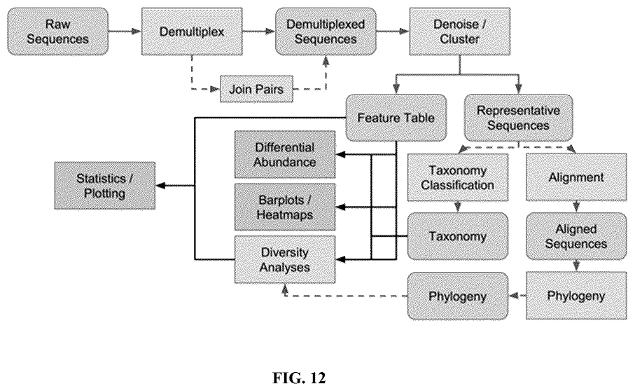

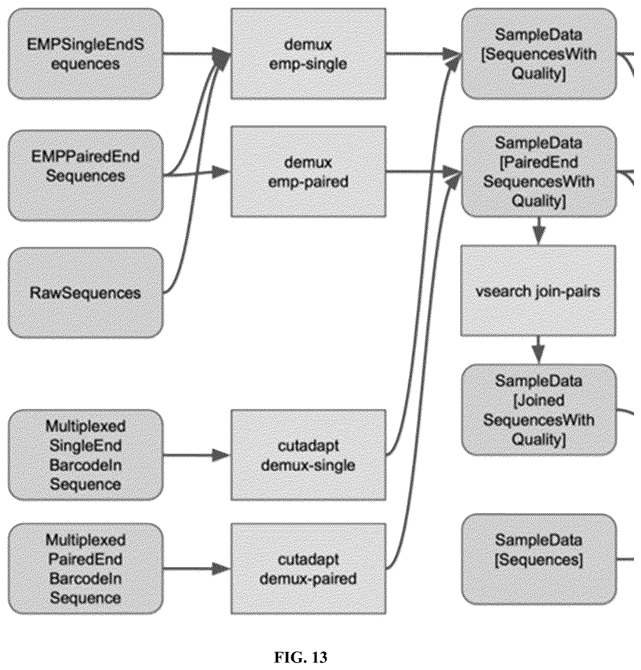

As shown in FIG. 12, raw sequences are demultiplexed to generate demultiplexed sequences. Sequence pairs can also be joined to generate demultiplexed sequences. Demultiplexing is the separation of grouped raw sequencing data by sample origins. A QIIME Package can provide functions for demultiplexing different types of input data as shown in FIG. 13. These input data can include single end sequences (EMPSingleEndSequences), paired end sequences (EMPPaired End Sequences), and raw sequences (RawSequences). Single end sequences can be demultiplexed into demultiplexed single sequences (demuxemp-single). Paired end sequences can be demultiplexed paired sequences (demuxemp-paired). Raw sequences can be demultiplexed into demultiplexed single sequences. Demultiplexed data can be stored in the same QIIME artifact.

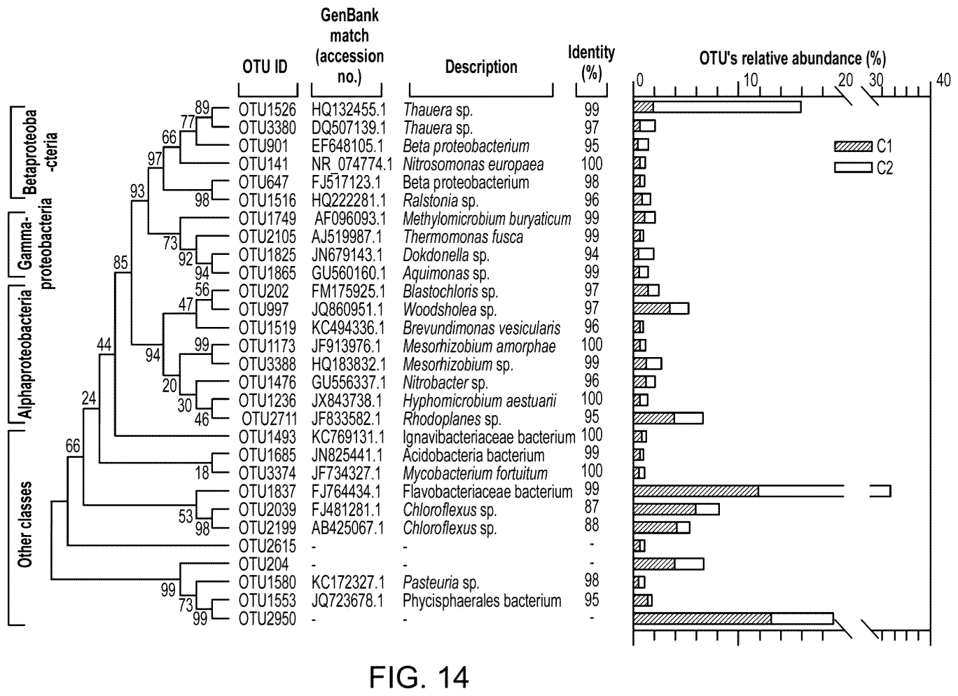

The demultiplexed sequences can then undergo denoising and clustering. Denoising can remove or correct noisy reads present in the data. Dereplication can reduce file sizes and quantitate the frequency of overlapping reads. After denoising and clustering, a feature table and representative sequences can be generated. Accordingly, the feature table can used to generate analytical data, including statistical analyses, plotting, differential abundance of bacterial species, barplots or heatmaps, and diversity analyses. Representative sequences can be used to generate taxonomy classifications and undergo sequence alignment to generate phylogeny analyses. Taxonomy classifications and phylogeny analyses can further contribute to the bacterial diversity analyses. Multiple available clustering methods in QIIME can be used to produce OTUs from reads using reference datasets as shown in FIG. 14.

### EMBODIMENTS

Embodiment A01. A method comprising analyzing a result of a gut microbiome sequencing assay of a biological sample of a subject to provide an outcome, and determining a health status of the subject based on the outcome, wherein the subject is a female within about a year of giving birth.

Embodiment A02. The method of embodiment A01, wherein the health status of the subject characterizes an effect of gut microbiome activity on reproductive processes within about a year of giving birth.

Embodiment A03. The method of embodiment A01, wherein the health status of the subject characterizes an effect of gut microbiome activity on pregnancy.

Embodiment A04. The method of embodiment A01, wherein the health status of the subject characterizes an effect of gut microbiome activity on nursing.

Embodiment A05. The method of embodiment A01, wherein the health status of the subject is risk of dysbiosis.

Embodiment A06. The method of embodiment A01, wherein the health status of the subject is risk of dysbiosis of a gut microbiome of the subject.

Embodiment A07. The method of embodiment A01, wherein the health status of the subject is risk of gestational diabetes.

Embodiment A08. The method of embodiment A01, wherein the health status of the subject is risk of preterm birth.

Embodiment A09. The method of any one of embodiments A01-A08, wherein the biological sample is stool.

Embodiment A10. The method of any one of embodiments A01-A09, further comprising, prior to the analyzing, performing the gut microbiome sequencing assay on the biological sample of the subject.

Embodiment A11. The method of embodiment A10, wherein the performing the gut microbiome sequencing assay comprises next generation sequencing.

Embodiment A12. The method of embodiment A10, wherein the performing the gut microbiome sequencing assay comprises 16S rRNA sequencing.

Embodiment A13. The method of embodiment A10, wherein the performing the gut microbiome sequencing assay comprises 16S-ITS-23S rRNA sequencing.

Embodiment A14. The method of any one of embodiments A01-A13, further

comprising determining a recommended dietary regimen for the subject based on the result of the gut microbiome sequencing assay of the biological sample of the subject.

Embodiment A15. The method of embodiment A14, further comprising sending to the subject a supply of nutrition that corresponds to the recommended dietary regimen.

Embodiment A16. The method of any one of embodiments A01-A15, further comprising receiving from the subject a report of nutritional intake of the subject, and providing to the subject a revised dietary regimen in response to receiving the report of nutritional intake.

Embodiment A17. The method of any one of embodiments A01-A16, wherein the subject is pregnant.

Embodiment A18. The method of any one of embodiments A01-A16, wherein the subject is preconception.

Embodiment A19. The method of any one of embodiments A01-A16, wherein the subject is currently involved in a course of nursing a child of the birth.

Embodiment A20. The method of any one of embodiments A01-A16, wherein the subject is lactating.

Embodiment A21. The method of any one of embodiments A01-A20, wherein the subject is human.

Embodiment B01. A method comprising analyzing a result of a microbiome sequencing assay of a biological sample of a subject to provide an outcome, and determining a recommended dietary regimen for the subject based on the outcome, wherein the subject is a female within about a year of giving birth.

Embodiment B02. The method of embodiment B01, further comprising sending to the subject a supply of nutrition that corresponds to the recommended dietary regimen.

Embodiment B03. The method of any one of embodiments B01-B02, further comprising, prior to the analyzing, performing the microbiome sequencing assay on the biological sample of the subject.

Embodiment B04. The method of any one of embodiments B01-B03, wherein the microbiome sequencing assay comprises next generation sequencing.

Embodiment B05. The method of any one of embodiments B01-B04, wherein the microbiome sequencing assay comprises 16S rRNA sequencing.

Embodiment B06. The method of any one of embodiments B01-B05, wherein the microbiome sequencing assay comprises 16S-ITS-23S rRNA sequencing.

Embodiment B07. The method of any one of embodiments B01-B06, further comprising receiving from the subject a report of nutritional intake of the subject, and providing to the subject a revised dietary regimen in response to receiving the report of nutritional intake.

Embodiment B08. The method of any one of embodiments B01-B07, further comprising determining a health status of the subject based on the outcome.

Embodiment B09. The method of embodiment B08, wherein the health status of the subject characterizes an effect of microbiome activity on reproductive processes within about a year of giving birth.

Embodiment B10. The method of embodiment B08, wherein the health status of the subject characterizes an effect of microbiome activity on pregnancy.

Embodiment B11. The method of embodiment B08, wherein the health status of the subject characterizes an effect of microbiome activity on nursing.

Embodiment B12. The method of embodiment B08, wherein the health status of the subject is risk of dysbiosis.

Embodiment B13. The method of embodiment B08, wherein the health status of the subject is risk of dysbiosis of a gut microbiome of the subject.

Embodiment B14. The method of embodiment B08, wherein the health status of the subject is risk of gestational diabetes.

Embodiment B15. The method of embodiment B08, wherein the health status of the subject is risk of preterm birth.

Embodiment B16. The method of any one of embodiments B01-B15, wherein the biological sample is stool.

Embodiment B17. The method of any one of embodiments B01-B15, wherein the biological sample is saliva.

Embodiment B18. The method of any one of embodiments B01-B15, wherein the biological sample is vaginal fluid.

Embodiment B19. The method of any one of embodiments B01-B15, wherein the biological sample is blood.

Embodiment B20. The method of any one of embodiments B01-B19, wherein the subject is pregnant.

Embodiment B21. The method of any one of embodiments B01-B19, wherein the subject is preconception.

Embodiment B22. The method of any one of embodiments B01-B19, wherein the subject is currently involved in a course of nursing a child of the birth.

Embodiment B23. The method of any one of embodiments B01-B19, wherein the subject is lactating.

Embodiment B24. The method of any one of embodiments B01-B23, wherein the subject is human.

Embodiment C01. A computer program product encoded on a non-transitory computer-readable medium comprising code that instructs performance of a method, the method comprising:
a) providing a computational nutrition system, the computational nutrition system comprising:
1) a data receiving module;
2) a data processing module;
3) a health status module; and
4) a reporting module;
b) receiving by the data receiving module data obtained from a gut microbiome assay of a subject, wherein the subject is a female subject who is within about a year of giving birth;
c) processing by the data processing module the data obtained from the gut microbiome assay of the subject to provide an assay result;
d) analyzing by the health status module the assay result to provide an analysis of a health status of the subject, wherein the health status characterizes an effect of gut microbiome activity on reproductive processes within about a year of giving birth; and
e) reporting to the subject by the reporting module the analysis of the health status of the subject.

Embodiment C02. The computer program product of embodiment C01, wherein the computational nutrition system further comprises:
5) a nutrition recommendation module; and
6) a nutrition reporting module,
   wherein the method further comprises:
f) determining by the nutrition recommendation module a recommended dietary regimen for the subject based on the assay result; and
g) reporting to the subject by the nutrition reporting module the recommended dietary regimen for the subject.

Embodiment C03. The computer program product of embodiment C02, wherein the computational nutrition system further comprises:
7) a nutrition delivery instruction module, wherein the method further comprises:
h) instructing by the nutrition delivery instruction module a nutrition supplier with instructions to deliver to the subject a supply of nutrition that corresponds to the recommended dietary regimen.

Embodiment C04. The computer program product of embodiment C03, wherein the instructing by the nutrition delivery instruction module the nutrition supplier with instructions to deliver to the subject the supply of nutrition that corresponds to the recommended dietary regimen comprises providing to the nutrition supplier a description of foods that correspond to the recommended dietary regimen.

Embodiment C05. The computer program product of embodiment C04, wherein the instructing by the nutrition delivery instruction module the nutrition supplier with instructions to deliver to the subject the supply of nutrition that corresponds to the recommended dietary regimen comprises providing to the nutrition supplier a schedule for delivery to the subject of the foods that correspond to the recommended dietary regimen.

Embodiment C06. The computer program product of embodiment C03, wherein the computational nutrition system further comprises:
8) an update receipt module;
9) a revision module; and
10) a revision reporting module,
   wherein the method further comprises:
i) receiving by the update receipt module an update from the subject, wherein the update describes nutritional intake by the subject;
j) revising by the revision module the recommended dietary regimen for the subject based on the update from the subject to provide a revised dietary regimen; and
k) reporting to the subject by the revision reporting module the revised dietary regimen.

Embodiment C07. The computer program product of any one of embodiments C01-006, wherein the health status characterizes an effect of gut microbiome activity on pregnancy.

Embodiment C08. The computer program product of any one of embodiments C01-006, wherein the health status characterizes an effect of gut microbiome activity on nursing.

Embodiment C09. The computer program product of any one of embodiments C01-008, wherein the subject is pregnant.

Embodiment C10. The computer program product of any one of embodiments C01-008, wherein the subject is preconception.

Embodiment C11. The computer program product of any one of embodiments C01-008, wherein the subject is currently involved in a course of nursing a child of the birth.

Embodiment C12. The computer program product of any one of embodiments C01-008, wherein the subject is lactating.

Embodiment C13. The computer program product of any one of embodiments C01-C12, wherein the subject is human.

Embodiment D01. A computer program product encoded on a non-transitory computer-readable medium comprising code that instructs performance of a method, the method comprising:
a) providing a computational nutrition system, the computational nutrition system comprising:
1) a data receiving module;
2) a data processing module;
3) a nutrition recommendation module; and
4) a nutrition reporting module;
b) receiving by the data receiving module data obtained from a microbiome assay of a subject, wherein the subject is a female subject who is within about a year of giving birth;
c) processing by the data processing module the data obtained from the microbiome assay of the subject to provide an assay result;
d) determining by the nutrition recommendation module a recommended dietary regimen for the subject based on the assay result; and
e) reporting to the subject by the nutrition reporting module the recommended dietary regimen for the subject.

Embodiment D02. The computer program product of embodiment D01, wherein the computational nutrition system further comprises:
5) a nutrition delivery instruction module,
   wherein the method further comprises:
f) instructing by the nutrition delivery instruction module a nutrition supplier with instructions to deliver to the subject a supply of nutrition that corresponds to the recommended dietary regimen.

Embodiment D03. The computer program product of embodiment D02, wherein the instructing by the nutrition delivery instruction module the nutrition supplier with instructions to deliver to the subject the supply of nutrition that corresponds to the recommended dietary regimen comprises providing to the nutrition supplier a description of foods that correspond to the recommended dietary regimen.

Embodiment D04. The computer program product of embodiment D03, wherein the instructing by the nutrition delivery instruction module the nutrition supplier with instructions to deliver to the subject the supply of nutrition that corresponds to the recommended dietary regimen comprises providing to the nutrition supplier a schedule for delivery to the subject of the foods that correspond to the recommended dietary regimen.

Embodiment D05. The computer program product of any one of embodiments D01-D04, wherein the computational nutrition system further comprises:
6) an update receipt module;
7) a revision module; and
8) a revision reporting module,
   wherein the method further comprises:
g) receiving by the update receipt module an update from the subject, wherein the update describes nutritional intake by the subject;
h) revising by the revision module the recommended dietary regimen for the subject based on the update from the subject to provide a revised dietary regimen; and
i) reporting to the subject by the revision reporting module the revised dietary regimen.

Embodiment D06. The computer program product of any one of embodiments D01-D05, wherein the computational nutrition system further comprises:
9) a health status module; and
10) a reporting module,
   wherein the method further comprises:
j) analyzing by the health status module the assay result to provide an analysis of a health status of the subject; and
k) reporting to the subject by the reporting module the analysis of the health status of the subject.

Embodiment D07. The computer program product of any one of embodiments D01-D06, wherein the health status characterizes an effect of gut microbiome activity on reproductive processes within about a year of giving birth.

Embodiment D08. The computer program product of any one of embodiments D01-D06, wherein the health status characterizes an effect of gut microbiome activity on pregnancy.

Embodiment D09. The computer program product of any one of embodiments D01-D06, wherein the health status characterizes an effect of gut microbiome activity on nursing.

Embodiment D10. The computer program product of any one of embodiments D01-D09, wherein the subject is pregnant.

Embodiment D11. The computer program product of any one of embodiments D01-D09, wherein the subject is preconception.

Embodiment D12. The computer program product of any one of embodiments D01-D09, wherein the subject is currently involved in a course of nursing a child of the birth.

Embodiment D13. The computer program product of any one of embodiments D01-D09, wherein the subject is lactating.

Embodiment D14. The computer program product of any one of embodiments D01-D13, wherein the subject is human.

Embodiment D15. The computer program product of any one of embodiments D01-D14, wherein the microbiome assay comprises next generation sequencing.

Embodiment D16. The computer program product of any one of embodiments D01-D15, wherein the microbiome assay comprises 16S rRNA sequencing.

Embodiment D17. The computer program product of any one of embodiments D01-D16, wherein the microbiome assay comprises 16S-ITS-23S rRNA sequencing.

Embodiment D18. The computer program product of any one of embodiments D01-D17, wherein the microbiome assay is an assay of a stool sample.

Embodiment D19. The computer program product of any one of embodiments D01-D17, wherein the microbiome assay is an assay of a saliva sample.

Embodiment D20. The computer program product of any one of embodiments D01-D17, wherein the microbiome assay is an assay of a vaginal fluid sample.

Embodiment D21. The computer program product of any one of embodiments D01-D17, wherein the microbiome assay is an assay of a blood sample.

Embodiment E01. A kit comprising:
a) a sheet of material configured to be applied to a toilet seat such that when a user sits on the toilet seat, the sheet of material forms a receptacle underneath an anus of the user;
b) disposable gloves;
c) a sample vial with a cap that forms a seal to the sample vial, wherein the cap comprises an elongated utensil component, wherein when the cap is sealed to the sample vial, the elongated utensil component extends into the sample vial;
d) a specimen pouch suitable to contain the sample vial; and
e) a shipping box suitable to contain the specimen pouch.

Embodiment E02. The kit of embodiment E01, further comprising written instructions on use of the sheet of material, the disposable gloves, the sample vial, the specimen pouch, and the shipping box.

Embodiment E03. The kit of any one of embodiments E01-E02, further comprising a form that contains written instructions for performance of a microbiome assay on a biological sample, wherein the biological sample is contained in the sample vial.

Embodiment E04. The method of embodiment E03, wherein the microbiome assay comprises next generation sequencing.

Embodiment E05. The method of embodiment E03, wherein the microbiome assay comprises 16S rRNA sequencing.

Embodiment E06. The method of embodiment E03, wherein the microbiome assay comprises 16S-ITS-23S rRNA sequencing.

Embodiment E07. The kit of any one of embodiments E01-E06, wherein the shipping box contains the specimen pouch, and the specimen pouch contains the sample vial.

Embodiment E08. The kit of any one of embodiments E01-E07, further comprising an identification label.

Embodiment E09. The kit of any one of embodiments E01-E08, further comprising a shipping label.

Embodiment E10. The kit of any one of embodiments E01-E09, further comprising a preservation reagent.

Embodiment F01. A method comprising:
a) sending to a subject a kit, wherein the kit comprises:
1) a sheet of material configured to be applied to a toilet seat such that when a user sits on the toilet seat, the sheet of material forms a receptacle underneath an anus of the user;
2) disposable gloves;
3) a sample vial with a cap that forms a seal to the sample vial, wherein the cap comprises an elongated utensil component, wherein when the cap is sealed to the sample vial, the elongated utensil component extends into the sample vial;
4) a specimen pouch suitable to contain the sample vial; and
5) a shipping box suitable to contain the specimen pouch; and
b) receiving from the subject the shipping box with the sample vial packed within the specimen pouch, with the specimen pouch packed within the shipping box, wherein the sample vial contains a biological sample of the subject when packed within the shipping box,
   wherein the subject is a female within about a year of giving birth.

Embodiment F02. The method of embodiment F01, wherein the kit further comprises written instructions on use of the sheet of material, the disposable gloves, the sample vial, the specimen pouch, and the shipping box.

Embodiment F03. The method of any one of embodiments F01-F02, wherein the kit further comprises a form that contains written instructions for performance of a microbiome assay on the biological sample.

Embodiment F04. The method of embodiment F03, wherein the microbiome assay comprises next generation sequencing.

Embodiment F05. The method of embodiment F03, wherein the microbiome assay comprises 16S rRNA sequencing.

Embodiment F06. The method of embodiment F03, wherein the microbiome assay comprises 16S-ITS-23S rRNA sequencing.

Embodiment F07. The method of any one of embodiments F01-F06, wherein the biological sample is stool.

Embodiment F08. The method of any one of embodiments F01-F07, further comprising analyzing the biological sample to determine a health status of the subject.

Embodiment F09. The method of embodiment F08, wherein the health status of the subject characterizes an effect of gut microbiome activity on reproductive processes within about a year of giving birth.

Embodiment F10. The method of embodiment F08, wherein the health status of the subject characterizes an effect of gut microbiome activity on pregnancy.

Embodiment F11. The method of embodiment F08, wherein the health status of the subject characterizes an effect of gut microbiome activity on nursing.

Embodiment F12. The method of any one of embodiments F01-F11, wherein the health status of the subject is risk of dysbiosis.

Embodiment F13. The method of any one of embodiments F01-F12, wherein the health status of the subject is risk of dysbiosis of a gut microbiome of the subject.

Embodiment F14. The method of any one of embodiments F01-F13, wherein the health status of the subject is risk of gestational diabetes.

Embodiment F15. The method of any one of embodiments F01-F14, wherein the health status of the subject is risk of preterm birth.

Embodiment F16. The method of any one of embodiments F01-F15, wherein the kit further comprises an identification label.

Embodiment F17. The method of any one of embodiments F01-F16, wherein the kit further comprises a shipping label.

Embodiment F18. The method of any one of embodiments F01-F17, wherein the kit further comprises a preservation reagent.

Embodiment F19. The method of any one of embodiments F01-F18, further comprising determining a recommended dietary regimen for the subject based on an analysis of the biological sample of the subject.

Embodiment F20. The method of embodiment F19, further comprising sending to the subject a supply of nutrition that corresponds to the recommended dietary regimen.

Embodiment F21. The method of embodiment F20, further comprising receiving from the subject a report of nutritional intake of the subject, and providing to the subject a revised dietary regimen based on the report of nutritional intake in response to receiving the report of nutritional intake.

Embodiment F22. The method of any one of embodiments F01-F21, wherein the subject is pregnant.

Embodiment F23. The method of any one of embodiments F01-F21, wherein the subject is preconception.

Embodiment F24. The method of any one of embodiments F01-F21, wherein the subject is currently involved in a course of nursing a child of the birth.

Embodiment F25. The method of any one of embodiments F01-F21, wherein the subject is lactating.

Embodiment F26. The method of any one of embodiments F01-F25, wherein the subject is human.

Embodiment G01. A method comprising:
a) sending by a subject a biological sample of the subject to a health service provider; and
b) receiving by the subject from the health service provider a recommended dietary regimen for the subject based on an assay of the biological sample of the subject,
   wherein the subject is a female within about a year of giving birth.

Embodiment G02. The method of embodiment G01, further comprising receiving by the subject from the health service provider a supply of nutrition that corresponds to the recommended dietary regimen.

Embodiment G03. The method of embodiment G02, further comprising sending by the subject to the health service provider a report of nutritional intake of the subject, and receiving by the subject from the health service provider a revised dietary regimen in response to sending the report of nutritional intake.

Embodiment G04. The method of any one of embodiments G01-G03, wherein the assay is a sequencing assay.

Embodiment G05. The method of any one of embodiments G01-G04, wherein the assay is a nucleic acid sequencing assay.

Embodiment G06. The method of any one of embodiments G01-G05, wherein the assay is a microbiome sequencing assay.

Embodiment G07. The method of any one of embodiments G01-G06, wherein the assay comprises next generation sequencing.

Embodiment G08. The method of any one of embodiments G01-G07, wherein the assay comprises 16S rRNA sequencing.

Embodiment G09. The method of any one of embodiments G01-G08, wherein the assay comprises 16S-ITS-23S rRNA sequencing.

Embodiment G10. The method of any one of embodiments G01-G09, wherein the assay is a sequencing assay of a gut microbiome of the subject.

Embodiment G11. The method of any one of embodiments G01-G10, wherein the biological sample is stool.

Embodiment G12. The method of any one of embodiments G01-G10, wherein the biological sample is blood.

Embodiment G13. The method of any one of embodiments G01-G10, wherein the biological sample is saliva.

Embodiment G14. The method of any one of embodiments G01-G10, wherein the biological sample is vaginal fluid.

Embodiment G15. The method of any one of embodiments G01-G14, further comprising receiving by the subject a report of a health status of the subject from the health service provider, wherein the report of the health status of the subject is based on the assay of the biological sample.

Embodiment G16. The method of embodiment G15, wherein the health status of the subject characterizes an effect of gut microbiome activity on reproductive processes within about a year of giving birth.

Embodiment G17. The method of embodiment G15, wherein the health status of the subject is an effect of gut microbiome activity on pregnancy.

Embodiment G18. The method of embodiment G15, wherein the health status of the subject characterizes an effect of gut microbiome activity on nursing.

Embodiment G19. The method of any one of embodiments G01-G18, wherein the health status of the subject is risk of dysbiosis.

Embodiment G20. The method of any one of embodiments G01-G19, wherein the health status of the subject is risk of dysbiosis of a gut microbiome of the subject.

Embodiment G21. The method of any one of embodiments G01-G20, wherein the health status of the subject is risk of gestational diabetes.

Embodiment G22. The method of any one of embodiments G01-G20, wherein the health status of the subject is risk of preterm birth.

Embodiment H01. A system comprising:
a) a computer hardware that:
1) receives a result of a microbiome assay of a subject; and
2) determines based on the result of the microbiome assay a recommended dietary regimen for improving a health status of the subject, wherein the determining is based in part on the subject being a female within about a year of giving birth; and
b) a nutrition distribution component that sends to the subject a supply of nutrition that corresponds to the recommended dietary regimen.

Embodiment H02. The system of embodiment H01, further comprising:
c) a laboratory that performs the microbiome assay on a biological sample of the subject to provide the result of the microbiome assay to the computer hardware.

Embodiment H03. The system of embodiment H02, wherein the biological sample is stool.

Embodiment H04. The system of embodiment H02, wherein the biological sample is blood.

Embodiment H05. The system of embodiment H02, wherein the biological sample is saliva.

Embodiment H06. The system of embodiment H02, wherein the biological sample is vaginal fluid.

Embodiment H07. The system of any one of embodiments H02-H06, further comprising:
d) a shipping component that sends an outgoing package to the subject, wherein the outgoing package contains a tool suitable to obtain the biological sample of the subject; and
e) a receiving component that receives an incoming package from the subject, wherein the incoming package contains the biological sample of the subject.

Embodiment H08. The system of embodiment H07, wherein the tool is a sample vial.

Embodiment H09. The system of any one of embodiments H01-H08, wherein the computer hardware further:
3) determines based on the result of the microbiome assay the health status of the subject; and
4) provides to the subject a report of the health status of the subject.

Embodiment H10. The system of any one of embodiments H01-H09, wherein the health status of the subject characterizes an effect of gut microbiome activity on reproductive processes within about a year of giving birth.

Embodiment H11. The system of any one of embodiments H01-H09, wherein the health status of the subject characterizes an effect of gut microbiome activity on pregnancy.

Embodiment H12. The system of any one of embodiments H01-H09, wherein the health status of the subject characterizes an effect of gut microbiome activity on nursing.

Embodiment H13. The system of any one of embodiments H01-H12, wherein the health status of the subject is risk of dysbiosis.

Embodiment H14. The system of any one of embodiments H01-H13, wherein the health status of the subject is risk of dysbiosis of a gut microbiome of the subject.

Embodiment H15. The system of any one of embodiments H01-H14, wherein the health status of the subject is risk of gestational diabetes.

Embodiment H16. The system of any one of embodiments H01-H14, wherein the health status of the subject is risk of preterm birth.

Embodiment H17. The system of any one of embodiments H01-H16, wherein the subject is pregnant.

Embodiment H18. The system of any one of embodiments H01-H16, wherein the subject is preconception.

Embodiment H19. The system of any one of embodiments H01-H16, wherein the subject is currently involved in a course of nursing a child of the birth.

Embodiment H20. The system of any one of embodiments H01-H16, wherein the subject is lactating.

Embodiment H21. The system of any one of embodiments H01-H20, wherein the subject is human.

Embodiment H22. The system of any one of embodiments H01-H21, wherein the microbiome assay is a sequencing assay.

Embodiment H23. The system of any one of embodiments H02-H22, wherein the microbiome assay is a nucleic acid sequencing assay.

Embodiment H24. The system of any one of embodiments H02-H23, wherein the microbiome assay comprises next generation sequencing.

Embodiment H25. The system of any one of embodiments H02-H24, wherein the microbiome assay comprises 16S rRNA sequencing.

Embodiment H26. The system of any one of embodiments H02-H25, wherein the microbiome assay comprises 16S-ITS-23S rRNA sequencing.

Embodiment I01. A computer program product encoded on a non-transitory computer-readable medium comprising code that instructs performance of a method, the method comprising:
a) providing a computational nutrition system, the computational nutrition system comprising:
1) a data receiving module;
2) a data processing module; and
3) a nutrition recommendation module;
b) receiving by the data receiving module data obtained from a microbiome assay of a subject, wherein the subject is a female within about a year of giving birth;
c) processing by the data processing module the data obtained from the microbiome assay to provide an assay result; and
d) determining by the nutrition recommendation module a recommended dietary regimen for the subject based on the assay result, wherein the determining is based in part on the subject being a female within about a year of giving birth.

Embodiment I02. The computer program product of embodiment I01, wherein the computational nutrition system further comprises:
4) a nutrition reporting module;
   wherein the method further comprises:
e) reporting to the subject by the nutrition reporting module the recommended dietary regimen for the subject.

Embodiment I03. The computer program product of embodiment I02, wherein the computational nutrition system further comprises:
4) a nutrition delivery instruction module,
   wherein the method further comprises:
e) instructing by the nutrition delivery instruction module a nutrition supplier with instructions to deliver to the subject a supply of nutrition that corresponds to the recommended dietary regimen.

Embodiment I04. The computer program product of embodiment I03, wherein the instructing by the nutrition delivery instruction module the nutrition supplier with instructions to deliver to the subject the supply of nutrition that corresponds to the recommended dietary regimen comprises providing to the nutrition supplier a description of foods that correspond to the recommended dietary regimen.

Embodiment I05. The computer program product of any one of embodiments I03-I04, wherein the instructing by the nutrition delivery instruction module the nutrition supplier with instructions to deliver to the subject the supply of nutrition that corresponds to the recommended dietary regimen comprises providing to the nutrition supplier a schedule for delivery to the subject of the foods that correspond to the recommended dietary regimen.

Embodiment I06. The computer program product of any one of embodiments I01-I05, wherein the computational nutrition system further comprises:
4) an update receipt module;
5) a revision module; and
6) a revision reporting module,
   wherein the method further comprises:
e) receiving by the update receipt module an update from the subject, wherein the update describes nutritional intake by the subject;
f) revising by the revision module the recommended dietary regimen for the subject based on the update from the subject to provide a revised dietary regimen; and
g) reporting to the subject by the revision reporting module the revised dietary regimen.

Embodiment I07. The computer program product of any one of embodiments I01-I06, wherein the subject is pregnant.

Embodiment I08. The computer program product of any one of embodiments I01-I06, wherein the subject is preconception.

Embodiment I09. The computer program product of any one of embodiments I01-I06, wherein the subject is currently involved in a course of nursing a child of the birth.

Embodiment I10. The computer program product of any one of embodiments I01-I06, wherein the subject is lactating.

Embodiment I11. The computer program product of any one of embodiments I01-I10, wherein the subject is human.

Embodiment I12. The computer program product of any one of embodiments I01-I11, wherein the microbiome assay is a sequencing assay.

Embodiment I13. The computer program product of any one of embodiments I01-I11, wherein the microbiome assay is a nucleic acid sequencing assay.

Embodiment I14. The computer program product of any one of embodiments I01-I11, wherein the microbiome assay comprises next generation sequencing.

Embodiment I15. The computer program product of any one of embodiments I01-I11, wherein the microbiome assay comprises 16S rRNA sequencing.

Embodiment I16. The computer program product of any one of embodiments I01-I11, wherein the microbiome assay comprises 16S-ITS-23S rRNA sequencing.

Embodiment I17. The computer program product of any one of embodiments I01-I16, wherein the microbiome assay is an assay on a biological sample that is stool.

Embodiment I18. The computer program product of any one of embodiments I01-I16, wherein the microbiome assay is an assay on a biological sample that is blood.

Embodiment I19. The computer program product of any one of embodiments I01-I16, wherein the microbiome assay is an assay on a biological sample that is saliva.

Embodiment I20. The computer program product of any one of embodiments I01-I16, wherein the microbiome assay is an assay on a biological sample that is vaginal fluid.

Embodiment J01. A method comprising:
a) receiving from a subject a report of nutritional intake of the subject; and
b) providing to the subject a revised dietary regimen based on the report,
   wherein the subject is a female within about a year of giving birth who is undergoing a dietary regimen based on an analysis of a microbiome assay of the subject.

Embodiment J02. The method of embodiment J01, wherein the subject is pregnant.

Embodiment J03. The method of embodiment J01, wherein the subject is preconception.

Embodiment J04. The method of embodiment J01, wherein the subject is currently involved in a course of nursing a child of the birth.

Embodiment J05. The method of embodiment J01, wherein the subject is lactating.

Embodiment J06. The method of any one of embodiments J01-J05, wherein the subject is human.

Embodiment J07. The method of any one of embodiments J01-J06, wherein the microbiome assay is a sequencing assay.

Embodiment J08. The method of any one of embodiments J01-J07, wherein the microbiome assay is a nucleic acid sequencing assay.

Embodiment J09. The method of any one of embodiments J01-J08, wherein the microbiome assay comprises next generation sequencing.

Embodiment J10. The method of any one of embodiments J01-J09, wherein the microbiome assay comprises 16S rRNA sequencing.

Embodiment J11. The method of any one of embodiments J01-J10, wherein the microbiome assay comprises 16S-ITS-23S rRNA sequencing.

Embodiment J12. The method of any one of embodiments J01-J11, wherein the microbiome assay is an assay on a biological sample that is stool.

Embodiment J13. The method of any one of embodiments J01-J11, wherein the microbiome assay is an assay on a biological sample that is blood.

Embodiment J14. The method of any one of embodiments J01-J11, wherein the microbiome assay is an assay on a biological sample that is saliva.

Embodiment J15. The method of any one of embodiments J01-J11, wherein the microbiome assay is an assay on a biological sample that is vaginal fluid.

Embodiment K01. A method comprising:
a) providing by a subject a report of nutritional intake of the subject to a health service provider; and
b) receiving by the subject a revised dietary regimen from the health service provider based on the report of nutritional intake,
   wherein the subject is a female within about a year of giving birth who is undergoing a dietary regimen based on an analysis of a microbiome assay of the subject.

Embodiment K02. The method of embodiment K01, wherein the subject is pregnant.

Embodiment K03. The method of embodiment K01, wherein the subject is preconception.

Embodiment K04. The method of embodiment K01, wherein the subject is currently involved in a course of nursing a child of the birth.

Embodiment K05. The method of embodiment K01, wherein the subject is lactating.

Embodiment K06. The method of any one of embodiments K01-K05, wherein the subject is human.

Embodiment K07. The method of any one of embodiments K01-K06, wherein the microbiome assay is a sequencing assay.

Embodiment K08. The method of any one of embodiments K01-K07, wherein the microbiome assay is a nucleic acid sequencing assay.

Embodiment K09. The method of any one of embodiments K01-K08, wherein the microbiome assay comprises next generation sequencing.

Embodiment K10. The method of any one of embodiments K01-K09, wherein the microbiome assay comprises 16S rRNA sequencing.

Embodiment K11. The method of any one of embodiments K01-K10, wherein the microbiome assay comprises 16S-ITS-23S rRNA sequencing.

Embodiment K12. The method of any one of embodiments K01-K11, wherein the microbiome assay is an assay on a biological sample that is stool.

Embodiment K13. The method of any one of embodiments K01-K11, wherein the microbiome assay is an assay on a biological sample that is blood.

Embodiment K14. The method of any one of embodiments K01-K11, wherein the microbiome assay is an assay on a biological sample that is saliva.

Embodiment K15. The method of any one of embodiments K01-K11, wherein the microbiome assay is an assay on a biological sample that is vaginal fluid.

Embodiment L01. A computer program product encoded on a non-transitory computer-readable medium comprising code that instructs performance of a method, the method comprising:
a) providing a computational nutrition system, the computational nutrition system comprising:
1) an update receipt module;
2) a revision module; and
3) a revision reporting module;
b) receiving by the update receipt module an update from a subject, wherein the update describes nutritional intake by the subject;
c) revising by the revision module the recommended dietary regimen for the subject based on the update from the subject to provide a revised dietary regimen, wherein the revised dietary regimen accounts for an effect of microbiome activity on a health status of the subject, wherein the health status characterizes an effect of gut microbiome activity on reproductive processes within about a year of giving birth; and
d) reporting to the subject by the revision reporting module the revised dietary regimen.

Embodiment L02. The computer program product of embodiment L01, wherein the health status of the subject characterizes an effect of gut microbiome activity on pregnancy.

Embodiment L03. The computer program product of embodiment L01, wherein the health status of the subject characterizes an effect of gut microbiome activity on nursing.

Embodiment L04. The computer program product of embodiment L01, wherein the health status of the subject is risk of dysbiosis.

Embodiment L05. The computer program product of any one of embodiments L01-L04, wherein the health status of the subject is risk of dysbiosis of a gut microbiome.

Embodiment L06. The computer program product of any one of embodiments L01-L05, wherein the health status of the subject is risk of gestational diabetes.

Embodiment L07. The computer program product of any one of embodiments L01-L05, wherein the health status of the subject is risk of preterm birth.

Embodiment L08. The computer program product of any one of embodiments L01-L07, wherein the subject is pregnant.

Embodiment L09. The computer program product of any one of embodiments L01-L07, wherein the subject is preconception.

Embodiment L10. The computer program product of any one of embodiments L01-L07, wherein the subject is currently involved in a course of nursing a child of the birth.

Embodiment L11. The computer program product of any one of embodiments L01-L07, wherein the subject is lactating.

Embodiment L12. The computer program product of any one of embodiments L01-L11, wherein the subject is human.

Embodiment M01. A method for identifying a likelihood of a pregnancy-related condition in a subject, the method comprising:
a) assaying microbial nucleic acids from a biological sample of the subject, thereby generating a microbiome profile of the subject; and
b) identifying the likelihood of the pregnancy-related condition in the subject based on the microbiome profile of the subject.

Embodiment M02. The method of embodiment M01, wherein the microbiome profile comprises microbial taxa and a relative abundance of microbial taxa.

Embodiment M03. The method of any one of embodiments M01-M02, wherein the likelihood of the pregnancy-related condition in the subject is based on an enrichment of a bacterial species in the biological sample.

Embodiment M04. The method of any one of embodiments M01-M03, wherein the likelihood of the pregnancy-related condition in the subject is based on a deficiency of a bacterial species in the biological sample.

Embodiment M05. The method of any one of embodiments M01-M04, further comprising determining a nutritional therapy that improves the pregnancy-related condition in the subject based on the microbiome profile of the subject.

Embodiment M06. The method of any one of embodiments M01-M05, further comprising determining a nutritional therapy that promotes a eubiotic state in the subject based on the microbiome profile of the subject.

Embodiment M07. The method of any one of embodiments M01-M06, wherein the assaying comprises genomic sequencing.

Embodiment M08. The method of any one of embodiments M01-M07, wherein the assaying comprises next generation sequencing.

Embodiment M09. The method of any one of embodiments M01-M08, wherein the assaying comprises 16S rRNA sequencing.

Embodiment M10. The method of any one of embodiments M01-M09, wherein the pregnancy-related condition is a dysbiotic state.

Embodiment M11. The method of any one of embodiments M01-M09, wherein the pregnancy-related condition is gestational diabetes.

Embodiment M12. The method of any one of embodiments M01-M09, wherein the pregnancy-related condition is preeclampsia.

Embodiment M13. The method of any one of embodiments M01-M12, wherein the subject is human.

Embodiment M14. The method of any one of embodiments M01-M13, wherein the subject is of childbearing age.

Embodiment M15. The method of any one of embodiments M01-M14, wherein the subject is pregnant.

Embodiment M16. The method of any one of embodiments M01-M15, wherein the subject is in a first trimester of pregnancy.

Embodiment M17. The method of any one of embodiments M01-M15, wherein the subject is in a second trimester of pregnancy.

Embodiment M18. The method of any one of embodiments M01-M15, wherein the subject is in a third trimester of pregnancy.

Embodiment M19. The method of any one of embodiments M01-M14, wherein the subject is not pregnant.

Embodiment M20. The method of any one of embodiments M01-M14, wherein the subject is trying to conceive.

Embodiment M21. The method of any one of embodiments M01-M14, wherein the subject is lactating.

Embodiment M22. The method of any one of embodiments M01-M13, wherein the subject is an infant.

Embodiment M23. The method of any one of embodiments M01-M22, wherein the biological sample comprises a fecal sample.

Embodiment M24. The method of any one of embodiments M01-M22, wherein the biological sample comprises an oral sample.

Embodiment M25. The method of any one of embodiments M01-M22, wherein the biological sample comprises a vaginal sample.

Embodiment N01. A method for identifying a likelihood of a pregnancy-related condition in a subject, the method comprising:
a) assaying microbial nucleic acids from a biological sample of the subject to detect a set of biomarkers; and
b) computer processing the set of biomarkers with a trained algorithm to identify the likelihood of the pregnancy-related condition in the subject.

Embodiment N02. The method of embodiment N01, wherein the trained algorithm is trained on microbiome sequencing data from subjects having the pregnancy-related condition and subjects not having the pregnancy-related condition.

Embodiment N03. The method of any one of embodiments N01-N02, wherein the trained algorithm is trained on microbiome sequencing data from pregnant subjects having the pregnancy-related condition and pregnant subjects not having the pregnancy-related condition.

Embodiment N04. The method of any one of embodiments N01-N03, wherein the trained algorithm is trained on nutritional habit data from subjects having the pregnancy-related condition and subjects not having the pregnancy-related condition.

Embodiment N05. The method of any one of embodiments N01-N04, wherein the trained algorithm is trained on nutritional habit data from pregnant subjects having the pregnancy-related condition and pregnant subjects not having the pregnancy-related condition.

Embodiment N06. The method of any one of embodiments N01-N05, wherein the trained algorithm is trained on blood glucose level data from subjects having the pregnancy-related condition and subjects not having the pregnancy-related condition.

Embodiment N07. The method of any one of embodiments N01-N06, wherein the trained algorithm is trained on blood glucose level data from pregnant subjects having the pregnancy-related condition and pregnant subjects not having the pregnancy-related condition.

Embodiment N08. The method of any one of embodiments N01-N07, wherein the assaying comprises genomic sequencing.

Embodiment N09. The method of any one of embodiments N01-N08, wherein the assaying comprises next generation sequencing.

Embodiment N10. The method of any one of embodiments N01-N09, wherein the assaying comprises 16S rRNA sequencing.

Embodiment N11. The method of any one of embodiments N01-N10, wherein the pregnancy-related condition is a dysbiotic state.

Embodiment N12. The method of any one of embodiments N01-N11, wherein the pregnancy-related condition is gestational diabetes.

Embodiment N13. The method of any one of embodiments N01-N11, wherein the pregnancy-related condition is preeclampsia.

Embodiment N14. The method of any one of embodiments N01-N13, wherein the subject is human.

Embodiment N15. The method of any one of embodiments N01-N14, wherein the subject is of childbearing age.

Embodiment N16. The method of any one of embodiments N01-N15, wherein the subject is pregnant.

Embodiment N17. The method of any one of embodiments N01-N16, wherein the subject is in a first trimester of pregnancy.

Embodiment N18. The method of any one of embodiments N01-N16, wherein the subject is in a second trimester of pregnancy.

Embodiment N19. The method of any one of embodiments N01-N16, wherein the subject is in a third trimester of pregnancy.

Embodiment N20. The method of any one of embodiments N01-N15, wherein the subject is not pregnant.

Embodiment N21. The method of any one of embodiments N01-N15, wherein the subject is trying to conceive.

Embodiment N22. The method of any one of embodiments N01-N15, wherein the subject is lactating.

Embodiment N23. The method of any one of embodiments N01-N14, wherein the subject is an infant.

Embodiment N24. The method of any one of embodiments N01-N23, wherein the biological sample comprises a fecal sample.

Embodiment N25. The method of any one of embodiments N01-N23, wherein the biological sample comprises an oral sample.

Embodiment N26. The method of any one of embodiments N01-N23, wherein the biological sample comprises a vaginal sample.

Embodiment O01. A system for detecting a pregnancy-related condition in a subject, the system comprising:
a) a computer-readable medium comprising a machine learning model classifier operable to classify a likelihood of the pregnancy-related condition in the subject based on a microbiome profile of the subject; and
b) a processor for executing instructions stored on the computer-readable medium.

Embodiment O02. The system of embodiment O01, wherein the microbiome profile comprises microbial taxa and a relative abundance of microbial taxa.

Embodiment O03. The system of any one of embodiments O01-O02, wherein the microbiome profile of the subject is generated from assaying microbial nucleic acids from a biological sample from the subject.

Embodiment O04. The system of embodiment O03, wherein the assaying comprises genomic sequencing.

Embodiment O05. The system of embodiment O03, wherein the assaying comprises next generation sequencing.

Embodiment O06. The system of embodiment O03, wherein the assaying comprises 16S rRNA sequencing.

Embodiment O07. The system of any one of embodiments O01-O06, wherein the biological sample comprises a fecal sample.

Embodiment O08. The system of any one of embodiments O01-O06, wherein the biological sample comprises an oral sample.

Embodiment O09. The system of any one of embodiments O01-O06, wherein the biological sample comprises a vaginal sample.

Embodiment O10. The system of any one of embodiments O01-O09, wherein the pregnancy-related condition is a dysbiotic state.

Embodiment O11. The system of any one of embodiments O01-O09, wherein the pregnancy-related condition is gestational diabetes.

Embodiment O12. The system of any one of embodiments O01-O09, wherein the pregnancy-related condition is preeclampsia.

Embodiment O13. The system of any one of embodiments O01-O12, wherein the subject is human.

Embodiment O14. The system of any one of embodiments O01-O13, wherein the subject is of childbearing age.

Embodiment O15. The system of any one of embodiments O01-O14, wherein the subject is pregnant.

Embodiment O16. The system of any one of embodiments O01-O15, wherein the subject is in a first trimester of pregnancy.

Embodiment O17. The system of any one of embodiments O01-O15, wherein the subject is in a second trimester of pregnancy.

Embodiment O18. The system of any one of embodiments O01-O15, wherein the subject is in a third trimester of pregnancy.

Embodiment O19. The system of any one of embodiments O01-O14, wherein the subject is not pregnant.

Embodiment O20. The system of any one of embodiments O01-O14, wherein the subject is trying to conceive.

Embodiment O21. The system of any one of embodiments O01-O14, wherein the subject is lactating.

Embodiment O22. The system of any one of embodiments O01-O13, wherein the subject is an infant.

Embodiment P01. A method of treating a dysbiosis in a subject in need thereof, the method comprising:
a) obtaining a microbiome composition of the subject;
b) determining a personalized nutritional therapy to the subject based on the microbiome composition of the subject; and
c) supplying to the subject a supply of nutrition based on the personalized nutritional therapy,
   wherein the personalized nutritional therapy treats the dysbiosis in the subject.

Embodiment P02. The method of embodiment P01, further comprising detecting in a biological sample of the subject the microbiome composition of the subject.

Embodiment P03. The method of embodiment P02, wherein the detecting comprises genomic sequencing of the biological sample.

Embodiment P04. The method of any one of embodiments P02-P03, wherein the detecting comprises next generation sequencing of the biological sample.

Embodiment P05. The method of any one of embodiments P02-P04, wherein the detecting comprises 16S rRNA sequencing of the biological sample.

Embodiment P06. The method of any one of embodiments P02-P05, wherein the biological sample comprises a fecal sample.

Embodiment P07. The method of any one of embodiments P02-P05, wherein the biological sample comprises an oral sample.

Embodiment P08. The method of any one of embodiments P02-P05, wherein the biological sample comprises a vaginal sample.

Embodiment P09. The method of any one of embodiments P01-P08, wherein the subject has gestational diabetes.

Embodiment P10. The method of any one of embodiments P01-P08, wherein the subject has preeclampsia.

Embodiment P11. The method of any one of embodiments P01-P10, wherein the subject is human.

Embodiment P12. The method of any one of embodiments P01-P11, wherein the subject is of childbearing age.

Embodiment P13. The method of any one of embodiments P01-P12, wherein the subject is pregnant.

Embodiment P14. The method of any one of embodiments P01-P13, wherein the subject is in a first trimester of pregnancy.

Embodiment P15. The method of any one of embodiments P01-P13, wherein the subject is in a second trimester of pregnancy.

Embodiment P16. The method of any one of embodiments P01-P13, wherein the subject is in a third trimester of pregnancy.

Embodiment P17. The method of any one of embodiments P01-P12, wherein the subject is not pregnant.

Embodiment P18. The method of any one of embodiments P01-P12, wherein the subject is trying to conceive.

Embodiment P19. The method of any one of embodiments P01-P12, wherein the subject is lactating.

Embodiment P20. The method of any one of embodiments P01-P11, wherein the subject is an infant.

Embodiment Q01. A method of treating or reducing a likelihood of a pregnancy-related condition in a subject in need thereof, the method comprising:
a) receiving by a subject from a telecommunications device a personalized nutritional therapy, wherein the personalized nutritional therapy is based on a microbiome profile of the subject; and
b) adhering by the subject to the personalized nutritional therapy over a time period,
   wherein the pregnancy-related condition is improved after the subject adheres to the personalized nutritional therapy over the time period, wherein the time period is at least a week.

Embodiment Q02. The method of embodiment Q01, wherein the microbiome profile comprises microbial taxa and a relative abundance of microbial taxa.

Embodiment Q03. The method of any one of embodiments Q01-Q02, wherein the microbiome profile of the subject is generated from assaying microbial nucleic acids from a biological sample from the subject.

Embodiment Q04. The method of embodiment Q03, wherein the assaying comprises genomic sequencing.

Embodiment Q05. The method of embodiment Q03, wherein the assaying comprises next generation sequencing.

Embodiment Q06. The method of embodiment Q03, wherein the assaying comprises 16S rRNA sequencing.

Embodiment Q07. The method of any one of embodiments Q01-Q06, wherein the biological sample comprises a fecal sample.

Embodiment Q08. The method of any one of embodiments Q01-Q06, wherein the biological sample comprises an oral sample.

Embodiment Q09. The method of any one of embodiments Q01-Q06, wherein the biological sample comprises a vaginal sample.

Embodiment Q10. The method of any one of embodiments Q01-Q09, wherein the pregnancy-related condition is a dysbiotic state.

Embodiment Q11. The method of any one of embodiments Q01-Q09, wherein the pregnancy-related condition is gestational diabetes.

Embodiment Q12. The method of any one of embodiments Q01-Q09, wherein the pregnancy-related condition is preeclampsia.

Embodiment Q13. The method of any one of embodiments Q01-Q12, wherein the subject is human.

Embodiment Q14. The method of any one of embodiments Q01-Q13, wherein the subject is of childbearing age.

Embodiment Q15. The method of any one of embodiments Q01-Q14, wherein the subject is pregnant.

Embodiment Q16. The method of any one of embodiments Q01-Q15, wherein the subject is in a first trimester of pregnancy.

Embodiment Q17. The method of any one of embodiments Q01-Q15, wherein the subject is in a second trimester of pregnancy.

Embodiment Q18. The method of any one of embodiments Q01-Q15, wherein the subject is in a third trimester of pregnancy.

Embodiment Q19. The method of any one of embodiments Q01-Q14, wherein the subject is not pregnant.

Embodiment Q20. The method of any one of embodiments Q01-Q14, wherein the subject is trying to conceive.

Embodiment Q21. The method of any one of embodiments Q01-Q14, wherein the subject is lactating.

Embodiment Q22. The method of any one of embodiments Q01-Q13, wherein the subject is an infant.

Embodiment Q23. The method of any one of embodiments Q01-Q22, wherein the time period is at least 25 days.

Embodiment Q24. The method of any one of embodiments Q01-Q22, wherein the time period is at least 3 months.

Embodiment Q25. The method of any one of embodiments Q01-Q22, wherein the time period is at least 6 months.

Embodiment Q26. The method of any one of embodiments Q01-Q22, wherein the time period is at least 1 year.

1. A method comprising analyzing a result of a gut microbiome sequencing assay of a biological sample of a subject to provide an outcome, and determining a health status of the subject based on the outcome, wherein the subject is a female within about a year of giving birth.

2. The method of claim 1, wherein the health status of the subject characterizes an effect of gut microbiome activity on reproductive processes within about a year of giving birth.

3. The method of claim 1, wherein the health status of the subject characterizes an effect of gut microbiome activity on pregnancy.

4. The method of claim 1, wherein the health status of the subject characterizes an effect of gut microbiome activity on nursing.

5. The method of claim 1, wherein the health status of the subject is risk of dysbiosis.

6. The method of claim 1, wherein the health status of the subject is risk of dysbiosis of a gut microbiome of the subject.

7. The method of claim 1, wherein the health status of the subject is risk of gestational diabetes.

8. The method of claim 1, wherein the health status of the subject is risk of preterm birth.

9. The method of claim 1, wherein the biological sample is stool.

10. The method of claim 1, further comprising, prior to the analyzing, performing the gut microbiome sequencing assay on the biological sample of the subject.

11. The method of claim 1, wherein the performing the gut microbiome sequencing assay comprises next generation sequencing.

12. The method of claim 1, wherein the performing the gut microbiome sequencing assay comprises 16S rRNA sequencing.

13. The method of claim 1, wherein the performing the gut microbiome sequencing assay comprises 16S-ITS-23S rRNA sequencing.

14. The method of claim 1, further comprising determining a recommended dietary regimen for the subject based on the result of the gut microbiome sequencing assay of the biological sample of the subject.

15. The method of claim 1, further comprising sending to the subject a supply of nutrition that corresponds to the recommended dietary regimen.

16. The method of claim 1, further comprising receiving from the subject a report of nutritional intake of the subject, and providing to the subject a revised dietary regimen in response to receiving the report of nutritional intake.

17. The method of claim 1, wherein the subject is pregnant.

18. The method of claim 1, wherein the subject is preconception.

19. The method of claim 1, wherein the subject is currently involved in a course of nursing a child of the birth.

20. The method of claim 1, wherein the subject is lactating.

21. The method of claim 1, wherein the subject is human.

22-344. (canceled)
